(19) United States
(12) Patent Application Publication
(10) Pub. No.: US 2026/0234155 A1
(43) Pub. Date: Aug. 13, 2026

(54) SOLID FORMS OF A WEE1 INHIBITOR

(71) Applicant: Zeno Management, Inc., San Diego, CA (US)

(72) Inventors: Matthew J. Peterson, San Diego, CA (US); Aditya Krishnan Unni, San Diego, CA (US); Xuejun Karl Liu, Arcadia, CA (US); Kaushalendra Chaturvedi, Somerset, NJ (US); Catrina Jou, Edison, NJ (US)

(21) Appl. No.: 19/638,544

(22) Filed: Apr. 3, 2026

Related U.S. Application Data
(63) Continuation of application No. PCT/US2024/049911, filed on Oct. 4, 2024.
(60) Provisional application No. 63/588,392, filed on Oct. 6, 2023.

(51) Int. Cl. C07D 487/04

(52) U.S. Cl. CPC C07D 487/04

(57) ABSTRACT

Solid forms of Compound A are described herein. The polymorphic forms described herein are WEE1 inhibitors. Processes and intermediates described herein are useful for making the Compound A and various polymorphic forms. Such forms are useful for treating diseases or conditions that are mediated by WEE1, including conditions characterized by excessive cellular proliferation, such as cancer.

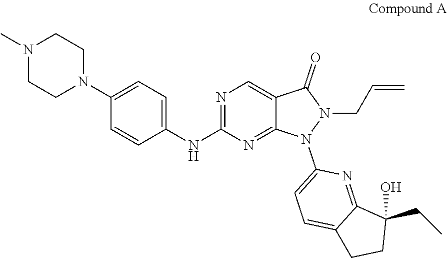 [SMILES: C=CCn1c(=O)c2cnc(Nc3ccc(N4CCN(C)CC4)cc3)nc2n1-c1ccc2c(n1)[C@@](O)(CC)CC2]

### INCORPORATION BY REFERENCE TO ANY PRIORITY APPLICATIONS

Any and all applications for which a foreign or domestic priority claim is identified, for example, in the Application Data Sheet or Request as filed with the present application, are hereby incorporated by reference under 37 CFR 1.57, and Rules 4.18 and 20.6, including U.S. Provisional Application No. 63/588,392, filed Oct. 6, 2023, which is incorporated by reference in its entirety. The present application is a continuation of PCT/US2024/049911, filed Oct. 4, 2024, which claims priority to U.S. Provisional Application No. 63/588,392, filed Oct. 6, 2023, each of which is incorporated by reference in their entireties, including any drawings.

### FIELD

The present application relates to processes and intermediates useful for making WEE1 inhibitor compounds, solid forms of such compounds and methods of using them to treat conditions characterized by excessive cellular proliferation, such as cancer.

### BACKGROUND

WEE1 kinase plays a role in the G2-M cell-cycle checkpoint arrest for DNA repair before mitotic entry. Normal cells repair damaged DNA during G1 arrest. Cancer cells often have a deficient G1-S checkpoint and depend on a functional G2-M checkpoint for DNA repair. WEE1 is overexpressed in various cancer types.

### SUMMARY

The present application relates to processes and intermediates useful for making solid forms of a WEE1 inhibitor compound and methods of using them to treat conditions characterized by excessive cellular proliferation, such as cancer.

Described herein are solid forms of (R)-2-allyl-1-(7-ethyl-7-hydroxy-6,7-dihydro-5H-cyclopenta[b]pyridin-2-yl)-6-((4-(4-methylpiperazin-1-yl)phenyl)amino)-1,2-dihydro-3H-pyrazolo[3,4-d]pyrimidin-3-one (Compound A, Azenosertib),

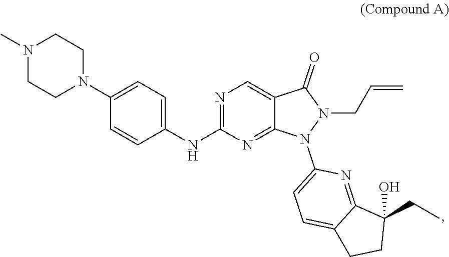 [SMILES: C=CCn1c(=O)c2cnc(Nc3ccc(N4CCN(C)CC4)cc3)nc2n1-c1ccc2c(n1)[C@@](O)(CC)CC2]

or any hydrates, solvates, and/or pharmaceutically acceptable salts thereof.

In some embodiments, a solid form of Compound A is selected from Form 1, Form 2, Form 3, Form 4, Form 5, Form 6, Form 7, Form 8, Form 9, Form 10, Form 11 and Form 12 of Compound A. In other embodiments, a solid form is characterized by X-Ray Powder Diffraction (XRPD), Differential Scanning calorimetry (DSC) and/or Thermo-Gravimetric Analysis (TGA), including as described herein.

In one aspect, a solid form of Compound A is Solid Form 1 of (R)-2-allyl-1-(7-ethyl-7-hydroxy-6,7-dihydro-5H-cyclopenta[b]pyridin-2-yl)-6-((4-(4-methylpiperazin-1-yl)phenyl)amino)-1,2-dihydro-3H-pyrazolo[3,4-d]pyrimidin-3-one,

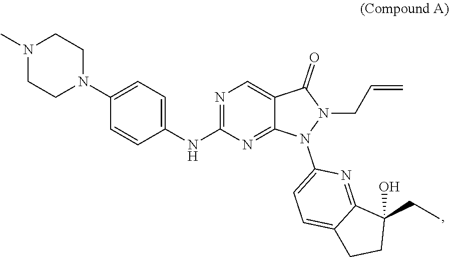 [SMILES: C=CCn1c(=O)c2cnc(Nc3ccc(N4CCN(C)CC4)cc3)nc2n1-c1ccc2c(n1)[C@@](O)(CC)CC2]

wherein said Solid Form 1 of Compound A has an X-ray powder diffraction pattern comprising one, two, three, four, or five peaks at about 4.91° 2θ±0.2° 2θ, about 5.98° 2θ±0.2° 2θ, about 9.77° 2θ±0.2° 2θ, about 10.64° 2θ±0.2° 2θ, or about 17.40° 2θ±0.2° 2θ.

In some embodiments, Solid Form 1 has an X-ray powder diffraction pattern substantially similar to the X-ray powder diffraction XRPD pattern of FIG. 1A.

In some embodiments, Solid Form 1 has a Differential Scanning calorimetry (DSC) thermogram substantially similar to the DSC thermogram of FIG. 1B.

In some embodiments, Solid Form 1 has a thermal gravimetric analysis (TGA) thermogram substantially similar to the TGA thermogram of FIG. 1B.

In some embodiments, Solid Form 1 has DSC endotherm peak at about 126° C., about 149° C. and/or about 168° C.

In some embodiments, Solid Form 1 has a first DSC endotherm peak at about 126° C., a second DSC endotherm peak at about 149° C., and a third DSC endotherm peak at about 168° C.

In some embodiments, Solid Form 1 has a weight loss of about 2.78 wt. % when heated from about room temperature to about 150° C.

In some embodiments, Solid Form 1 is substantially free of any other solid form of Compound A, such as those described herein and including Solid Forms 2-12 of Compound A.

In one aspect, a solid form of Compound A is Solid Form 2 of (R)-2-allyl-1-(7-ethyl-7-hydroxy-6,7-dihydro-5H-cyclopenta[b]pyridin-2-yl)-6-((4-(4-methylpiperazin-1-yl)phenyl)amino)-1,2-dihydro-3H-pyrazolo[3,4-d]pyrimidin-3-one,

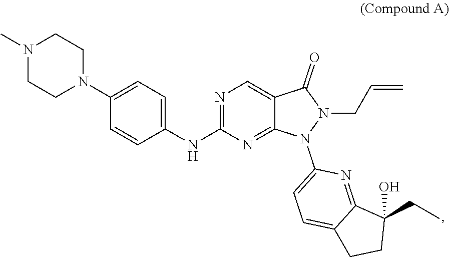 [SMILES: C=CCn1c(=O)c2cnc(Nc3ccc(N4CCN(C)CC4)cc3)nc2n1-c1ccc2c(n1)[C@@](O)(CC)CC2]

wherein said Solid Form 2 has an X-ray powder diffraction pattern comprising one, two, three, four, or five peaks at about 4.22° 2θ±0.2° 2θ, about 4.99° 2θ±0.2° 2θ, about 5.74° 2θ±0.2° 2θ, about 10.17° 2θ±0.2° 2θ, or about 16.04° 2θ±0.2° 2θ.

In some embodiments, Solid Form 2 has an X-ray powder diffraction pattern substantially similar to the X-ray powder diffraction pattern of FIG. 2A.

In some embodiments, Solid Form 2 has a Differential Scanning calorimetry (DSC) thermogram substantially similar to the DSC thermogram of FIG. 2B.

In some embodiments, Solid Form 2 has a thermal gravimetric analysis (TGA) thermogram substantially similar to the TGA thermogram of FIG. 2B.

In some embodiments, Solid Form 2 has DSC endotherm peak at about 134° C. and/or about 169° C., and/or has DSC exotherm peak at about 153° C.

In some embodiments, Solid Form 2 has a first DSC endotherm peak at about 134° C., a second DSC endotherm peak at about 169° C., and a first DSC exotherm peak at about 153° C.

In some embodiments, Solid Form 2 has a weight loss of about 1.20 wt. % when heated from about room temperature to about 150° C.

In some embodiments, Solid Form 2 is substantially free of any other solid form of Compound A, such as those described herein and including Solid Forms 1 and 3-12.

In one aspect, a solid form of Compound A is Solid Form 3 of (R)-2-allyl-1-(7-ethyl-7-hydroxy-6,7-dihydro-5H-cyclopenta[b]pyridin-2-yl)-6-((4-(4-methylpiperazin-1-yl)phenyl)amino)-1,2-dihydro-3H-pyrazolo[3,4-d]pyrimidin-3-one,

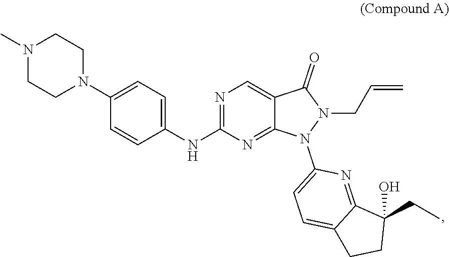 [SMILES: C=CCn1c(=O)c2cnc(Nc3ccc(N4CCN(C)CC4)cc3)nc2n1-c1ccc2c(n1)[C@@](O)(CC)CC2]

wherein said Solid Form 3 has an X-ray powder diffraction pattern comprising one, two, three, four, or five peaks at about 3.42° 2θ±0.2° 2θ, about 4.51° 2θ±0.2° 2θ, about 5.87° 2θ±0.2° 2θ, about 10.10° 2θ±0.2° 2θ, or about 16.67° 2θ±0.2° 2θ.

In some embodiments, Solid Form 3 has an X-ray powder diffraction pattern substantially similar to the X-ray powder diffraction pattern of FIG. 3A.

In some embodiments, Solid Form 3 has a Differential Scanning calorimetry (DSC) thermogram substantially similar to the DSC thermogram of FIG. 3B.

In some embodiments, Solid Form 3 has a thermal gravimetric analysis (TGA) thermogram substantially similar to the TGA thermogram of FIG. 3B.

In some embodiments, Solid Form 3 has DSC endotherm peak at about 119° C., about 140° C. and/or about 167° C., and/or has DSC exotherm peak at about 156° C.

In some embodiments, Solid Form 3 has a first DSC endotherm peak at about 119° C., a second DSC endotherm peak at about 140° C., a third DSC endotherm peak at about 167° C., and a first DSC exotherm peak at about 156° C.

In some embodiments, Solid Form 3 has a weight loss of about 2.26 wt. % when heated from about room temperature to about 150° C.

In some embodiments, Solid Form 3 is substantially free of any other solid form of Compound A, such as those described herein and including Solid Forms 1, 2 and 4-12.

In one aspect, a solid form of Compound A is Solid Form 4 of (R)-2-allyl-1-(7-ethyl-7-hydroxy-6,7-dihydro-5H-cyclopenta[b]pyridin-2-yl)-6-((4-(4-methylpiperazin-1-yl)phenyl)amino)-1,2-dihydro-3H-pyrazolo[3,4-d]pyrimidin-3-one,

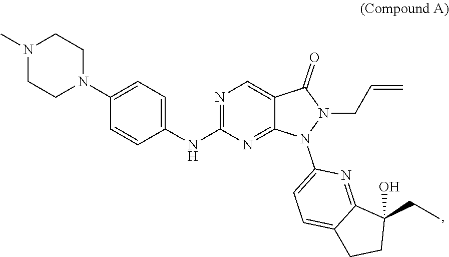 [SMILES: C=CCn1c(=O)c2cnc(Nc3ccc(N4CCN(C)CC4)cc3)nc2n1-c1ccc2c(n1)[C@@](O)(CC)CC2]

wherein said Solid Form 4 has an X-ray powder diffraction pattern comprising one, two, three, four, or five peaks at about 4.16° 2θ±0.2° 2θ, about 5.18° 2θ±0.2° 2θ, about 5.61° 2θ±0.2° 2θ, about 10.39° 2θ±0.2° 2θ, or about 16.65° 2θ±0.2° 2θ.

In some embodiments, Solid Form 4 has an X-ray powder diffraction pattern substantially similar to the X-ray powder diffraction pattern of FIG. 4.

In some embodiments, Solid Form 4 substantially free of any other solid form of Compound A, such as those described herein and including Solid Forms 1-3 and 5-12.

In one aspect, a solid form of Compound A is Solid Form 5 of (R)-2-allyl-1-(7-ethyl-7-hydroxy-6,7-dihydro-5H-cyclopenta[b]pyridin-2-yl)-6-((4-(4-methylpiperazin-1-yl)phenyl)amino)-1,2-dihydro-3H-pyrazolo[3,4-d]pyrimidin-3-one,

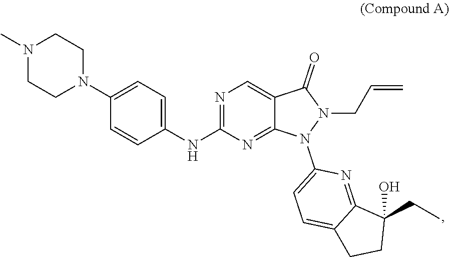 [SMILES: C=CCn1c(=O)c2cnc(Nc3ccc(N4CCN(C)CC4)cc3)nc2n1-c1ccc2c(n1)[C@@](O)(CC)CC2]

wherein said Solid Form 5 has an X-ray powder diffraction pattern comprising one, two, three, four, or five peaks at about 3.40° 2θ±0.2° 2θ, about 4.48° 2θ±0.2° 2θ, about 5.18° 2θ±0.2° 2θ, about 5.86° 2θ±0.2° 2θ, or about 10.30° 2θ±0.2° 2θ.

In some embodiments, Solid Form 5 has an X-ray powder diffraction pattern substantially similar to the X-ray powder diffraction pattern of FIG. 5A.

In some embodiments, Solid Form 5 has a Differential Scanning calorimetry (DSC) thermogram substantially similar to the DSC thermogram of FIG. 5B.

In some embodiments, Solid Form 5 has a thermal gravimetric analysis (TGA) thermogram substantially similar to the TGA thermogram of FIG. 5B.

In some embodiments, Solid Form 5 has a weight loss of about 9.08 wt. % when heated from about room temperature to about 150° C.

In some embodiments, Solid Form 5 is substantially free of any other solid form of Compound A, such as those described herein and including Solid Forms 1~4 and 6-12.

In one aspect, a solid form of Compound A is Solid Form 6 of (R)-2-allyl-1-(7-ethyl-7-hydroxy-6,7-dihydro-5H-cyclopenta[b]pyridin-2-yl)-6-((4-(4-methylpiperazin-1-yl)phenyl)amino)-1,2-dihydro-3H-pyrazolo[3,4-d]pyrimidin-3-one,

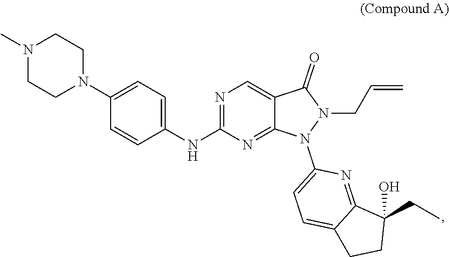 [SMILES: C=CCn1c(=O)c2cnc(Nc3ccc(N4CCN(C)CC4)cc3)nc2n1-c1ccc2c(n1)[C@@](O)(CC)CC2]

wherein said Solid Form 6 has an X-ray powder diffraction pattern comprising one, two, three, four, or five peaks at about 5.25° 2θ±0.2° 2θ, about 11.26° 2θ±0.2° 2θ, about 12.70° 2θ±0.2° 2θ, about 15.34° 2θ±0.2° 2θ, or about 16.71° 2θ±0.2° 2θ.

In some embodiments, Solid Form 6 has an X-ray powder diffraction pattern substantially similar to the X-ray powder diffraction pattern of FIG. 6A.

In some embodiments, Solid Form 6 has a Differential Scanning calorimetry (DSC) thermogram substantially similar to the DSC thermogram of FIG. 6B.

In some embodiments, Solid Form 6 has a thermal gravimetric analysis (TGA) thermogram substantially similar to the TGA thermogram of FIG. 6B.

In some embodiments, Solid Form 6 has DSC endotherm peak at about 142° C.

In some embodiments, Solid Form 6 has a weight loss of about 0.75 wt. % when heated from about room temperature to about 100° C.

In some embodiments, Solid Form 6 is substantially free of any other solid form of Compound A, such as those described herein and including Solid Forms 1-5 and 7-12.

In one aspect, a solid form of Compound A is Solid Form 7 of (R)-2-allyl-1-(7-ethyl-7-hydroxy-6,7-dihydro-5H-cyclopenta[b]pyridin-2-yl)-6-((4-(4-methylpiperazin-1-yl)phenyl)amino)-1,2-dihydro-3H-pyrazolo[3,4-d]pyrimidin-3-one,

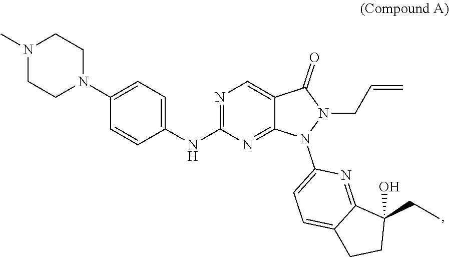 [SMILES: C=CCn1c(=O)c2cnc(Nc3ccc(N4CCN(C)CC4)cc3)nc2n1-c1ccc2c(n1)[C@@](O)(CC)CC2]

wherein said Solid Form 7 has an X-ray powder diffraction pattern comprising one, two, three, four, or five peaks at about 5.16° 2θ±0.2° 2θ, about 9.34° 2θ±0.2° 2θ, about 16.40° 2θ±0.2° 2θ, about 16.52° 2θ±0.2° 2θ, or about 22.44° 2θ±0.2° 2θ.

In some embodiments, Solid Form 7 has an X-ray powder diffraction pattern substantially similar to the X-ray powder diffraction pattern of FIG. 7A.

In some embodiments, Solid Form 7 has a Differential Scanning calorimetry (DSC) thermogram substantially similar to the DSC thermogram of FIG. 7B.

In some embodiments, Solid Form 7 has a thermal gravimetric analysis (TGA) thermogram substantially similar to the TGA thermogram of FIG. 7B.

In some embodiments, Solid Form 7 has DSC endotherm peak at about 80° C. and/or 169° C., and/or has DSC exotherm peak at about 143° C.

In some embodiments, Solid Form 7 has a first DSC endotherm peak at about 80° C., a second DSC endotherm peak at about 169° C., and a first DSC exotherm peak at about 143° C.

In some embodiments, Solid Form 7 has a weight loss of about 18.1 wt. % when heated from about room temperature to about 150° C.

In some embodiments, Solid Form 7 is substantially free of any other solid form of Compound A, such as those described herein and including Solid Forms 1-6 and 8-12.

In one aspect, a solid form of Compound A is Solid Form 8 of (R)-2-allyl-1-(7-ethyl-7-hydroxy-6,7-dihydro-5H-cyclopenta[b]pyridin-2-yl)-6-((4-(4-methylpiperazin-1-yl)phenyl)amino)-1,2-dihydro-3H-pyrazolo[3,4-d]pyrimidin-3-one,

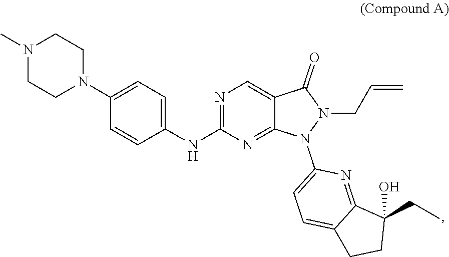 [SMILES: C=CCn1c(=O)c2cnc(Nc3ccc(N4CCN(C)CC4)cc3)nc2n1-c1ccc2c(n1)[C@@](O)(CC)CC2]

wherein said Solid Form 8 has an X-ray powder diffraction pattern comprising one, two, three, four, or five peaks at about 5.02° 2θ±0.2° 2θ, about 11.81° 2θ±0.2° 2θ, about 15.03° 2θ±0.2° 2θ, about 16.41° 2θ±0.2° 2θ, or about 20.09° 2θ±0.2° 2θ.

In some embodiments, Solid Form 8 has an X-ray powder diffraction pattern substantially similar to the X-ray powder diffraction pattern of FIG. 8A.

In some embodiments, Solid Form 8 has a Differential Scanning calorimetry (DSC) thermogram substantially similar to the DSC thermogram of FIG. 8B.

In some embodiments, Solid Form 8 has a thermal gravimetric analysis (TGA) thermogram substantially similar to the TGA thermogram of FIG. 8B.

In some embodiments, Solid Form 8 has DSC endotherm peak at about 75° C. and/or about 99° C.

In some embodiments, Solid Form 8 has a first DSC endotherm peak at about 75° C., and a second DSC endotherm peak at about 99° C.

In some embodiments, Solid Form 8 has a weight loss of about 6.0 wt. % when heated from about room temperature to about 125° C., and/or about 9.55 wt. % when heated from about 125° C. to about 252° C.

In some embodiments, Solid Form 8 is substantially free of any other solid form of Compound A, such as those described herein and including Solid Forms 1-7 and 9-12.

In one aspect, a solid form of Compound A is Solid Form 9 of (R)-2-allyl-1-(7-ethyl-7-hydroxy-6,7-dihydro-5H-cyclopenta[b]pyridin-2-yl)-6-((4-(4-methylpiperazin-1-yl)phenyl)amino)-1,2-dihydro-3H-pyrazolo[3,4-d]pyrimidin-3-one,

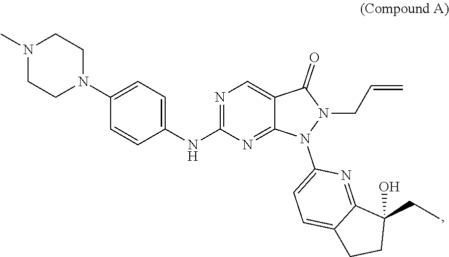 [SMILES: C=CCn1c(=O)c2cnc(Nc3ccc(N4CCN(C)CC4)cc3)nc2n1-c1ccc2c(n1)[C@@](O)(CC)CC2]

wherein said Solid Form 9 has an X-ray powder diffraction pattern comprising one, two, three, four, or five peaks at about 4.20° 2θ±0.2° 2θ, about 4.47° 2θ±0.2° 2θ, about 5.20° 2θ±0.2° 2θ, about 6.43° 2θ±0.2° 2θ, or about 10.46° 2θ±0.2° 2θ.

In some embodiments, Solid Form 9 has an X-ray powder diffraction pattern substantially similar to the X-ray powder diffraction pattern of FIG. 9A.

In some embodiments, Solid Form 9 has a Differential Scanning calorimetry (DSC) thermogram substantially similar to the DSC thermogram of FIG. 9B.

In some embodiments, Solid Form 9 has a thermal gravimetric analysis (TGA) thermogram substantially similar to the TGA thermogram of FIG. 9B.

In some embodiments, Solid Form 9 has DSC endotherm peak at about 130° C., and/or about 168° C., and/or has DSC exotherm peak at about 143° C.

In some embodiments, Solid Form 9 has a first DSC endotherm peak at about 130° C., the second DSC endotherm peak at about 168° C., and the first DSC exotherm peak at about 143° C.

In some embodiments, Solid Form 9 has a weight loss of about 0.99 wt. % when heated from about room temperature to about 150° C.

In some embodiments, Solid Form 9 is substantially free of any other solid form of Compound A, such as those described herein and including Solid Forms 1-8 and 10-12.

In one aspect, a solid form of Compound A is Solid Form 10 of (R)-2-allyl-1-(7-ethyl-7-hydroxy-6,7-dihydro-5H-cyclopenta[b]pyridin-2-yl)-6-((4-(4-methylpiperazin-1-yl)phenyl)amino)-1,2-dihydro-3H-pyrazolo[3,4-d]pyrimidin-3-one,

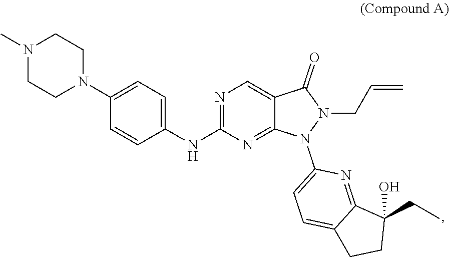 [SMILES: C=CCn1c(=O)c2cnc(Nc3ccc(N4CCN(C)CC4)cc3)nc2n1-c1ccc2c(n1)[C@@](O)(CC)CC2]

wherein said Solid Form 10 has an X-ray powder diffraction pattern comprising one, two, three, four, or five peaks at about 4.96° 2θ±0.2° 2θ, about 5.13° 2θ±0.2° 2θ, about 5.53° 2θ±0.2° 2θ, about 10.22° 2θ±0.2° 2θ, or about 11.15° 2θ±0.2° 2θ.

In some embodiments, Solid Form 10 has an X-ray powder diffraction pattern substantially similar to the X-ray powder diffraction pattern of FIG. 10.

In some embodiments, Solid Form 10 is substantially free of any other solid form of Compound A, such as those described herein and including Solid Forms 1-9, 11 and 12.

In one aspect, a solid form of Compound A is Solid Form 11 of (R)-2-allyl-1-(7-ethyl-7-hydroxy-6,7-dihydro-5H-cyclopenta[b]pyridin-2-yl)-6-((4-(4-methylpiperazin-1-yl)phenyl)amino)-1,2-dihydro-3H-pyrazolo[3,4-d]pyrimidin-3-one,

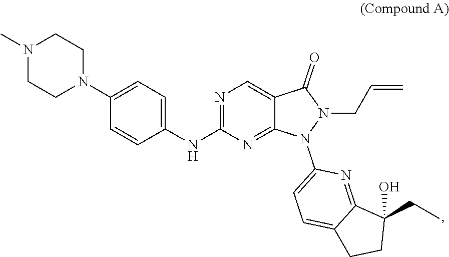 [SMILES: C=CCn1c(=O)c2cnc(Nc3ccc(N4CCN(C)CC4)cc3)nc2n1-c1ccc2c(n1)[C@@](O)(CC)CC2]

wherein said Solid Form 11 has an X-ray powder diffraction pattern comprising one, two, three, four, or five peaks at about 4.38° 2θ±0.2° 2θ, about 5.20° 2θ±0.2° 2θ, about 6.43° 2θ±0.2° 2θ, about 10.45° 2θ±0.2° 2θ, or about 12.61° 2θ±0.2° 2θ.

In some embodiments, Solid Form 11 has an X-ray powder diffraction pattern substantially similar to the X-ray powder diffraction pattern of FIG. 11A.

In some embodiments, Solid Form 11 has a Differential Scanning calorimetry (DSC) thermogram substantially similar to the DSC thermogram of FIG. 11B.

In some embodiments, Solid Form 11 has a thermal gravimetric analysis (TGA) thermogram substantially similar to the TGA thermogram of FIG. 11B.

In some embodiments, Solid Form 11 has DSC endotherm peak at about 134° C., and/or about 168° C.

In some embodiments, Solid Form 11 has a first DSC endotherm peak at about 134° C., and a second DSC endotherm peak at about 168° C.

In some embodiments, Solid Form 11 has a weight loss of about 1.13 wt. % when heated from about room temperature to about 100° C.

In some embodiments, Solid Form 11 is substantially free of any other solid form of Compound A, such as those described herein and including Solid Forms 1-10 and 12.

In one aspect, a solid form of Compound A is Solid Form 12 of (R)-2-allyl-1-(7-ethyl-7-hydroxy-6,7-dihydro-5H-cyclopenta[b]pyridin-2-yl)-6-((4-(4-methylpiperazin-1-yl)phenyl)amino)-1,2-dihydro-3H-pyrazolo[3,4-d]pyrimidin-3-one,

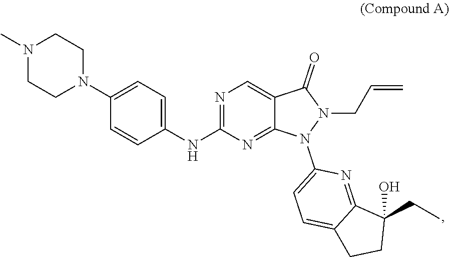 [SMILES: C=CCn1c(=O)c2cnc(Nc3ccc(N4CCN(C)CC4)cc3)nc2n1-c1ccc2c(n1)[C@@](O)(CC)CC2]

wherein said Solid Form 12 has an X-ray powder diffraction pattern comprising one, two, three, four, or five peaks at about 5.23° 2θ±0.2° 2θ, about 15.64° 2θ±0.2° 2θ, about 16.48° 2θ±0.2° 2θ, about 16.67° 2θ±0.2° 2θ, or about 21.34° 2θ±0.2° 2θ.

In some embodiments, Solid Form 12 has an X-ray powder diffraction pattern substantially similar to the X-ray powder diffraction pattern of FIG. 12A.

In some embodiments, Solid Form 12 has a Differential Scanning calorimetry (DSC) thermogram substantially similar to the DSC thermogram of FIG. 12B.

In some embodiments, Solid Form 12 has a thermal gravimetric analysis (TGA) thermogram substantially similar to the TGA thermogram of FIG. 12B.

In some embodiments, Solid Form 12 has DSC endotherm peak at about 90° C.

In some embodiments, Solid Form 12 has a weight loss of about 8.66 wt. % when heated from about room temperature to about 150° C.

In some embodiments, Solid Form 12 is substantially free of any other solid form of Compound A, such as those described herein and including Solid Forms 1-11.

In one aspect, provided herein is a pharmaceutical composition comprising an effective amount of a solid form of Compound A described herein (e.g., any one of Solid Forms 1-12), and a pharmaceutically acceptable carrier, diluent, excipient, or combination thereof.

In one aspect, provided herein is a method for ameliorating or treating a malignant growth or tumor in a patient in need thereof, said method comprising administering to the patient an effective amount of a solid form of Compound A described herein (e.g., any one of Solid Forms 1-12), or any pharmaceutical composition thereof.

In one aspect, provided herein is a method for ameliorating or treating a malignant growth or tumor in a patient in need thereof, said method comprising contacting the malignant grown or tumor with an effective amount of a solid form of Compound A described herein (e.g., any one of Solid Forms 1-12), or any pharmaceutical composition thereof.

In some embodiments, a malignant growth or tumor is due to a cancer, wherein said cancer is selected from a breast cancer, a cervical cancer, an ovarian cancer, a uterine cancer, a vaginal cancer, a vulvar cancer, a brain cancer, a cervicocerebral cancer, an esophageal cancer, a thyroid cancer, a small cell cancer, a non-small cell cancer, a lung cancer, a stomach cancer, a gallbladder/bile duct cancer, a liver cancer, a pancreatic cancer, a colon cancer, a rectal cancer, a choriocarcinoma, an uterus body cancer, an uterocervical cancer, a renal pelvis/ureter cancer, a bladder cancer, a prostate cancer, a penis cancer, a testicular cancer, a fetal cancer, a Wilms' cancer, a skin cancer, a malignant melanoma, a neuroblastoma, an osteosarcoma, an Ewing's tumor, a soft part sarcoma, an acute leukemia, a chronic lymphatic leukemia, a chronic myelocytic leukemia, polycythemia vera, a malignant lymphoma, multiple myeloma, a Hodgkin's lymphoma and a non-Hodgkin's lymphoma.

In some embodiments, a cancer is a breast cancer, a cervical cancer, an ovarian cancer, a uterine cancer, a vaginal cancer, or a vulvar cancer.

In one aspect, provided herein is a use of an effective amount of a solid form described herein (e.g., any one of Solid Forms 1-12), or any pharmaceutical composition thereof, in the manufacture of a medicament for ameliorating or treating a malignant growth or tumor, wherein the malignant growth or tumor is due to a cancer selected from a breast cancer, a cervical cancer, an ovarian cancer, a uterine cancer, a vaginal cancer, a vulvar cancer, a brain cancer, a cervicocerebral cancer, an esophageal cancer, a thyroid cancer, a small cell cancer, a non-small cell cancer, a lung cancer, a stomach cancer, a gallbladder/bile duct cancer, a liver cancer, a pancreatic cancer, a colon cancer, a rectal cancer, a choriocarcinoma, an uterus body cancer, an uterocervical cancer, a renal pelvis/ureter cancer, a bladder cancer, a prostate cancer, a penis cancer, a testicular cancer, a fetal cancer, a Wilms' cancer, a skin cancer, a malignant melanoma, a neuroblastoma, an osteosarcoma, an Ewing's tumor, a soft part sarcoma, an acute leukemia, a chronic lymphatic leukemia, a chronic myelocytic leukemia, polycythemia vera, a malignant lymphoma, multiple myeloma, a Hodgkin's lymphoma and a non-Hodgkin's lymphoma.

In some embodiments, a cancer is a breast cancer, a cervical cancer, an ovarian cancer, a uterine cancer, a vaginal cancer, or a vulvar cancer.

In one aspect, the provided herein is a solid form described herein (e.g., any one of Solid Forms 1-12), or any pharmaceutical composition thereof, for use in ameliorating or treating a malignant growth or tumor, wherein the malignant growth or tumor is due to a cancer selected from a breast cancer, a cervical cancer, an ovarian cancer, a uterine cancer, a vaginal cancer, a vulvar cancer, a brain cancer, a cervicocerebral cancer, an esophageal cancer, a thyroid cancer, a small cell cancer, a non-small cell cancer, a lung cancer, a stomach cancer, a gallbladder/bile duct cancer, a liver cancer, a pancreatic cancer, a colon cancer, a rectal cancer, a choriocarcinoma, an uterus body cancer, an uterocervical cancer, a renal pelvis/ureter cancer, a bladder cancer, a prostate cancer, a penis cancer, a testicular cancer, a fetal cancer, a Wilms' cancer, a skin cancer, a malignant melanoma, a neuroblastoma, an osteosarcoma, an Ewing's tumor, a soft part sarcoma, an acute leukemia, a chronic lymphatic leukemia, a chronic myelocytic leukemia, polycythemia vera, a malignant lymphoma, multiple myeloma, a Hodgkin's lymphoma and a non-Hodgkin's lymphoma.

In some embodiments, a cancer is a breast cancer, a cervical cancer, an ovarian cancer, a uterine cancer, a vaginal cancer, or a vulvar cancer.

In one aspect, provided herein is a method for inhibiting the activity of WEE1 in a patient in need thereof, said method comprising administering to the patient an effective amount of a solid form described herein (e.g., any one of Solid Forms 1-12), or any pharmaceutical composition thereof.

In one aspect, provided herein is a method of preparing any one of Solid Forms 1-12 of Compound A. In some embodiments, a method of preparing any one of Solid Forms 1-12 of Compound A is substantially as described in Table 2 and/or any one of Examples 2-1 to 2-12.

In one aspect, provided herein is a method of preparing Solid Form 1 of Compound A, said method comprising using a slurry of amorphous Compound A, wherein said slurry comprises the solvents of n-heptane and n-BuOH, and optionally wherein said slurry is heated to about 50° C.

In some embodiments, the ratio of n-heptane and n-BuOH (n-heptane:n-BuOH) is about 8:2.

In some embodiments, Solid Form 1 of Compound A is according to any embodiment described herein.

In one aspect, provided herein is a method of preparing Solid Form 2 of Compound A, said method comprising anti-solvent addition, reverse anti-solvent addition, solid vapor diffusion, liquid vapor diffusion, a slurry at room temperature, a slurry at 50° C., slow evaporation, fast evaporation, slow cooling, and/or temperature cycling.

In some embodiments, ratios of solvents used in methods described herein are understood to be provided as volume:volume (v/v) ratios. For example, a ratio of x:y described herein can be interchangeable with the term x:y (v/v).

In some embodiments, said anti-solvent addition comprises the use of any one of the following solvent systems:

| Combination | Solvent 1 | Solvent 2 |
|---|---|---|
| 1 | Acetone | MTBE |
| 2 | MeCN | MTBE |
| 3 | Pyridine | MTBE |
| 4 | DCM | n-heptane |
| 5 | DCM | MTBE |
| 6 | Dioxane | Toluene |
| 7 | n-PrOH | MTBE |
| 8 | EtOAc | MTBE |
| 9 | IPAC | MTBE |
| 10 | 2-Butanone | MTBE |

In some embodiments, said reverse anti-solvent addition comprises the use of any one of the following solvent systems:

| Combination | Solvent 1 | Solvent 2 |
|---|---|---|
| 1 | DMF | MTBE |
| 2 | Diisopropyl ether | n-heptane |
| 3 | Diisopropyl ether | MTBE |
| 4 | Xylene | MTBE |

In some embodiments, said solvent vapor diffusion comprises the use of diisopropyl ether, MIBK (methyl isobutyl ketone), MeOH, and/or IPAc (isopropyl acetate).

In some embodiments, said liquid vapor diffusion comprises the use of any one of the following solvent systems:

| Combination | Solvent 1 | Solvent 2 |
|---|---|---|
| 1 | DMF | MTBE |
| 2 | n-BuOH | MTBE |
| 3 | EtOH | MTBE |
| 4 | EtOH | Toluene |
| 5 | CHCl₃ | IPAc |
| 6 | CHCl₃ | Toluene |

In some embodiments, said slurry comprises a solvent that is EtOAc and is optionally at room temperature.

In some embodiments, said slurry comprises a solvent system that is n-heptane/xylene, n-heptane/2-MeTHF, or n-heptane/MIBK, and optionally wherein said slurry is at 50° C. and/or the solvents are in a ratio of about 8:2 (n-heptane:xylene, n-heptane:2-MeTHF, or n-heptane:MIBK).

In some embodiments, fast evaporation comprises the use of a solvent that is toluene.

In some embodiments, slow cooling comprises the use of a solvent that is diisopropylether.

In some embodiments, temperature cycling comprises the use of a solvent that is EtOAc or a combination of n-heptane/xylene, and optionally wherein the latter (n-heptane:xylene) is present in a ratio of about 2:1.

In some embodiments, Solid Form 2 of Compound A is according to any embodiment described herein.

In one aspect, provided herein is a method of preparing Solid Form 3 of Compound A, said method comprising using an anti-solvent addition, liquid vapor diffusion, or a slurry at about 50° C.

In some embodiments, anti-solvent addition comprises the use of a solvent combination selected from: n-BuOH/MTBE, dioxane/MTBE, and IPA/MTBE.

In some embodiments, liquid vapor diffusion comprises the use of a solvent system comprising CHCl₃ and cyclohexane.

In some embodiments, a slurry comprises a solvent that is cyclohexane.

In some embodiments, Solid Form 3 of Compound A is according to any embodiment described herein.

In one aspect, provided herein is a method of preparing Solid Form 4 of Compound A, said method comprising using a slurry at about room temperature.

In some embodiments, a slurry comprises a solvent system of heptane and CH₂Cl₂, and optionally wherein said solvents are in a ratio of about 8:2 (heptane:CH₂Cl₂).

In some embodiments, Solid Form 4 of Compound A is according to any embodiment described herein.

In one aspect, provided herein is a method of preparing Solid Form 5 of Compound A, said method comprising using anti-solvent addition, reverse anti-solvent addition, or a slurry at about room temperature.

In some embodiments, anti-solvent addition comprises the use of a solvent system selected from: benzene/MTBE, MeOH/MTBE, and THF/MTBE.

In some embodiments, reverse anti-solvent addition comprises the use of a solvent system that is pyridine/toluene.

In some embodiments, a slurry comprises a solvent system of heptane and dioxane, and optionally wherein said solvents are in a ratio of about 8:2 (heptane:dioxane).

In some embodiments, Solid Form 5 of Compound A is according to any of the embodiments described herein.

In one aspect, provided herein is a method of preparing Solid Form 6 of Compound A, said method comprising using reverse anti-solvent addition or liquid vapor diffusion.

In some embodiments, reverse anti-solvent addition comprises the use of a solvent system that is acetic acid/MTBE or acetic acid/toluene.

In some embodiments, liquid vapor diffusion comprises the use of a solvent system that is acetic acid/MTBE.

In some embodiments, Solid Form 6 of Compound A is according to any of the embodiments described herein.

In one aspect, provided herein is a method of preparing Solid Form 7 of Compound A, said method comprising using reverse anti-solvent addition, liquid vapor diffusion, or polymer induced crystallization.

In some embodiments, reverse anti-solvent addition comprises the use of a solvent system selected from benzene/toluene; n-BuOH/n-heptane; and n-PrOH/n-heptane.

In some embodiments, liquid vapor diffusion comprises the use of a solvent system that is n-BuOH/n-heptane.

In some embodiments, polymer induced crystallization comprises the use of a polymer that is hydroxypropyl methylcellulose (HPMC) and/or a solvent system that is MTBE/IPA, and optionally in a ratio of about 1:1 (MTBE:IPA).

In some embodiments, Solid Form 7 of Compound A is according to any of the embodiments described herein.

In one aspect, provided herein is a method of preparing Solid Form 8 of Compound A, said method comprising using anti-solvent addition or a slurry at room temperature.

In some embodiments, anti-solvent addition comprises the use of a solvent system that is DMAc/n-heptane.

In some embodiments, a slurry comprises a solvent system that is MTBE/DMF, and optionally in a ratio of about 6:4 (MTBE:DMF).

In some embodiments, Solid Form 8 of Compound A is according to any of the embodiments described herein.

In one aspect, provided herein is a method of preparing Solid Form 9 of Compound A, said method comprising using reverse anti-solvent addition.

In some embodiments, reverse anti-solvent addition comprises a solvent system comprising xylene and n-heptane.

In some embodiments, Solid Form 9 of Compound A is according to any of the embodiments described herein.

In one aspect, provided herein is a method of preparing Solid Form 10 of Compound A, said method comprising using slow cooling, and optionally comprising the use of a solvent that is CHCl₃.

In some embodiments, Solid Form 10 of Compound A is according to any of the embodiments described herein.

In one aspect, provided herein is a method of preparing Solid Form 11 of Compound A, said method comprising using sonication induced nucleation, and optionally comprising use of a solvent that is EtOAc.

In some embodiments, Solid Form 11 of Compound A is according to any of the embodiments described herein.

In one aspect, provided herein is a method of preparing Solid Form 12 of Compound A, said method comprising using a slurry at room temperature.

In some embodiments, a slurry comprises a solvent system that is THF/H₂O, and optionally having a water activity (a_{w}) of about 0.2, about 0.4, about 0.6, or about 0.8.

In some embodiments, Solid Form 12 of Compound A is according to any of the embodiments described herein.

In one aspect, provided herein is a pharmaceutical composition comprising an effective amount of a solid form of Compound A described herein (e.g., any one of Solid Forms 1-12), and a pharmaceutically acceptable carrier, diluent, excipient, or combination thereof.

In one aspect, provided herein is a method of preparing any one of Solid Forms 1-12 of Compound A. In some embodiments, a method of preparing any one of Solid Forms 1-12 of Compound A is substantially as described in Table 2 and/or any one of Examples 2-1 to 2-12.

In one aspect, provided herein is a method for ameliorating or treating a malignant growth or tumor in a subject in need thereof, said method comprising administering to the subject an effective amount of a solid form of Compound A described herein (e.g., any one of Solid Forms 1-12), or any pharmaceutical composition thereof. For example, a malignant growth or tumor due to a cancer described herein.

In one aspect, provided herein is a method for ameliorating or treating a cancer in a subject in need thereof, said method comprising administering to the subject an effective amount of a solid form of Compound A described herein (e.g., any one of Solid Forms 1-12), or any pharmaceutical composition thereof.

In one aspect, provided herein is a method for inhibiting the activity of WEE1 in a subject in need thereof, said method comprising administering to the subject an effective amount of a solid form described herein (e.g., any one of Solid Forms 1-12), or any pharmaceutical composition thereof. In one aspect, provided herein is a method for inhibiting the activity of WEE1 using an effective amount of a solid form described herein (e.g., any one of Solid Forms 1-12), or any pharmaceutical composition thereof.

In some embodiments, provided herein is a pharmaceutical composition that can include an effective amount of a solid form of Compound A, including those described herein (for example, Solid Forms 1-12), and a pharmaceutically acceptable carrier, diluent, excipient, or combination thereof.

In some embodiments, provided herein is the use of a solid form of Compound A (for example, Solid Form 1 of Compound A, Solid Form 2 of Compound A, Solid Form 3 of Compound A, Solid Form 4 of Compound A, Solid Form 5 of Compound A, Solid Form 6 of Compound A, Solid Form 7 of Compound A, Solid Form 8 of Compound A, Solid Form 9 of Compound A, Solid Form 10 of Compound A, Solid Form 11 of Compound A, and/or Solid Form 12 of Compound A), or a pharmaceutical composition that can include an effective amount of a solid form of Compound A, (such as those described herein, for use in the manufacture of a medicament for ameliorating or treating a malignant growth or tumor.

In some embodiments, the malignant growth or tumor can be due to a cancer selected from a breast cancer, a cervical cancer, an ovarian cancer, a uterine cancer, a vaginal cancer, a vulvar cancer, a brain cancer, a cervicocerebral cancer, an esophageal cancer, a thyroid cancer, a small cell cancer, a non-small cell cancer, a lung cancer, a stomach cancer, a gallbladder/bile duct cancer, a liver cancer, a pancreatic cancer, a colon cancer, a rectal cancer, a choriocarcinoma, an uterus body cancer, an uterocervical cancer, a renal pelvis/ureter cancer, a bladder cancer, a prostate cancer, a penis cancer, a testicular cancer, a fetal cancer, a Wilms' cancer, a skin cancer, a malignant melanoma, a neuroblastoma, an osteosarcoma, an Ewing's tumor, a soft part sarcoma, an acute leukemia, a chronic lymphatic leukemia, a chronic myelocytic leukemia, polycythemia vera, a malignant lymphoma, multiple myeloma, a Hodgkin's lymphoma and a non-Hodgkin's lymphoma.

### BRIEF DESCRIPTION OF DRAWINGS

FIG. 1A illustrates a representative XRPD pattern of Solid Form 1 of Compound A.

FIG. 1B illustrates a representative DSC and TGA thermograms of Solid Form 1 of Compound A.

FIG. 2A illustrates a representative XRPD pattern of Solid Form 2 of Compound A.

FIG. 2B illustrates a representative DSC and TGA thermograms of Solid Form 2 of Compound A.

FIG. 3A illustrates a representative XRPD pattern of Solid Form 3 of Compound A.

FIG. 3B illustrates a representative DSC and TGA thermograms of Solid Form 3 of Compound A.

FIG. 4 illustrates a representative XRPD pattern of Solid Form 4 of Compound A.

FIG. 5A illustrates a representative XRPD pattern of Solid Form 5 of Compound A.

FIG. 5B illustrates a representative DSC and TGA thermograms of Solid Form 5 of Compound A.

FIG. 6A illustrates a representative XRPD pattern of Solid Form 6 of Compound A.

FIG. 6B illustrates a representative DSC and TGA thermograms of Solid Form 6 of Compound A.

FIG. 7A illustrates a representative XRPD pattern of Solid Form 7 of Compound A.

FIG. 7B illustrates a representative DSC and TGA thermograms of Solid Form 7 of Compound A.

FIG. 8A illustrates a representative XRPD pattern of Solid Form 8 of Compound A.

FIG. 8B illustrates a representative DSC and TGA thermograms of Solid Form 8 of Compound A.

FIG. 9A illustrates a representative XRPD pattern of Solid Form 9 of Compound A.

FIG. 9B illustrates a representative DSC and TGA thermograms of Solid Form 9 of Compound A.

FIG. 10 illustrates a representative XRPD pattern of Solid Form 10 of Compound A.

FIG. 11A illustrates a representative XRPD pattern of Solid Form 11 of Compound A.

FIG. 11B illustrates a representative DSC and TGA thermograms of Solid Form 11 of Compound A.

FIG. 12A illustrates a representative XRPD pattern of Solid Form 12 of Compound A.

FIG. 12B illustrates a representative DSC and TGA thermograms of Solid Form 12 of Compound A.

FIG. 13A illustrates a representative XRPD pattern of Form E of Compound A.

FIG. 13B illustrates a representative DSC and TGA thermograms of Form E of Compound A.

FIG. 13C illustrates a representative PLM image of Form E of Compound A.

FIG. 14 illustrates a representative ¹H NMR spectrum in DMSO-d₆ of Compound A.

FIG. 15 illustrates a representative XRPD pattern of an amorphous form of Compound A.

### DETAILED DESCRIPTION

WEE1 is a tyrosine kinase that is a critical component of the ATR-mediated G2 cell cycle checkpoint control that prevents entry into mitosis in response to cellular DNA damage. ATR phosphorylates and activates CHK1, which in turn activates WEE1, leading to the selective phosphorylation of cyclin-dependent kinase 1 (CDK1) at Tyr15, thereby stabilizing the CDK1-cyclin B complex and halting cell-cycle progression. This process confers a survival advantage by allowing tumor cells time to repair damaged DNA prior to entering mitosis. Inhibition of WEE1 abrogates the G2 checkpoint, promoting cancer cells with DNA damage to enter into unscheduled mitosis and undergo cell death via mitotic catastrophe. Therefore, WEE1 inhibition has the potential to sensitize tumors to DNA-damaging agents, such as cisplatin, and to induce tumor cell death.

Described herein are new solid forms of the WEE1 inhibitor (R)-2-allyl-1-(7-ethyl-7-hydroxy-6,7-dihydro-5H-cyclopenta[b]pyridin-2-yl)-6-((4-(4-methylpiperazin-1-yl)phenyl)amino)-1,2-dihydro-3H-pyrazolo[3,4-d]pyrimidin-3-one (Compound A),

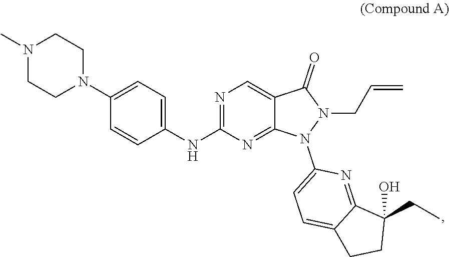 [SMILES: C=CCn1c(=O)c2cnc(Nc3ccc(N4CCN(C)CC4)cc3)nc2n1-c1ccc2c(n1)[C@@](O)(CC)CC2]

or a pharmaceutically acceptable salt thereof. Solid forms described herein can have beneficial properties, including as compared to other solid forms of Compound A.

### I. Definitions

Unless defined otherwise, all technical and scientific terms used herein have the same meaning as is commonly understood by one of ordinary skill in the art. All patents, applications, published applications and other publications referenced herein are incorporated by reference in their entirety unless stated otherwise. In the event that there are a plurality of definitions for a term herein, those in this section prevail unless stated otherwise.

Unless otherwise specified, the term “crystalline” and related terms used herein, when used to describe a substance, component, product or form, mean that the substance, component, product or form is substantially crystalline, for example, as determined by X-ray diffraction. (see, e.g., Remington's Pharmaceutical Sciences, 20^{th} ed., Lippincott Williams & Wilkins, Philadelphia Pa., 173 (2000); The United States Pharmacopeia, 37^{th} ed., 503-509 (2014)).

The terms “crystal form” and “crystalline form” refer to a crystalline solid form comprising a chemical compound, and may refer to a particular single-component or multiple-component crystal form, including, but not limited to, a polymorph, a solvate, a hydrate or other molecular complex, a salt, a solvate of a salt, a hydrate of a salt, or other molecular complex of a salt, or a polymorph thereof.

The terms “polymorphs” and “polymorphic forms” refer to two or more crystal forms that comprise the same molecule, molecules or ions. Different polymorphs may have different physical properties such as, for example, melting temperatures, heats of fusion, solubilities, dissolution rates and/or vibrational spectra as a result of the arrangement or conformation of the molecules or ions in the crystal lattice. The differences in physical properties exhibited by polymorphs affect pharmaceutical parameters such as storage stability, compressibility and density (important in formulation and product manufacturing), and/or dissolution rate (an important factor in bioavailability). Differences in stability can result from changes in chemical reactivity (e.g., differential oxidation, such that a dosage form discolors more rapidly when comprised of one polymorph than when comprised of another polymorph) or mechanical changes (e.g., tablets crumble on storage as a kinetically favored polymorph converts to thermodynamically more stable polymorph) or both (e.g., tablets of one polymorph are more susceptible to breakdown at high humidity). As a result of solubility/dissolution differences, in the extreme case, some polymorphic transitions may result in lack of potency or, at the other extreme, toxicity. In addition, the physical properties of the crystal may be important in processing; for example, one polymorph might be more likely to form solvates or might be difficult to filter and wash free of impurities (e.g., particle shape and size distribution might be different between polymorphs).

As used herein, and unless otherwise specified, the terms “about” and “approximately,” when used in connection with doses, amounts or weight percents of ingredients of a composition or a dosage form, mean a dose, amount or weight percent that is recognized by one of ordinary skill in the art to provide a pharmacological effect equivalent to that obtained from the specified dose, amount, or weight percent. In some embodiments, the terms “about” and “approximately,” when used in this context, contemplate a dose, amount, or weight percent within 30%, within 20%, within 15%, within 10%, or within 5%, of the specified dose, amount or weight percent.

As used herein, and unless otherwise specified, the terms “about” and “approximately,” when used in connection with a numeric value or range of values which is provided to characterize a particular solid form, e.g., a specific temperature or temperature range (for example, that describes a melting, dehydration, desolvation or glass transition temperature); a mass change (for example, a mass change as a function of temperature or humidity); a solvent or water content (for example, mass or a percentage); or a peak position (for example, in analysis by, for example, IR or Raman spectroscopy or XRPD); indicate that the value or range of values may deviate to an extent deemed reasonable to one of ordinary skill in the art while still describing the solid form. Techniques for characterizing crystal forms and amorphous forms include, but are not limited to, thermal gravimetric analysis (TGA), differential scanning calorimetry (DSC), X-ray powder diffractometry (XRPD), single-crystal X-ray diffractometry, vibrational spectroscopy, e.g., infrared (IR) and Raman spectroscopy, solid-state and solution nuclear magnetic resonance (NMR) spectroscopy, optical microscopy, hot stage optical microscopy, scanning electron microscopy (SEM), electron crystallography and quantitative analysis, particle size analysis (PSA), surface area analysis, solubility studies and dissolution studies. In some embodiments, the terms “about” and “approximately,” when used in this context, indicate that the numeric value or range of values may vary within 30%, 20%, 15%, 10%, 9%, 8%, 7%, 6%, 5%, 4%, 3%, 2%, 1.5%, 1%, 0.5%, or 0.25% of the recited value or range of values. In the context of molar ratios, “about” and “approximately” indicate that the numeric value or range of values may vary within 20%, 15%, 10%, 9%, 8%, 7%, 6%, 5%, 4%, 3%, 2%, 1.5%, 1%, 0.5%, or 0.25% of the recited value or range of values. It should be understood that the numerical values of the peaks of an X-ray powder diffraction pattern may vary from one machine to another, or from one sample to another, and so the values quoted are not to be construed as absolute, but with an allowable variability, such as ±0.2 degrees two theta (° 2θ), or more. For example, in some embodiments, the value of an XRPD peak position may vary by up to ±0.2 degrees 2θ while still describing the particular XRPD peak.

As used herein, and unless otherwise specified, a solid form that is “substantially physically pure” is substantially free from other solid forms, such Solid Forms 1-12. In some embodiments, a crystal form that is substantially physically pure contains less than about 50%, 45%, 40%, 35%, 30%, 25%, 20%, 15%, 10%, 9%, 8%, 7%, 6%, 5%, 4%, 3%, 2%, 1%, 0.5%, 0.4%, 0.3%, 0.2%, 0.1%, 0.05%, or 0.01% of one or more other solid forms on a weight basis. The detection of other solid forms can be accomplished by any method apparent to a person of ordinary skill in the art, including, but not limited to, diffraction analysis, thermal analysis, elemental combustion analysis and/or spectroscopic analysis.

As used herein, and unless otherwise specified, a solid form that is “substantially chemically pure” is substantially free from other chemical compounds (i.e., chemical impurities). In some embodiments, a solid form that is substantially chemically pure contains less than about 50%, 45%, 40%, 35%, 30%, 25%, 20%, 15%, 10%, 9%, 8%, 7%, 6%, 5%, 4%, 3%, 2%, 1%, 0.5%, 0.4%, 0.3%, 0.2%, 0.1%, 0.05%, or 0.01% of one or more other chemical compounds on a weight basis. The detection of other chemical compounds can be accomplished by any method apparent to a person of ordinary skill in the art, including, but not limited to, methods of chemical analysis, such as, e.g., mass spectrometry analysis, spectroscopic analysis, thermal analysis, elemental combustion analysis and/or chromatographic analysis.

As used herein, and unless otherwise indicated, a chemical compound, solid form, or composition that is “substantially free” of another chemical compound, solid form, or composition means that the compound, solid form, or composition contains less than about 50%, 45%, 40%, 35%, 30%, 25%, 20%, 15%, 10%, 9%, 8%, 7%, 6%, 5%, 4%, 3%, 2%, 1%, 0.5%, 0.4%, 0.3%, 0.2% 0.1%, 0.05%, or 0.01% by weight of the other compound, solid form, or composition.

It is understood that, in any compound described herein having one or more chiral centers, if an absolute stereochemistry is not expressly indicated, then each center may independently be of R-configuration or S-configuration or a mixture thereof. Thus, the compounds provided herein may be enantiomerically pure, enantiomerically enriched, racemic mixture, diastereomerically pure, diastereomerically enriched, or a stereoisomeric mixture. In addition, it is understood that, in any compound described herein having one or more double bond(s) generating geometrical isomers that can be defined as E or Z, each double bond may independently be E or Z a mixture thereof. Likewise, it is understood that, in any compound described, all tautomeric forms are also intended to be included.

It is understood that the compounds described herein can be labeled isotopically. Substitution with isotopes such as deuterium may afford certain therapeutic advantages resulting from greater metabolic stability, such as, for example, increased in vivo half-life or reduced dosage requirements. Each chemical element as represented in a compound structure may include any isotope of said element. For example, in a compound structure a hydrogen atom may be explicitly disclosed or understood to be present in the compound. At any position of the compound that a hydrogen atom may be present, the hydrogen atom can be any isotope of hydrogen, including but not limited to hydrogen-1 (protium) and hydrogen-2 (deuterium). Thus, reference herein to a compound encompasses all potential isotopic forms unless the context clearly dictates otherwise.

Where a range of values is provided, it is understood that the upper and lower limit, and each intervening value between the upper and lower limit of the range is encompassed within the embodiments.

Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like; the term ‘comprising’ as used herein is synonymous with ‘including,’ ‘containing,’ or ‘characterized by,’ and is inclusive or open-ended and does not exclude additional, unrecited elements or method steps; the term ‘having’ should be interpreted as ‘having at least;’ the term ‘includes’ should be interpreted as ‘includes but is not limited to;’ the term ‘example’ is used to provide exemplary instances of the item in discussion, not an exhaustive or limiting list thereof; and use of terms like ‘preferably,’ ‘preferred,’ ‘desired,’ or ‘desirable,’ and words of similar meaning should not be understood as implying that certain features are critical, essential, or even important to the structure or function, but instead as merely intended to highlight alternative or additional features that may or may not be utilized in a particular embodiment. In addition, the term “comprising” is to be interpreted synonymously with the phrases “having at least” or “including at least”. When used in the context of a process, the term “comprising” means that the process includes at least the recited steps, but may include additional steps. When used in the context of a compound, composition or device, the term “comprising” means that the compound, composition or device includes at least the recited features or components, but may also include additional features or components.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The indefinite article “a” or “an” does not exclude a plurality. The mere fact that certain measures are recited in mutually different dependent claims does not indicate that a combination of these measures cannot be used to advantage. Any reference signs in the claims should not be construed as limiting the scope.

“Solvent” can include, but is not limited to, non-polar, polar aprotic, and polar protic solvents. Illustrative examples of non-polar solvents include, but are not limited to, pentane, cyclopentane, hexane, cyclohexane, benzene, toluene, 1,4-dioxane, chloroform, diethyl ether, and dichloromethane (DCM). Illustrative examples of polar aprotic solvents include, but are not limited to, tetrahydrofuran (THF), ethyl acetate, acetone, dimethylformamide (DMF), acetonitrile (MeCN), dimethyl sulfoxide (DMSO), nitromethane, and propylene carbonate. Illustrative examples of polar protic solvents include, but are not limited to, formic acid, n-butanol, isopropanol (IPA), n-propanol, ethanol, methanol, acetic acid, and water.

“Solvate” can include, but is not limited to, a solvate that retains one or more of the activities and/or properties of the compound and that is not undesirable. Examples of solvates include, but are not limited to, a compound in combination with water, isopropanol, ethanol, methanol, DMSO, ethyl acetate, acetic acid, ethanolamine, or combinations thereof.

### II. Solid Forms of Compound A

Certain embodiments herein provide single-component and multiple-component (e.g., pharmaceutically acceptable salts, solvates, and/or hydrates) solid forms of (R)-2-allyl-1-(7-ethyl-7-hydroxy-6,7-dihydro-5H-cyclopenta[b]pyridin-2-yl)-6-((4-(4-methylpiperazin-1-yl)phenyl)amino)-1,2-dihydro-3H-pyrazolo[3,4-d]pyrimidin-3-one (Compound A), having the chemical structure:

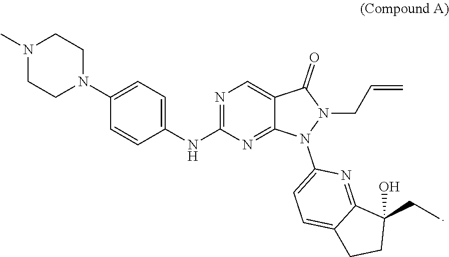 [SMILES: C=CCn1c(=O)c2cnc(Nc3ccc(N4CCN(C)CC4)cc3)nc2n1-c1ccc2c(n1)[C@@](O)(CC)CC2]

Compound A is also referred to herein as the “freebase of Compound A.” If there is an inconsistency between the name of Compound A and a structure of Compound A provided herein, then the structure of Compound A in this paragraph is what is meant for Compound A.

Solid forms described herein can be prepared from other forms of Compound A, including amorphous forms of Compound A.

In some embodiments, an amorphous form of Compound A can be obtained from the freebase Form E of Compound A. Exemplary methods are described herein.

In some embodiments, a variety of polymorphs of Compound A can be obtained from an amorphous form of Compound A. Some embodiments disclosed herein relate to freebase Form 1 of Compound A. Some embodiments disclosed herein relate to freebase Form 2 of Compound A. Some embodiments disclosed herein relate to freebase Form 3 of Compound A. Some embodiments disclosed herein relate to freebase Form 4 of Compound A. Some embodiments disclosed herein relate to freebase Form 5 of Compound A. Some embodiments disclosed herein relate to freebase Form 6 of Compound A. Some embodiments disclosed herein relate to freebase Form 7 of Compound A. Some embodiments disclosed herein relate to freebase Form 8 of Compound A. Some embodiments disclosed herein relate to freebase Form 9 of Compound A. Some embodiments disclosed herein relate to freebase Form 10 of Compound A. Some embodiments disclosed herein relate to freebase Form 11 of Compound A. Some embodiments disclosed herein relate to freebase Form 12 of Compound A.

In some embodiments, a freebase described herein can further include one or more other polymorph forms. For example, freebase Form 1 may further include freebase Form E and/or one of more of other freebase forms. In some embodiments, freebase Form 2 may further include freebase Form E and/or one of more of other freebase forms. In some embodiments, freebase Form 3 may further include freebase Form E and/or one of more of other freebase forms. In some embodiments, freebase Form 4 may further include freebase Form E and/or one of more of other freebase forms. In some embodiments, freebase Form 5 may further include freebase Form E and/or one of more of other freebase forms. In some embodiments, freebase Form 6 may further include freebase Form E and/or one of more of other freebase forms. In some embodiments, freebase Form 7 may further include freebase Form E and/or one of more of other freebase forms. In some embodiments, freebase Form 8 may further include freebase Form E and/or one or more of other freebase forms. In some embodiments, freebase Form 9 may further include freebase Form E and/or one of more of other freebase forms. In some embodiments, freebase Form 10 may further include freebase Form E and/or one of more of other freebase forms. In some embodiments, freebase Form 11 may further include freebase Form E and/or one of more of other freebase forms. In some embodiments, freebase Form 12 may further include freebase Form E and/or one of more of other freebase forms.

In a freebase form of Compound A, various amounts of freebase forms of Compound A can be present. For example, the amount of freebase of Compound A that can be present in Form 1 can be in the range of about 90% to 100%. In some embodiments, the amount of freebase of Compound A that can be present in Form 1 can be in the range of about 95% to 100%. In other embodiments, the amount of freebase of Compound A that can be present in Form 1 can be in the range of about 97% to 100%. In other embodiments, the amount of freebase of Compound A that can be present in Form 1 can be in the range of about 98% to 100%. In other embodiments, the amount of freebase of Compound A that can be present in Form 1 can be in the range of about 99% to 100%.

In some embodiments, a solid form described herein (such as Solid Forms 1-12) is amorphous.

In other embodiments, a solid form described herein (for example, Solid Forms 1-12) is crystalline.

In some embodiments, a solid form described herein is substantially free from other solid forms (e.g., substantially free of any other crystalline forms of Compound A and/or any amorphous form of Compound A). In some embodiments, a solid form of Compound A (e.g., any of Solid Forms 1-12 as described herein) that is substantially free from other solid forms of Compound A contains less than about 50%, 45%, 40%, 35%, 30%, 25%, 20%, 15%, 10%, 9%, 8%, 7%, 6%, 5%, 4%, 3%, 2%, 1%, 0.5%, 0.4%, 0.3%, 0.2%, 0.1%, 0.05%, or 0.01% of one or more other solid forms (such as Solid Forms 1-12) on a weight:weight (wt:wt) basis. The detection of other solid forms can be accomplished by any method apparent to a person of ordinary skill in the art, including, but not limited to, diffraction analysis, thermal analysis, elemental combustion analysis and/or spectroscopic analysis.

### III. Polymorphism

The ability of a substance to exist in more than one crystal form is defined as polymorphism; the different crystal forms of a particular substance are referred to as “polymorphs.” In general, polymorphism is affected by the ability of a molecule of a substance to change its conformation or to form different intermolecular or intra-molecular interactions, particularly hydrogen bonds, which is reflected in different atom arrangements in the crystal lattices of different polymorphs. In contrast, the overall external form of a substance is known as “morphology,” which refers to the external shape of the crystal and the planes present, without reference to the internal structure. Crystals can display different morphology based on different conditions, such as, for example, growth rate, stirring, and the presence of impurities.

The different polymorphs of a substance can possess different energies of the crystal lattice and, thus, in solid state they can show different physical properties such as form, density, melting point, color, stability, solubility, dissolution rate, etc., which can, in turn, affect the stability, dissolution rate, and/or bioavailability of a given polymorph and its suitability for use as a pharmaceutical and in pharmaceutical compositions. Different polymorphs of a compound (e.g., Compound A) can incorporate different impurities, or chemical residues, upon crystallization. Certain polymorphs incorporate very little, or no, chemical residues. Accordingly, the formation of certain polymorph forms of a compound may result in purification of the compound.

### IV. Characterization of Solid Forms and Polymorphs of Compound A

### Characterization Methods

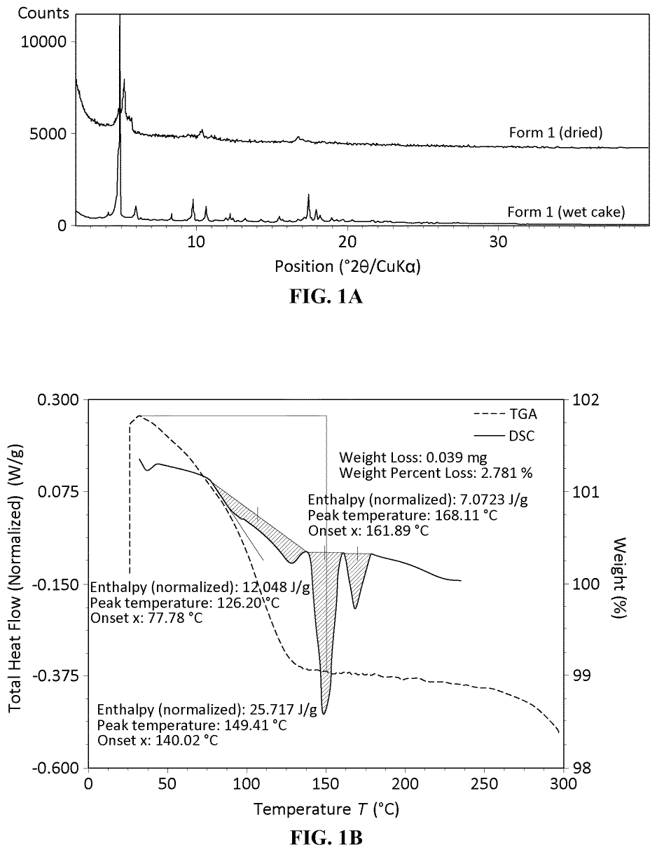

Various methods known in the art can be used to characterize a solid forms such as those described herein. For example, characterization techniques can include X-ray diffraction (e.g., XRPD), DSC, TGA, IR, TGIR, ¹H NMR and/or ¹³C NMR. The working examples described herein illustrate a variety of polymorph screening and characterization techniques used to identify various solids forms of Compound A. For example, XRPD, DSC and/or TGA were used to characterize the amorphous and polymorphic crystalline forms illustrated in FIG. 1-15 and summarized in Table 2.

The terms “form” and “pattern” may be used interchangeably herein to refer to a solid form characterized by a corresponding XRPD pattern e.g., “Form 1” may be used interchangeably with “Pattern 1” and vice-versa.

### 1. Powder X-Ray Diffraction (PXRD or XRPD)

XRPD data was collected using Panalytical Empyrean. Parameters setting are described in Table 1.

TABLE 1
Parameters for XRPD

| | X-Ray tube Cu |
|---|---|
| | (Kα radiation); |
| | tube voltage 45 kV; |
| Parameters | tube current 40 mA |
| Scanning range [°2θ] | 2 to 40 |
| Step size [°2θ] | 6.33 [°2θ]/min |
| Scan type | Continuous |

### 2. Proton Nuclear Magnetic Resonance (¹H NMR)

¹H NMR data was collected using Bruker 400 Ultrashield and deuterated solvent such as dimethyl sulfoxide-d6 (DMSO-d₆).

3. Differential Scanning calorimetry (DSC)

DSC data were collected using a TA Instruments DSC 2500. Parameters include Ramp 10° C. per minute, up to 250° C.

### 4. Thermo-Gravimetric Analysis (TGA)

TGA data were collected using TA Instruments TGA 5500. Parameters include 25 to 300° C., 10° C./min, and N₂ sweep.

### 5. Polarized Light Microscopy (PLM)

PLM data were collected using Nikon Eclipse Ci Pol and a software such as Nikon NIS Elements.

Furthermore, although the foregoing has been described in some detail by way of illustrations and examples for purposes of clarity and understanding, it will be understood by those of skill in the art that numerous and various modifications can be made without departing from the spirit of the present disclosure. Therefore, it should be clearly understood that the forms disclosed herein are illustrative only and are not intended to limit the scope of the present disclosure, but rather to also cover all modification and alternatives coming with the true scope and spirit of the present disclosure.

In some embodiments, solid forms of Compound A are identifiable on the basis of peaks in an X-ray powder diffraction analysis. X-ray powder diffraction, also referred to as XRPD or PXRD, is a scientific technique using X-ray, neutron, or electron diffraction on powder, microcrystalline, or other solid materials for structural characterization of the materials.

In some embodiments, solid forms of Compound A may be defined by their differential scanning calorimetry (DSC) thermograms. The DSC thermogram is used to observe crystallization events, and, in particular, measure the crystallization temperature (Tc) of a sample via a characteristic endotherm.

In some embodiments, solid forms of Compound A may be defined by thermal gravimetric analysis (TGA), which is a method of thermal analysis in which the mass of a sample is measured over time. This measurement provides information about physical phenomena, such as phase transitions, absorption, and desorption; as well as chemical phenomena including chemisorption, thermal decomposition, and solid-gas reactions (e.g., oxidation or reduction).

Exemplary solid forms are summarized in Table 2, along with exemplary XRPD, DSC and/or TGA characteristics, which are also further illustrated in FIGS. 1-12.

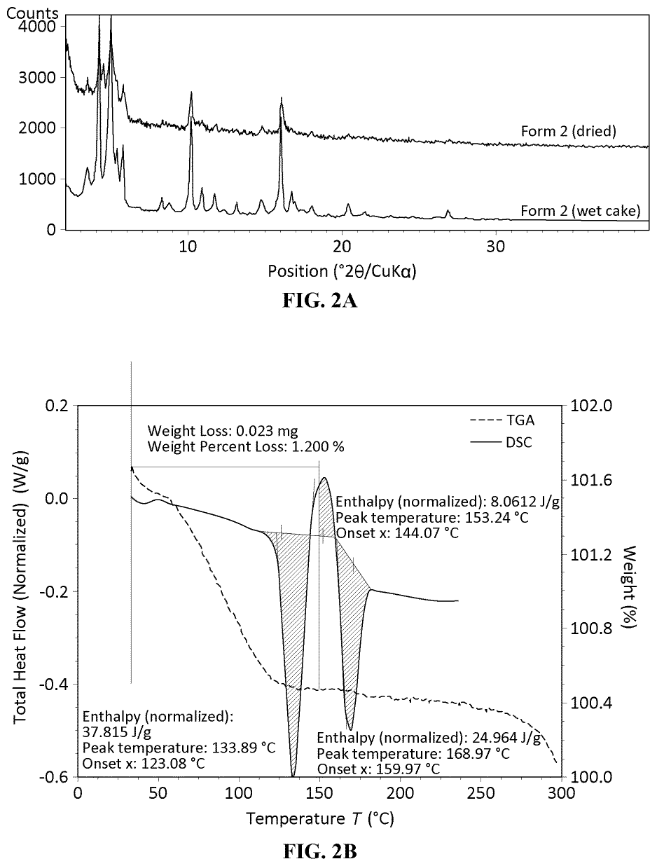

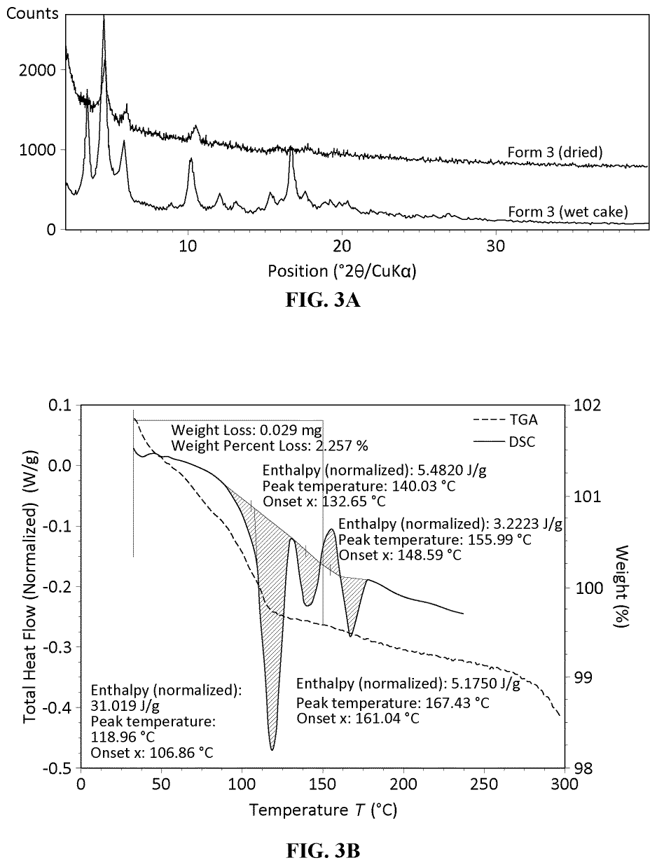

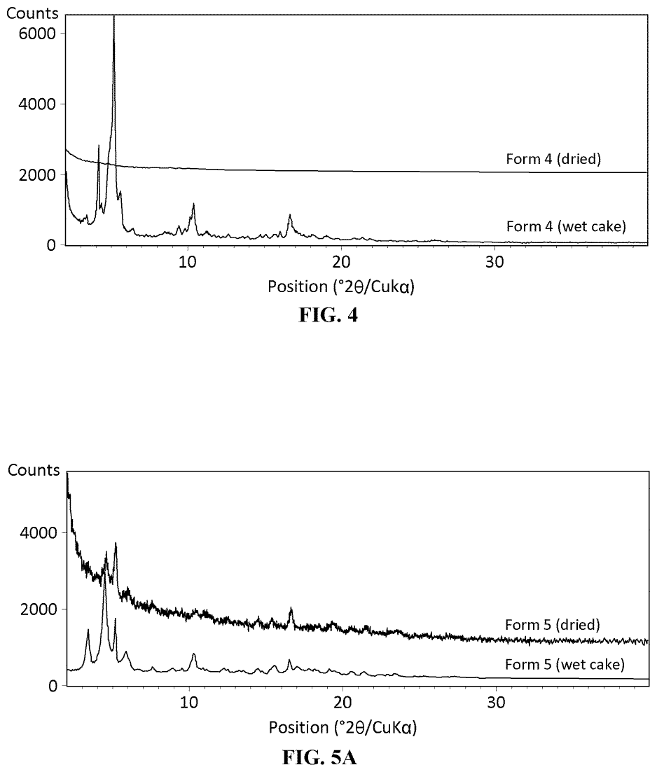

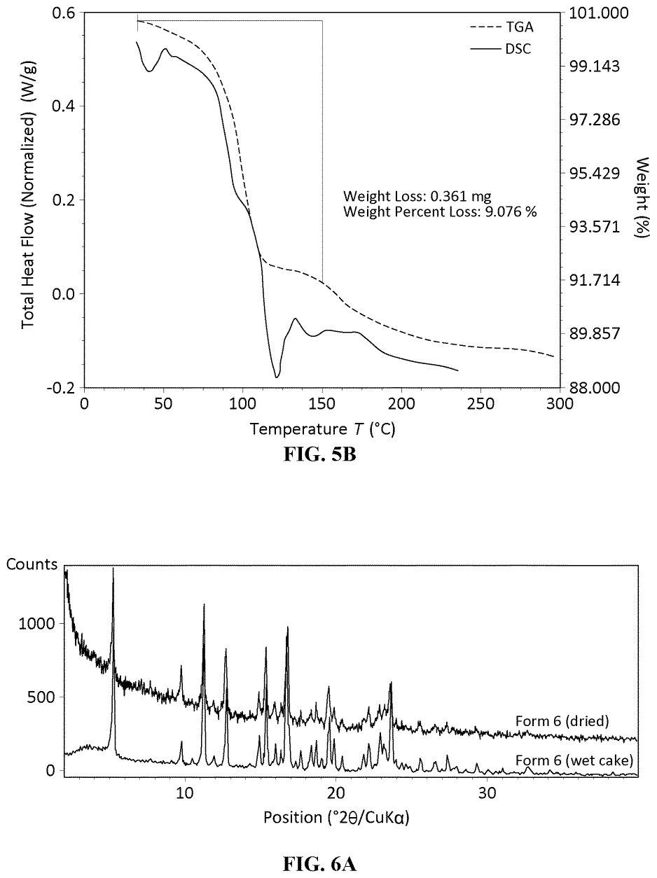

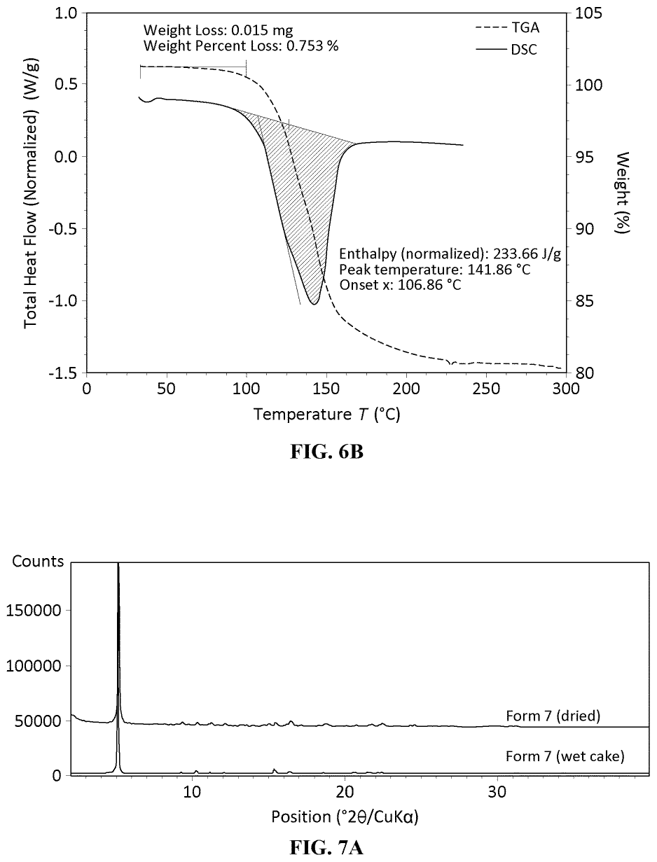

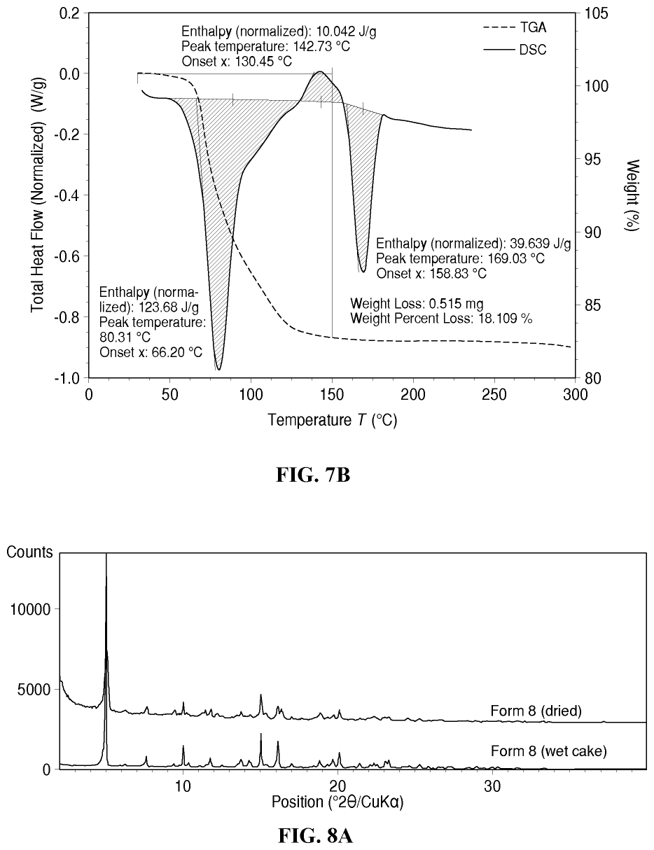

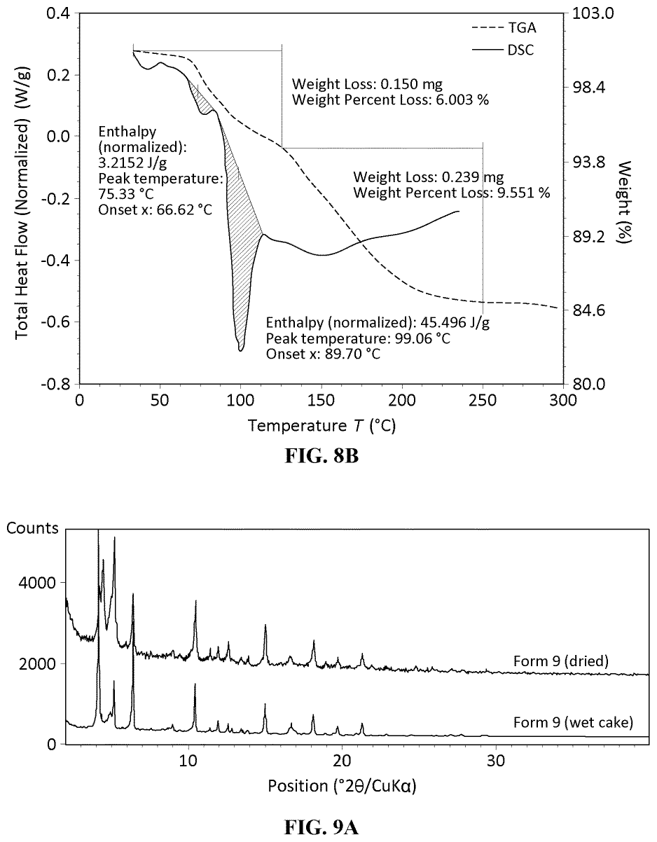

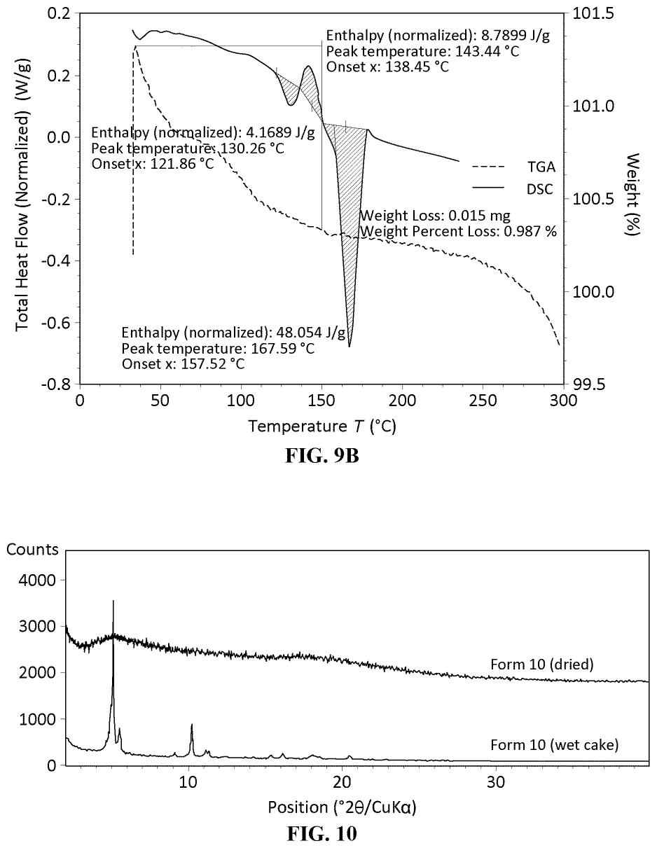

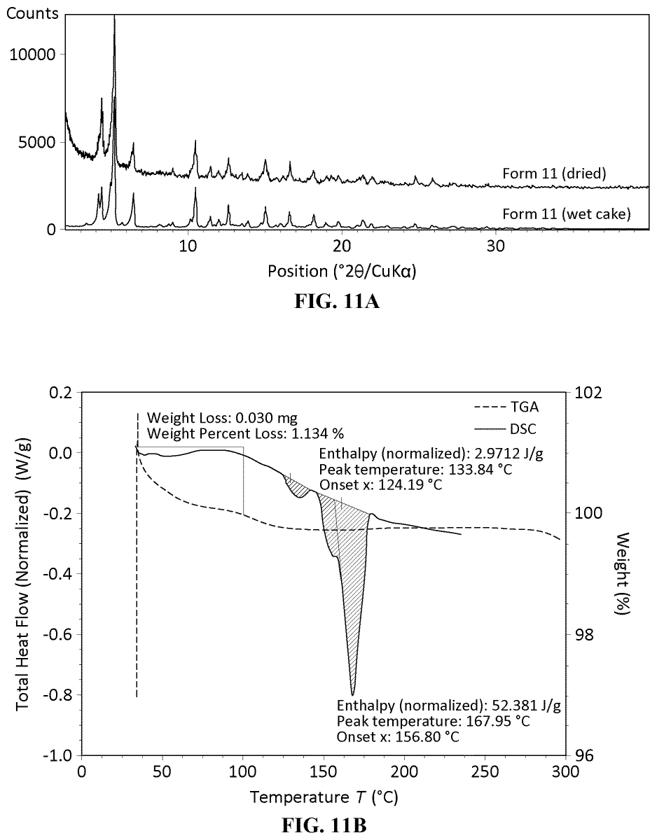

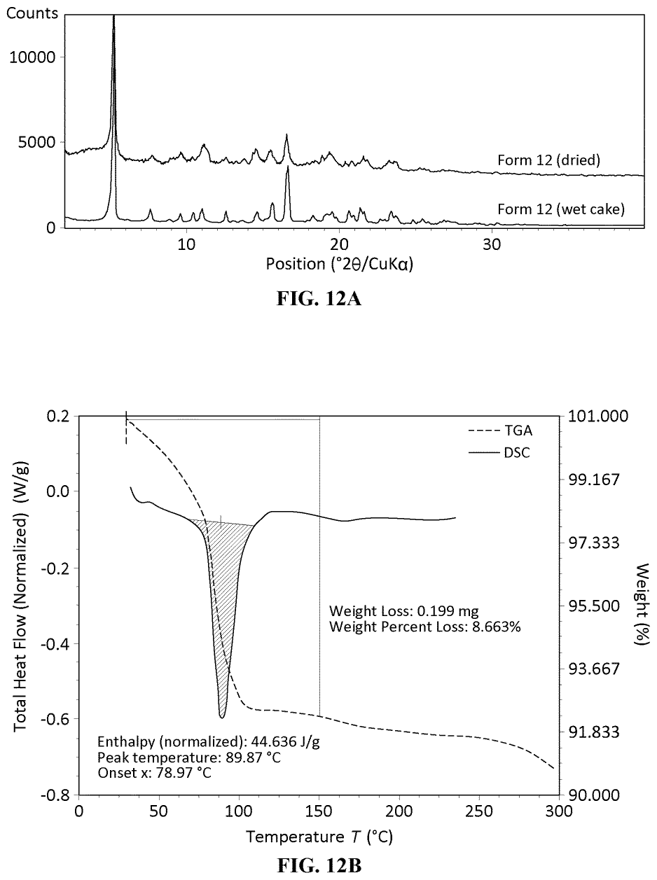

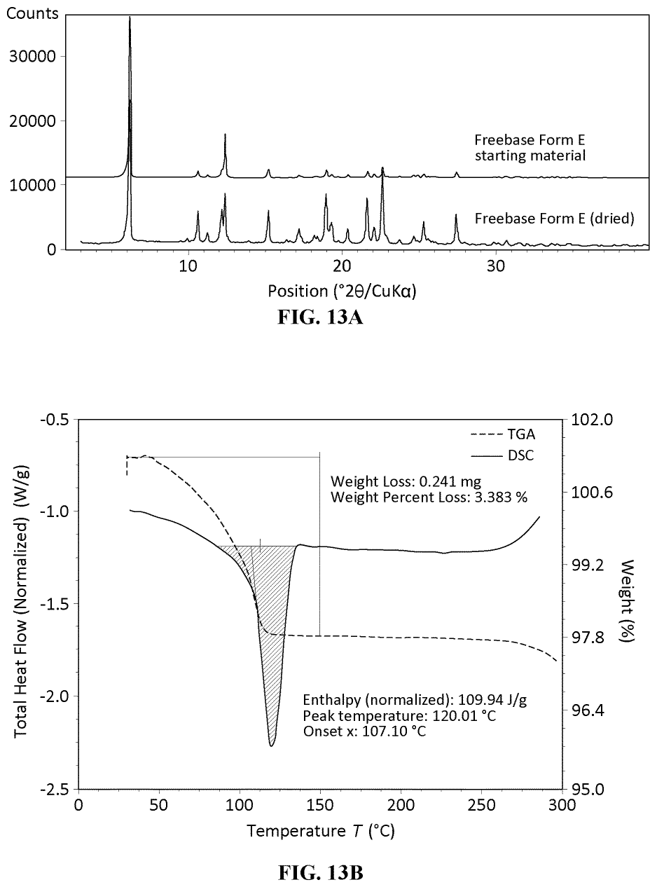

TABLE 2
Exemplary Preparation and Characteristics of Solid Forms of Compound A

| Solid Form | Method for Preparation | | XRPD | DSC (Tpeak) | | TGA wt. % |
|---|---|---|---|---|---|---|
| Form 1 | Slurry at 50° C. | | FIG. 1A | 1^{st} endo: | 126.2° C. | 2.78% |
| | n-heptane/n-BuOH | (8:2) | | 2^{nd} endo: | 149.4° C. | (RT-150° C.) |
| | | | | 3^{rd} endo: | 168.1° C. | FIG. 1B |
| | | | | FIG. 1B | | |
| Form 2 | Slurry at 50° C. | | FIG. 2A | 1^{st} endo: | 133.9° C. | 1.20% |
| | n-heptane/xylene | (8:2) | | 1^{st} exo: | 153.2° C. | (RT-150° C.) |

| | | | 2^{nd} endo: | 169.0° C. | FIG. 2B |
|---|---|---|---|---|---|
| | | | FIG. 2B | | |
| Form 3 | Slurry at 50° C. | FIG. 3A | 1^{st} endo: | 119.0° C. | 2.26% |
| | Cyclohexane | | 2^{nd} endo: | 140.0° C. | (RT-150° C.) |
| | | | 1^{st} exo: | 156.0° C. | FIG. 3B |
| | | | 3^{rd} endo: | 167.4° C. | |

| | | | | FIG. 3B | |
|---|---|---|---|---|---|
| Form 4 | Slurry at RT | | FIG. 4 | N/A | N/A |
| | n-heptane/DCM | (8:2) | | | |
| Form 5 | Slurry at RT | | FIG. 5A | FIG. 5B | 9.08% |
| | n-heptane/dioxane | (8:2) | | | (RT-150° C.) |

| | | | | | FIG. 5B |
|---|---|---|---|---|---|
| Form 6 | Reverse anti-solvent | FIG. 6A | 1^{st} endo: | 141.9° C. | 0.75% |
| | Addition | | FIG. 6B | | (RT-100° C.) |
| | Acetic acid/MTBE | | | | FIG. 6B |
| Form 7 | Reverse anti-solvent | FIG. 7A | 1^{st} endo: | 80.3° C. | 18.1% |
| | Addition | | 1^{st} exo: | 142.7° C. | (RT-150° C.) |
| | n-BuOH/n-heptane | | 2^{nd} endo: | 169.0° C. | FIG. 7B |
| | | | FIG. 7B | | |
| Form 8 | Reverse anti-solvent | FIG. 8A | 1^{st} endo: | 75.3° C. | 1st: 6.0% |
| | Addition | | 2^{nd} endo: | 99.1° C. | (RT-125° C.) |
| | DMAc/n-heptane | | FIG. 8B | | 2nd: 9.55% |
| | | | | | (125.01-252° C.) |
| | | | | | FIG. 8B |
| Form 9 | Reverse anti-solvent | FIG. 9A | 1^{st} endo: | 130.3° C. | 0.99% |
| | Addition | | 1^{st} exo: | 143.4° C. | (RT-150° C.) |
| | Xylene/n-heptane | | 2^{nd} endo: | 167.6° C. | FIG. 9B |
| | | | FIG. 9B | | |
| Form 10 | Slow Cooling | FIG. 10 | N/A | | N/A |
| | CHCl₃ (SE) | | | | |
| Form 11 | Sonication induced | FIG. 11A | 1^{st} endo: | 133.8° C. | 1.13% |
| | EtOAc | | 2^{nd} endo: | 168.0° C. | (RT-100° C.) |
| | | | FIG. 11B | | FIG. 11B |
| Form 12 | Slurry at RT | FIG. 12A | 1^{st} endo: | 89.9° C. | 8.66% |

| | | |
|---|---|---|
| THF/H₂O (a_{w} = 0.6) | FIG. 12B | (RT-150° C.) |
| | | FIG. 12B |

aw = water activity
SE = slow evaporation
RT = room temperature

In some embodiments, a solid form described herein is a solid form of a freebase of Compound A.

In some embodiments, a solid form described herein is a hydrate and/or a solvate.

In some embodiments, a solid form described herein is anhydrous.

In some embodiments, a solid form of Compound A described herein is substantially free of any other solid form of Compound A (e.g., substantially free of any other crystalline and/or amorphous forms of Compound A). In some embodiments, a particular solid form of Compound A described herein comprises less than about 20%, 15%, 10%, 5%, or 1% by weight of any other solid form of Compound A. In some embodiments, a particular solid form of Compound A described herein comprises less than about 20% by weight of any other solid form of Compound A. In other embodiments, a particular solid form of Compound A described herein comprises less than about 15% by weight of any other solid form of Compound A. In still other embodiments, a particular solid form of Compound A described herein comprises less than about 10% by weight of any other solid form of Compound A. In yet still other embodiments, a particular solid form of Compound A described herein comprises less than about 5% by weight of any other solid form of Compound A. In some embodiments, a particular solid form of Compound A described herein comprises less than about 1% by weight of any other solid form of Compound A.

### Amorphous Form of Compound A

In some embodiments, a solid form of Compound A is an amorphous form of Compound A. In some embodiments, an amorphous form of Compound A can be used to prepare other solid form of Compound A (e.g., any of Solid Forms 1-12 as described herein).

In some embodiments, an amorphous Compound A is substantially free of any other solid form of Compound A. In some embodiments, an amorphous Compound A is substantially free of any crystalline form of Compound A. In some embodiments, an amorphous Compound A comprises less than about 20%, 15%, 10%, 5%, or 1% by weight of any other solid form of Compound A (e.g., any crystalline form of Compound A). In some embodiments, amorphous Compound A comprises less than about 20% by weight of any other solid form of Compound A (e.g., any crystalline form of Compound A). In other embodiments, amorphous Compound A comprises less than about 15% by weight of any other solid form of Compound A (e.g., any crystalline form of Compound A). In still other embodiments, amorphous Compound A comprises less than about 10% by weight of any other solid form of Compound A (e.g., any crystalline form of Compound A). In yet still other embodiments, amorphous Compound A comprises less than about 5% by weight of any other solid form of Compound A (e.g., any crystalline form of Compound A). In some embodiments, amorphous Compound A comprises less than about 1% by weight of any other solid form of Compound A (e.g., any crystalline form of Compound A).

### 1. Methods of Preparation

In some embodiments, an amorphous form of Compound A is prepared as described herein, including according to Table 2 and the Examples provided herein.

In some embodiments, ratios of solvents used in methods described herein are understood to be provided as volume:volume (v/v) ratios. For example, a ratio of x:y described herein can be interchangeable with the term x:y (v/v).

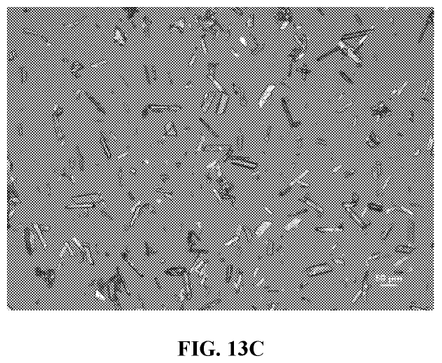

In some embodiments, an amorphous Compound A is prepared from freebase Solid Form E of Compound A (e.g., as prepared herein). A representative XRPD pattern of Form E of Compound A is depicted in FIG. 13A. Representative DSC and TGA thermograms of Form E of Compound A are depicted in FIG. 13B. A representative PLM image of Form E of Compound A is depicted in FIG. 13C.

In some embodiments, an amorphous Compound A is prepared by a process of mixing with THF at about 40° C., mixing under vacuum followed by drying at about 40° C. In some embodiments, wherein said process of the previous sentence is repeated, and the solid of amorphous form of Compound A is subject to further oven drying under vacuum at about 50° C. and sweep with dry nitrogen for about 3 days.

### 2. Characterization

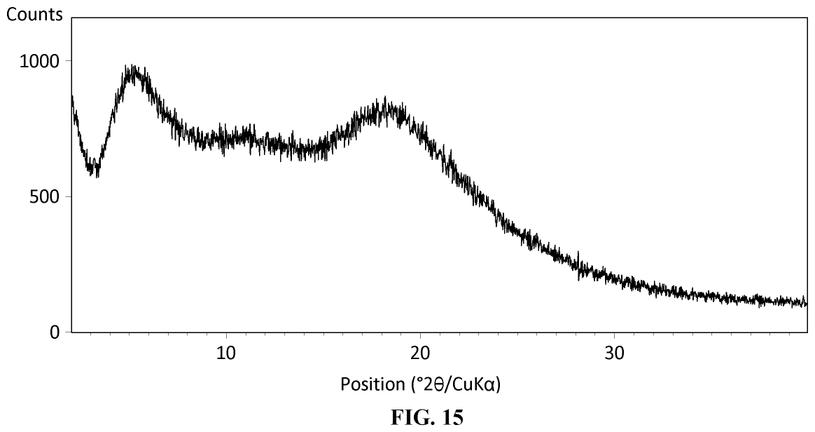

A representative XRPD pattern of an amorphous form of Compound A is depicted in FIG. 15.

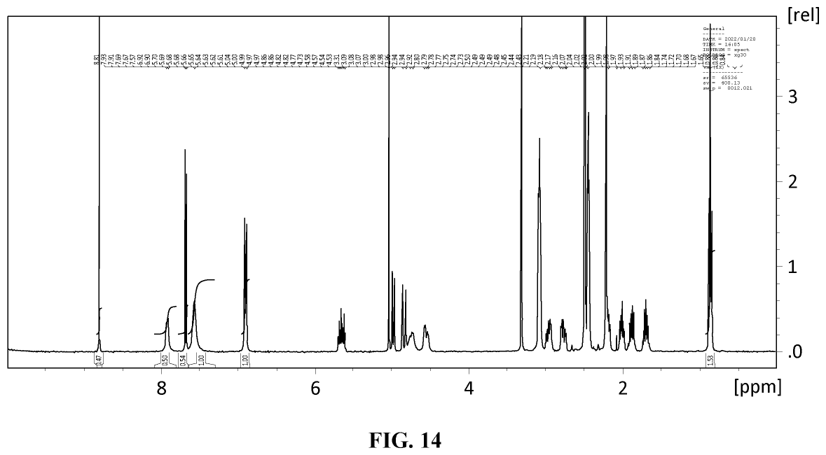

In some embodiments, ¹H NMR is used to determine the residual solvent in the amorphous form of Compound A. A representative ¹H NMR spectrum in DMSO-d₆ of Compound A is depicted in FIG. 14.

### Solid Form 1 of Compound A

In some embodiments, a solid form of Compound A is Solid Form 1.

### 1. Methods of Preparation

In some embodiments, Solid Form 1 of Compound A is obtained from a precursor form of Compound A. In some embodiments, a precursor form of Compound A is prepared according to a method comprising one or more steps as described in the examples provided herein. In some embodiments, a precursor form of Compound A is an amorphous form of Compound A.

In some embodiments, Solid Form 1 of Compound A can be prepared as described herein in Table 2 as well as in Example 2-1.

In one aspect, provided herein is a method of preparing Solid Form 1 of Compound A, said method comprising using a slurry of amorphous Compound A, wherein said slurry comprises the solvents of n-heptane and n-BuOH, and optionally wherein said slurry is heated to about 50° C. In some embodiments, the ratio of n-heptane and n-BuOH (n-heptane:n-BuOH) is about 8:2 in the preparation of Solid Form 1 of Compound A. In some embodiments, Solid Form 1 of Compound A can be prepared using a slurry at about 50° C. In some embodiments, Solid Form 1 of Compound A is prepared using a slurry of Compound A (e.g., amorphous Compound A) at about 50° C. using n-heptane:n-BuOH (8:2).

### 2. Characterization

In some embodiments, Solid Form 1 is characterized by any of the XRPD, DSC, and/or TGA properties described in Table 2.

A representative XRPD pattern of Solid Form 1 of Compound A is depicted in FIG. 1A. In some embodiments, Solid Form 1 of Compound A is characterized by an XRPD pattern substantially similar to one in FIG. 1A.

In some embodiments, Solid Form 1 of Compound A has an X-ray powder diffraction pattern comprising one, two, three, four, or five peaks at about 4.91° 2θ±0.2° 2θ, about 5.98° 2θ±0.2° 2θ, about 9.77° 2θ±0.2° 2θ, about 10.64° 2θ±0.2° 2θ, or about 17.40° 2θ±0.2° 2θ. In some embodiments, Solid Form 1 of Compound A is characterized by one or more peaks as described in the table below.

| Peak No. | Position at 2θ0 | Height (counts) |
|---|---|---|
| 1 | 4.16 | 348.39 |
| 2 | 4.91 | 11084.00 |
| 3 | 5.98 | 716.75 |
| 4 | 6.04 | 555.91 |
| 5 | 6.31 | 89.39 |
| 6 | 8.35 | 370.93 |
| 7 | 9.77 | 1178.52 |
| 8 | 10.64 | 809.62 |
| 9 | 11.92 | 146.61 |
| 10 | 12.23 | 437.46 |
| 11 | 12.42 | 178.64 |
| 12 | 13.20 | 198.20 |
| 13 | 14.28 | 89.72 |
| 14 | 15.48 | 302.49 |
| 15 | 15.68 | 148.10 |
| 16 | 16.17 | 64.95 |
| 17 | 16.72 | 164.63 |
| 18 | 17.16 | 271.45 |
| 19 | 17.40 | 1524.16 |
| 20 | 17.57 | 202.37 |
| 21 | 17.93 | 707.74 |
| 22 | 18.19 | 419.23 |
| 23 | 18.92 | 252.18 |
| 24 | 19.29 | 147.40 |
| 25 | 19.68 | 151.49 |
| 26 | 20.27 | 138.48 |
| 27 | 21.67 | 116.18 |
| 28 | 22.03 | 94.40 |
| 29 | 22.64 | 62.58 |
| 30 | 23.60 | 44.71 |
| 31 | 24.69 | 52.02 |
| 32 | 25.16 | 32.26 |
| 33 | 25.54 | 63.97 |
| 34 | 25.95 | 23.57 |
| 35 | 27.19 | 32.81 |
| 36 | 31.11 | 23.01 |

A representative DSC thermogram of Solid Form 1 of Compound A is depicted in FIG. 1B. In some embodiments, Solid Form 1 of Compound A is characterized by a DSC thermogram substantially similar to FIG. 1B.

In some embodiments, Solid Form 1 of Compound A has DSC endotherm peak at about 126° C., about 149° C., and/or about 168° C.

In some embodiments, Solid Form 1 of Compound A is characterized by a first DSC endotherm peak at about 126° C., a second DSC endotherm peak at about 149° C., and a third DSC endotherm peak at about 168° C.

A representative TGA thermogram of Solid Form 1 of Compound A is depicted in FIG. 1B. In some embodiments, Solid Form 1 of Compound A is characterized by a TGA thermogram substantially similar to FIG. 1B. In some embodiments, Solid Form 1 of Compound A has a TGA thermogram wherein said Solid Form 1 has a weight loss of about 2.78 wt. % when heated from about room temperature to about 150° C.

In some embodiments, Solid Form 1 of Compound A is substantially free of 2-12.

### Solid Form 2 of Compound A

In some embodiments, a solid form of Compound A is Solid Form 2.

### 1. Methods of Preparation

In some embodiments, Solid Form 2 of Compound A is prepared as described herein, including according to Table 2 and the Examples provided herein. In some embodiments, a precursor form of Compound A is prepared according to a method comprising one or more steps as described in the examples provided herein. In some embodiments, a precursor form of Compound A is an amorphous form of Compound A.

In some embodiments, Solid Form 2 of Compound A can be prepared as described herein in Table 2 as well as in Example 2-2.

In one aspect, provided herein is a method of preparing Solid Form 2 of Compound A, said method comprising anti-solvent addition, reverse anti-solvent addition, solid vapor diffusion, liquid vapor diffusion, a slurry at room temperature, a slurry at 50° C., slow evaporation, fast evaporation, slow cooling and/or temperature cycling. In some embodiments, Solid Form 2 of Compound A can be prepared using anti-solvent addition. In some embodiments, anti-solvent addition comprises the use of any one of the solvent combinations 1-10 of Table A-1.

TABLE A-1
Anti-Solvent addition for
Preparation of Solid Form 2

| Combination | Solvent 1 | Solvent 2 |
|---|---|---|
| 1 | Acetone | MTBE |
| 2 | MeCN | MTBE |
| 3 | Pyridine | MTBE |
| 4 | DCM | n-heptane |
| 5 | DCM | MTBE |
| 6 | Dioxane | Toluene |
| 7 | n-PrOH | MTBE |
| 8 | EtOAc | MTBE |
| 9 | IPAc | MTBE |
| 10 | 2-butanone | MTBE |

In some embodiments, Solid Form 2 of Compound A can be prepared using reverse anti-solvent addition. In some embodiments, reverse anti-solvent addition comprises the use of any one of the solvent combinations 1~4 of Table A-2.

TABLE A-2
Reverse Anti-Solvent addition for
Preparation of Solid Form 2

| Combination | Solvent 1 | Solvent 2 |
|---|---|---|
| 1 | DMF | MTBE |
| 2 | Diisopropyl ether | n-heptane |
| 3 | Diisopropyl ether | MTBE |
| 4 | Xylene | MTBE |

In some embodiments, Solid Form 2 of Compound A can be prepared using solid vapor diffusion. In some embodiments, solvent vapor diffusion comprises the use of diisopropyl ether, MIBK (methyl isobutyl ketone), MeOH, and/or IPAc (isopropyl acetate).

In some embodiments, Solid Form 2 of Compound A can be prepared using liquid vapor diffusion. In some embodiments, liquid vapor diffusion comprises the use of any one of the solvent combinations 1-6 of Table A-3.

TABLE A-3
Liquid vapor diffusion for
Preparation of Solid Form 2

| Combination | Solvent 1 | Solvent 2 |
|---|---|---|
| 1 | DMF | MTBE |
| 2 | n-BuOH | MTBE |
| 3 | EtOH | MTBE |
| 4 | EtOH | Toluene |
| 5 | CHCl₃ | IPAc |
| 6 | CHCl₃ | Toluene |

In some embodiments, said slurry for the preparation of Solid Form 2 comprises a solvent that is EtOAc and is optionally at room temperature. In some embodiments, Solid Form 2 of Compound A can be prepared using a slurry at room temperature. In some embodiments, a slurry comprises a solvent that is EtOAc (ethyl acetate).

In some embodiments, said slurry for the preparation of Solid Form 2 comprises a solvent system that is n-heptane/xylene, n-heptane/2-MeTHF, or n-heptane/MIBK, and optionally wherein said slurry is at 50° C. and/or the solvents are in a ratio of about 8:2 (e.g., n-heptane:xylene, n-heptane:2-MeTHF, or n-heptane:MIBK). In some embodiments, Solid Form 2 of Compound A can be prepared using a slurry at about 50° C. In some embodiments, a slurry comprises a solvent that is n-heptane. In some embodiments, a slurry comprises a solvent system that is n-heptane:xylene (8:2). In some embodiments, a slurry comprises a solvent system that is n-heptane:2-MeTHF (8:2). In some embodiments, a slurry comprises a solvent system that is n-heptane:MIBK (8:2).

In some embodiments, Solid Form 2 of Compound A can be prepared using slow evaporation at about 50° C. (e.g., wherein the slow evaporation uses a solvent that is MIBK) . . .

In some embodiments, Solid Form 2 of Compound A can be prepared using fast evaporation (e.g., wherein the fast evaporation uses a solvent that is toluene).

In some embodiments, Solid Form 2 of Compound A can be prepared using slow cooling. (for example, wherein the slow cooling uses a solvent that is diisopropylether)

In some embodiments, Solid Form 2 of Compound A can be prepared using temperature cycling. In some embodiments, temperature cycling comprises use of a solvent that is EtOAc or a combination of n-heptane/xylene (e.g., 2:1, n-heptane:xylene).

### 2. Characterization

In some embodiments, Solid Form 2 is characterized by any of the XRPD, DSC, and/or TGA properties described in Table 2.

A representative XRPD pattern of Solid Form 2 of Compound A is depicted in FIG. 2A. In some embodiments, Solid Form 2 of Compound A is characterized by an XRPD pattern substantially similar to one in FIG. 2A.

In some embodiments, Solid Form 2 of Compound A has an X-ray powder diffraction pattern comprising one, two, three, four, or five peaks at about 4.22° 2θ±0.2° 2θ, about 4.99° 2θ±0.2° 2θ, about 5.74° 2θ±0.2° 2θ, about 10.17° 2θ±0.2° 2θ, or about 16.04° 2θ±0.2° 2θ. In some embodiments, Solid Form 2 of Compound A is characterized by one or more peaks as described in the table below.

| Peak | Position | Height |
|---|---|---|
| No. | at 2θ | (counts) |
| 1 | 3.46 | 567.85 |
| 2 | 4.22 | 3611.10 |
| 3 | 4.99 | 3492.82 |
| 4 | 5.37 | 1113.84 |
| 5 | 5.74 | 1165.11 |
| 6 | 8.29 | 248.36 |
| 7 | 8.77 | 171.83 |
| 8 | 10.17 | 1845.26 |
| 9 | 10.87 | 494.94 |
| 10 | 11.73 | 398.87 |
| 11 | 12.29 | 69.10 |
| 12 | 13.17 | 237.77 |
| 13 | 14.73 | 289.13 |
| 14 | 16.04 | 1842.92 |
| 15 | 16.75 | 449.92 |
| 16 | 16.92 | 199.68 |
| 17 | 18.02 | 174.83 |
| 18 | 19.13 | 36.39 |
| 19 | 20.38 | 233.42 |
| 20 | 21.45 | 89.92 |
| 21 | 23.18 | 42.45 |
| 22 | 24.61 | 39.34 |
| 23 | 26.18 | 24.92 |
| 24 | 26.93 | 169.46 |

A representative DSC thermogram of Solid Form 2 of Compound A is depicted in FIG. 2B. In some embodiments, Solid Form 2 of Compound A is characterized by a DSC thermogram substantially similar to FIG. 2B.

In some embodiments, Solid Form 2 has DSC endotherm peak at about 134° C. and/or about 169° C., and/or has DSC exotherm peak at about 153° C.

In some embodiments, Solid Form 2 has a DSC thermogram, wherein a first DSC endotherm peak at about 134° C., a second DSC endotherm peak at about 169° C., and a first DSC exotherm peak at about 153° C.

A representative TGA thermogram of Form 2 of Compound A is depicted in FIG. 2B. In some embodiments, Solid Form 2 of Compound A is characterized by a TGA thermogram substantially similar to FIG. 2B. In some embodiments, Solid Form 2 has a TGA thermogram, wherein said Solid Form 2 has a weight loss of about 1.20 wt. % when heated from about room temperature to about 150° C.

In some embodiments, Solid Form 2 of Compound A is substantially free of any other solid form of Compound A, such as those described herein and including Solid Forms 1 and 3-12.

### Solid Form 3 of Compound A

In some embodiments, a solid form of Compound A is Solid Form 3.

### 1. Methods of Preparation

In some embodiments, Solid Form 3 of Compound A is obtained from a precursor form of Compound A. In some embodiments, a precursor form of Compound A is prepared according to a method comprising one or more steps as described in the examples provided herein. In some embodiments, a precursor form of Compound A is an amorphous form of Compound A.

In some embodiments, Solid Form 3 of Compound A can be prepared as described herein in Table 2 as well as in Example 2-3.

In one aspect, provided herein is a method of preparing Solid Form 3 of Compound A, said method comprising using an anti-solvent addition, liquid vapor diffusion, or a slurry at about 50° C. In some embodiments, Solid Form 3 of Compound A can be prepared using anti-solvent addition. In some embodiments, anti-solvent addition comprises the use of a solvent that is MTBE. In some embodiments, anti-solvent addition comprises the use of a solvent combination selected from: n-BuOH/MTBE, dioxane/MTBE, and IPA/MTBE.

In some embodiments, Solid Form 3 of Compound A can be prepared using liquid vapor diffusion (e.g., wherein the liquid vapor diffusion uses a solvent system comprising CHCl₃ and cyclohexane).

In some embodiments, Solid Form 3 of Compound A can be prepared using a slurry at about 50° C. (e.g., wherein the slurry is created using a solvent that is cyclohexane).

### 2. Characterization

In some embodiments, Solid Form 3 is characterized by any of the XRPD, DSC, and/or TGA properties described in Table 2.

A representative XRPD pattern of Solid Form 3 of Compound A is depicted in FIG. 3A. In some embodiments, Solid Form 3 of Compound A is characterized by an XRPD pattern substantially similar to one in FIG. 3A.

In some embodiments, Solid Form 3 of Compound A has an X-ray powder diffraction pattern comprising one, two, three, four, or five peaks at about 3.42° 2θ±0.2° 2θ, about 4.51° 2θ±0.2° 2θ, about 5.87° 2θ±0.2° 2θ, about 10.10° 2θ±0.2° 2θ, or about 16.67° 2θ±0.2° 2θ. In some embodiments, Solid Form 3 of Compound A is characterized by one or more peaks as described in the table below.

| Peak | Position | Height |
|---|---|---|
| No. | at 2θ | (counts) |
| 1 | 3.42 | 1278.37 |
| 2 | 4.51 | 2313.41 |
| 3 | 5.87 | 717.10 |
| 4 | 10.10 | 596.54 |
| 5 | 12.07 | 196.44 |
| 6 | 13.06 | 103.99 |
| 7 | 15.32 | 230.19 |
| 8 | 16.67 | 836.91 |
| 9 | 17.59 | 252.09 |
| 10 | 19.24 | 133.85 |
| 11 | 20.35 | 136.98 |
| 12 | 26.89 | 56.36 |

A representative DSC thermogram of Solid Form 3 of Compound A is depicted in FIG. 3B. In some embodiments, Solid Form 3 of Compound A is characterized by a DSC thermogram substantially similar to FIG. 3B.

In some embodiments, Solid Form 3 of Compound A has DSC endotherm peak at about 119° C., about 140° C. and/or about 167° C., and/or has DSC exotherm peak at about 156° C.

In some embodiments, Solid Form 3 of Compound A has a first DSC endotherm peak at about 119° C., a second DSC endotherm peak at about 140° C., a third DSC endotherm peak at about 167° C., and a first DSC exotherm peak at about 156° C.

A representative TGA thermogram of Form 3 of Compound A is depicted in FIG. 3B. In some embodiments, Solid Form 3 of Compound A is characterized by a TGA thermogram substantially similar to FIG. 3. In some embodiments, Solid Form 3 has a TGA thermogram, wherein said Solid Form 3 has a weight loss of about 2.26 wt. % when heated from about room temperature to about 150° C.

In some embodiments, Solid Form 3 of Compound A is substantially free of any other solid form of Compound A, such as those described herein and including Solid Forms 1, 2 and 4-12.

### Solid Form 4 of Compound A

In some embodiments, a solid form of Compound A is Solid Form 4.

### 1. Methods of Preparation

In some embodiments, Solid Form 4 of Compound A is obtained from a precursor form of Compound A. In some embodiments, a precursor form of Compound A is prepared according to a method comprising one or more steps as described in the examples provided herein. In some embodiments, a precursor form of Compound A is an amorphous form of Compound A.

In some embodiments, Solid Form 4 of Compound A can be prepared as described herein in Table 2 as well as in Example 2-4.

In some embodiments, Solid Form 4 of Compound A can be prepared using a slurry at room temperature. In some embodiments, a slurry for the preparation of Solid Form 4 comprises a solvent system of heptane and CH₂Cl₂, and optionally wherein said solvents are in a ratio of about 8:2 (heptane:CH₂Cl₂).

### 2. Characterization

In some embodiments, Solid Form 4 is characterized by any of the XRPD properties described in Table 2.

A representative XRPD pattern of Solid Form 4 of Compound A is depicted in FIG. 4. In some embodiments, Solid Form 4 of Compound A is characterized by an XRPD pattern substantially similar to one in FIG. 4.

In some embodiments, Solid Form 4 of Compound A has an X-ray powder diffraction pattern comprising one, two, three, four, or five peaks at about 4.16° 2θ±0.2° 2θ, about 5.18° 2θ±0.2° 2θ, about 5.61° 2θ±0.2° 2θ, about 10.39° 2θ±0.2° 2θ, or about 16.65° 2θ±0.2° 2θ. In some embodiments, Solid Form 4 of Compound A is characterized by one or more peaks as described in the table below.

| Peak | Position | Height |
|---|---|---|
| No. | at 2θ | (counts) |
| 1 | 3.37 | 162.38 |
| 2 | 4.16 | 2111.49 |
| 3 | 4.35 | 588.11 |
| 4 | 5.18 | 5571.86 |
| 5 | 5.61 | 1030.92 |
| 6 | 6.42 | 127.06 |
| 7 | 8.49 | 128.60 |
| 8 | 9.43 | 288.29 |
| 9 | 9.84 | 137.36 |
| 10 | 10.13 | 506.05 |
| 11 | 10.39 | 901.49 |
| 12 | 11.20 | 141.30 |
| 13 | 11.68 | 91.68 |
| 14 | 12.61 | 101.96 |
| 15 | 13.88 | 37.72 |
| 16 | 14.68 | 88.38 |
| 17 | 15.07 | 114.28 |
| 18 | 15.67 | 113.92 |
| 19 | 16.00 | 201.55 |
| 20 | 16.65 | 653.47 |
| 21 | 18.13 | 125.95 |
| 22 | 19.09 | 91.18 |
| 23 | 20.87 | 27.99 |
| 24 | 21.38 | 74.73 |
| 25 | 21.97 | 29.13 |
| 26 | 26.02 | 30.86 |

In some embodiments, Solid Form 4 of Compound A is substantially free of 1-3 and 5-12.

### Solid Form 5 of Compound A

In some embodiments, a solid form of Compound A is Solid Form 5.

### 1. Methods of Preparation

In some embodiments, Solid Form 5 of Compound A is obtained from a precursor form of Compound A. In some embodiments, a precursor form of Compound A is prepared according to a method comprising one or more steps as described in the examples provided herein. In some embodiments, a precursor form of Compound A is an amorphous form of Compound A.

In some embodiments, Solid Form 5 of Compound A can be prepared as described herein in Table 2 as well as in Example 2-5.

In one aspect, provided herein is a method of preparing Solid Form 5 of Compound A, said method comprising using anti-solvent addition, reverse anti-solvent addition, or a slurry at about room temperature. In some embodiments, Solid Form 5 of Compound A can be prepared using anti-solvent addition. In some embodiments, anti-solvent addition comprises the use of a solvent that is MTBE. In some embodiments anti-solvent addition comprises the use of a solvent system selected from: benzene/MTBE, MeOH/MTBE, and THF/MTBE.

In some embodiments, Solid Form 5 of Compound A can be prepared using reverse anti-solvent addition (e.g., wherein the reverse anti-solvent addition uses pyridine/toluene).

In some embodiments, Solid Form 5 of Compound A can be prepared using a slurry at room temperature. In some embodiments, a slurry for the preparation of Solid Form 5 comprises a solvent system of heptane and dioxane, and optionally wherein said solvents are in a ratio of about 8:2 (heptane:dioxane).

### 2. Characterization

In some embodiments, Solid Form 5 is characterized by any of the XRPD, DSC, and/or TGA properties described in Table 2.

A representative XRPD pattern of Solid Form 5 of Compound A is depicted in FIG. 5A. In some embodiments, Solid Form 5 of Compound A is characterized by an XRPD pattern substantially similar to one in FIG. 5A.

In some embodiments, Solid Form 5 of Compound A has an X-ray powder diffraction pattern comprising one, two, three, four, or five peaks at about 3.40° 2θ±0.2° 2θ, about 4.48° 2θ±0.2° 2θ, about 5.18° 2θ±0.2° 2θ, about 5.86° 2θ±0.2° 2θ, or about 10.30° 2θ±0.2° 2θ. In some embodiments, Solid Form 5 of Compound A is characterized by one or more peaks as described in the table below.

| Peak | Position | Height |
|---|---|---|
| No. | at 2θ | (counts) |
| 1 | 3.40 | 1085.08 |
| 2 | 4.48 | 2774.53 |
| 3 | 5.18 | 1377.04 |
| 4 | 5.86 | 513.12 |
| 5 | 7.60 | 124.99 |
| 6 | 8.94 | 95.19 |
| 7 | 10.30 | 503.71 |
| 8 | 12.20 | 112.44 |
| 9 | 13.49 | 61.78 |
| 10 | 14.44 | 128.51 |
| 11 | 15.53 | 228.70 |
| 12 | 16.52 | 405.45 |
| 13 | 17.00 | 202.07 |
| 14 | 18.06 | 123.94 |
| 15 | 19.14 | 138.59 |
| 16 | 20.53 | 102.65 |
| 17 | 21.36 | 103.46 |
| 18 | 23.37 | 59.50 |
| 19 | 27.28 | 23.39 |

A representative DSC thermogram of Solid Form 5 of Compound A is depicted in FIG. 5B. In some embodiments, Solid Form 5 of Compound A is characterized by a DSC thermogram substantially similar to FIG. 5B.

A representative TGA thermogram of Solid Form 5 of Compound A is depicted in FIG. 5B. In some embodiments, Solid Form 5 of Compound A is characterized by a TGA thermogram substantially similar to FIG. 5B. In some embodiments, Solid Form 5 of Compound A has a TGA thermogram, wherein said Solid Form 5 has a weight loss of about 9.08 wt. % when heated from about room temperature to about 150° C.

In some embodiments, Solid Form 5 of Compound A is substantially free of any other solid form of Compound A, such as those described herein and including Solid Forms 1-4 and 6-12.

### Solid Form 6 of Compound A

In some embodiments, a solid form of Compound A is Solid Form 6.

### 1. Methods of Preparation

In some embodiments, Solid Form 6 of Compound A is obtained from a precursor form of Compound A. In some embodiments, a precursor form of Compound A is prepared according to a method comprising one or more steps as described in the examples provided herein. In some embodiments, a precursor form of Compound A is an amorphous form of Compound A.

In some embodiments, Solid Form 6 of Compound A can be prepared as described herein in Table 2 as well as in Example 2-6.

In one aspect, provided herein is a method of preparing Solid Form 6 of Compound A, said method comprising using reverse anti-solvent addition or liquid vapor diffusion. In some embodiments, Solid Form 6 of Compound A can be prepared using reverse anti-solvent addition. In some embodiments, reverse anti-solvent addition comprises the use of acetic acid. In some embodiments, reverse anti-solvent addition comprises the use of a solvent system that is acetic acid/MTBE or acetic acid/toluene.

In some embodiments, Solid Form 6 of Compound A can be prepared using liquid vapor diffusion. In some embodiments, liquid vapor diffusion comprises the use of a solvent system that is acetic acid/MTBE.

### 2. Characterization

In some embodiments, Solid Form 6 is characterized by any of the XRPD, DSC, and/or TGA properties described in Table 2.

A representative XRPD pattern of Solid Form 6 of Compound A is depicted in FIG. 6A. In some embodiments, Solid Form 6 of Compound A is characterized by an XRPD pattern substantially similar to one in FIG. 6A.

In some embodiments, Solid Form 6 of Compound A has an X-ray powder diffraction pattern comprising one, two, three, four, or five peaks at about 5.25° 2θ±0.2° 2θ, about 11.26° 2θ±0.2° 2θ, about 12.70° 2θ±0.2° 2θ, about 15.34° 2θ±0.2° 2θ, or about 16.71° 2θ±0.2° 2θ. In some embodiments, Solid Form 6 of Compound A is characterized by one or more peaks as described in the table below.

| Peak | Position | Height |
|---|---|---|
| No. | at 2θ | (counts) |
| 1 | 5.25 | 385.56 |
| 2 | 9.73 | 125.48 |
| 3 | 11.26 | 351.83 |
| 4 | 11.81 | 22.01 |
| 5 | 12.70 | 234.32 |
| 6 | 14.88 | 100.74 |
| 7 | 15.34 | 255.72 |
| 8 | 15.91 | 59.02 |
| 9 | 16.71 | 340.51 |
| 10 | 18.31 | 44.14 |
| 11 | 18.65 | 77.78 |
| 12 | 19.50 | 142.51 |
| 13 | 19.87 | 60.99 |
| 14 | 21.82 | 20.49 |
| 15 | 22.12 | 82.02 |
| 16 | 22.80 | 56.88 |
| 17 | 23.17 | 68.5 |
| 18 | 23.59 | 162.91 |
| 19 | 25.51 | 22.41 |
| 20 | 26.48 | 16.09 |
| 21 | 27.28 | 26.79 |
| 22 | 32.69 | 13.02 |

A representative DSC thermogram of Solid Form 6 of Compound A is depicted in FIG. 6B. In some embodiments, Solid Form 6 of Compound A is characterized by a DSC thermogram substantially similar to FIG. 6B. In some embodiments, Solid Form 6 has DSC endotherm peak at about 142° C.

A representative TGA thermogram of Solid Form 6 of Compound A is depicted in FIG. 6B. In some embodiments, Solid Form 6 of Compound A is characterized by a TGA thermogram substantially similar to FIG. 6B. In some embodiments, Solid Form 6 of Compound A has a TGA thermogram, wherein said Solid Form 6 has a weight loss of about 0.75 wt. % when heated from about room temperature to about 100° C.

In some embodiments, Solid Form 6 of Compound A is substantially free of any other solid form of Compound A, such as those described herein and including Solid Forms 1-5 and 7-12.

### Solid Form 7 of Compound A

In some embodiments, a solid form of Compound A is Solid Form 7.

### 1. Methods of Preparation

In some embodiments, Solid Form 7 of Compound A is obtained from a precursor form of Compound A. In some embodiments, a precursor form of Compound A is prepared according to a method comprising one or more steps as described in the examples provided herein. In some embodiments, a precursor form of Compound A is an amorphous form of Compound A.

In some embodiments, Solid Form 7 of Compound A can be prepared as described herein in Table 2 as well as in Example 2-7.

In one aspect, provided herein is a method of preparing Solid Form 7 of Compound A, said method comprising using reverse anti-solvent addition, liquid vapor diffusion or polymer induced crystallization. In some embodiments, Solid Form 7 of Compound A can be prepared using reverse anti-solvent addition. In some embodiments, reverse anti-solvent addition comprises the use of a solvent system selected from: benzene/toluene, n-BuOH/n-heptane, and n-PrOH/n-heptane.

In some embodiments, Solid Form 7 of Compound A can be prepared using liquid vapor diffusion (e.g., wherein the liquid vapor diffusion uses a solvent system comprising n-BuOH/n-heptane).

In some embodiments, Solid Form 7 of Compound A can be prepared using polymer induced crystallization. In some embodiments, polymer induced crystallization for the preparation of Solid Form 7 comprises the use of a polymer that is hydroxypropyl methylcellulose (HPMC) and/or a solvent system that is MTBE/IPA, and optionally in a ratio of about 1:1 (MTBE:IPA).

### 2. Characterization

In some embodiments, Solid Form 7 is characterized by any of the XRPD, DSC, and/or TGA properties described in Table 2.

A representative XRPD pattern of Solid Form 7 of Compound A is depicted in FIG. 7A. In some embodiments, Solid Form 7 of Compound A is characterized by an XRPD pattern substantially similar to one in FIG. 7A.

In some embodiments, Solid Form 7 of Compound A has an X-ray powder diffraction pattern comprising one, two, three, four, or five peaks at about 5.16° 2θ±0.2° 2θ, about 9.34° 2θ±0.2° 2θ, about 16.40° 2θ±0.2° 2θ, about 16.52° 2θ±0.2° 2θ, or about 22.44° 2θ±0.2° 2θ. In some embodiments, Solid Form 7 of Compound A is characterized by one or more peaks as described in the table below.

| Peak | Position | Height |
|---|---|---|
| No. | at 2θ | (counts) |
| 1 | 5.16 | 10335.87 |
| 2 | 7.57 | 34.94 |
| 3 | 9.34 | 188.04 |
| 4 | 10.33 | 174.56 |
| 5 | 11.26 | 133.76 |
| 6 | 12.15 | 100.28 |
| 7 | 13.23 | 27.31 |
| 8 | 14.03 | 38.1 |
| 9 | 14.70 | 40.63 |
| 10 | 15.09 | 132.57 |
| 11 | 15.44 | 180.92 |
| 12 | 16.22 | 78.83 |
| 13 | 16.40 | 286.02 |
| 14 | 16.52 | 271.47 |
| 15 | 17.17 | 66.65 |
| 16 | 18.65 | 147.85 |
| 17 | 18.81 | 96.34 |
| 18 | 20.75 | 59.9 |
| 19 | 21.81 | 99.07 |
| 20 | 22.44 | 184.32 |
| 21 | 24.44 | 41.68 |
| 22 | 26.56 | 38.63 |
| 23 | 30.93 | 27.5 |

A representative DSC thermogram of Solid Form 7 of Compound A is depicted in FIG. 7B. In some embodiments, Solid Form 7 of Compound A is characterized by a DSC thermogram substantially similar to FIG. 7B.

In some embodiments, Solid Form 7 of Compound A has DSC endotherm peak at about 80° C. and/or about 169° C., and/or has DSC exotherm peak at about 143° C. In some embodiments, a first DSC endotherm peak at about 80° C., a second DSC endotherm peak at about 169° C., and a first DSC exotherm peak at about 143° C.

A representative TGA thermogram of Solid Form 7 of Compound A is depicted in FIG. 7B. In some embodiments, Solid Form 7 of Compound A is characterized by a TGA thermogram substantially similar to FIG. 7B. In some embodiments, Solid Form 7 has a TGA thermogram, wherein said Solid Form 7 has a weight loss of about 18.1 wt. % when heated from about room temperature to about 150° C.

In some embodiments, Solid Form 7 of Compound A is substantially free of any other solid form of Compound A, such as those described herein and including Solid Forms 1-6 and 8-12.

### Solid Form 8 of Compound A

In some embodiments, a solid form of Compound A is Solid Form 8.

### 1. Methods of Preparation

In some embodiments, Solid Form 8 of Compound A is obtained from a precursor form of Compound A. In some embodiments, a precursor form of Compound A is prepared according to a method comprising one or more steps as described in the examples provided herein. In some embodiments, a precursor form of Compound A is an amorphous form of Compound A.

In some embodiments, Solid Form 8 of Compound A can be prepared as described herein in Table 2 as well as in Example 2-8.

In some embodiments, Solid Form 8 of Compound A can be prepared using anti-solvent addition. In some embodiments, anti-solvent addition comprises a solvent system that is DMAc/n-heptane.

In some embodiments, Solid Form 8 of Compound A can be prepared using a slurry at room temperature. In some embodiments, a slurry comprises use of a solvent system that is MTBE:DMF (optionally at 6:4).

### 2. Characterization

In some embodiments, Solid Form 8 is characterized by any of the XRPD, DSC, and/or TGA properties described in Table 2.

A representative XRPD pattern of Solid Form 8 of Compound A is depicted in FIG. 8A. In some embodiments, Solid Form 8 of Compound A is characterized by an XRPD pattern substantially similar to one in FIG. 8A.

In some embodiments, Solid Form 8 of Compound A has an X-ray powder diffraction pattern comprising one, two, three, four, or five peaks at about 5.02° 2θ±0.2° 2θ, about 11.81° 2θ±0.2° 2θ, about 15.03° 2θ±0.2° 2θ, about 16.41° 2θ±0.2° 2θ, or about 20.09° 2θ±0.2° 2θ. In some embodiments, Solid Form 8 of Compound A is characterized by one or more peaks as described in the table below.

| Peak | Position | Height |
|---|---|---|
| No. | at 2θ | (counts) |
| 1 | 5.02 | 2840.14 |
| 2 | 6.22 | 25.49 |
| 3 | 7.66 | 129.92 |
| 4 | 9.45 | 108.23 |
| 5 | 10.03 | 259 |
| 6 | 11.25 | 52.18 |
| 7 | 11.45 | 121.13 |
| 8 | 11.81 | 156.02 |
| 9 | 12.21 | 66.77 |
| 10 | 13.76 | 94.52 |
| 11 | 14.34 | 53.51 |
| 12 | 15.03 | 443.85 |
| 13 | 15.31 | 70.91 |
| 14 | 16.16 | 207 |
| 15 | 16.41 | 157.51 |
| 16 | 18.91 | 108.38 |
| 17 | 19.37 | 24.8 |
| 18 | 19.75 | 91.61 |
| 19 | 20.09 | 181.77 |
| 20 | 22.42 | 72.54 |
| 21 | 23.01 | 27.89 |
| 22 | 23.31 | 61.4 |
| 23 | 24.55 | 25.31 |
| 24 | 25.31 | 32.81 |

A representative DSC thermogram of Solid Form 8 of Compound A is depicted in FIG. 8B. In some embodiments, Solid Form 8 of Compound A is characterized by a DSC thermogram substantially similar to FIG. 8B. In some embodiments, Solid Form 8 of Compound A has DSC endotherm peak at about 75° C. and/or about 99° C. In some embodiments, Solid Form 8 of Compound A has a first DSC endotherm peak at about 75° C., and a second DSC endotherm peak at about 99° C.

A representative TGA thermogram of Solid Form 8 of Compound A is depicted in FIG. 8B. In some embodiments, Solid Form 8 of Compound A is characterized by a TGA thermogram substantially similar to FIG. 8B. In some embodiments, Solid Form 8 has a TGA thermogram, wherein said Solid Form 8 has a weight loss of about 6.0 wt. % when heated from about room temperature to about 125° C., and/or about 9.55 wt. % when heated from about 125° C. to about 252° C.

In some embodiments, Solid Form 8 of Compound A is substantially free of 1-7 and 9-12.

### Solid Form 9 of Compound A

In some embodiments, a solid form of Compound A is Solid Form 9.

### 1. Methods of Preparation

In some embodiments, Solid Form 9 of Compound A is obtained from a precursor form of Compound A. In some embodiments, a precursor form of Compound A is prepared according to a method comprising one or more steps as described in the examples provided herein. In some embodiments, a precursor form of Compound A is an amorphous form of Compound A.

In some embodiments, Solid Form 9 of Compound A can be prepared as described herein in Table 2 as well as in Example 2-9.

In some embodiments, Solid Form 9 of Compound A can be prepared using reverse anti-solvent addition (e.g., using a solvent system comprising xylene and n-heptane).

### 2. Characterization

In some embodiments, Solid Form 9 is characterized by any of the XRPD, DSC, and/or TGA properties described in Table 2.

A representative XRPD pattern of Solid Form 9 of Compound A is depicted in FIG. 9A. In some embodiments, Solid Form 9 of Compound A is characterized by an XRPD pattern substantially similar to one in FIG. 9A.

In some embodiments, Solid Form 9 of Compound A has an X-ray powder diffraction pattern comprising one, two, three, four, or five peaks at about 4.20° 2θ±0.2° 2θ, about 4.47° 2θ±0.2° 2θ, about 5.20° 2θ±0.2° 2θ, about 6.43° 2θ±0.2° 2θ, or about 10.46° 2θ±0.2° 2θ. In some embodiments, Solid Form 9 of Compound A is characterized by one or more peaks as described in the table below.

| Peak | Position | Height |
|---|---|---|
| No. | at 2θ | (counts) |
| 1 | 4.20 | 549.92 |
| 2 | 4.47 | 816.52 |
| 3 | 5.00 | 321.49 |
| 4 | 5.20 | 1064.71 |
| 5 | 6.43 | 558.92 |
| 6 | 8.92 | 45.96 |
| 7 | 10.46 | 574.85 |
| 8 | 11.42 | 88.56 |
| 9 | 11.95 | 146.88 |
| 10 | 12.62 | 212.19 |
| 11 | 13.43 | 50.5 |
| 12 | 13.88 | 50.37 |
| 13 | 14.99 | 384.6 |
| 14 | 16.58 | 89.92 |
| 15 | 18.15 | 260.29 |
| 16 | 18.95 | 45.54 |
| 17 | 19.71 | 108.25 |
| 18 | 21.29 | 145.77 |
| 19 | 21.95 | 28.5 |
| 20 | 24.71 | 22.9 |
| 21 | 27.17 | 15.61 |
| 22 | 29.42 | 17.25 |

A representative DSC thermogram of Solid Form 9 of Compound A is depicted in FIG. 9B. In some embodiments, Solid Form 9 of Compound A is characterized by a DSC thermogram substantially similar to FIG. 9B.

In some embodiments, Solid Form 9 of Compound A has DSC endotherm peak at about 130° C., and/or about 168° C., and/or has DSC exotherm peak at about 143° C. In some embodiments, Solid Form 9 of Compound A has a first DSC endotherm peak at about 130° C., a second DSC endotherm peak at about 168° C., and a first DSC exotherm peak at about 143° C.

A representative TGA thermogram of Solid Form 9 of Compound A is depicted in FIG. 9B. In some embodiments, Solid Form 9 of Compound A is characterized by a TGA thermogram substantially similar to FIG. 9B. In some embodiments, Solid Form 9 of Compound A has a TGA thermogram, wherein said Solid Form 9 has a weight loss of about 0.99 wt. % when heated from about room temperature to about 150° C.

In some embodiments, Solid Form 9 of Compound A is substantially free of 1-8 and 10-12.

### Solid Form 10 of Compound A

In some embodiments, a solid form of Compound A is Solid Form 10.

### 1. Methods of Preparation

In some embodiments, Solid Form 10 of Compound A is obtained from a precursor form of Compound A. In some embodiments, a precursor form of Compound A is prepared according to a method comprising one or more steps as described in the examples provided herein. In some embodiments, a precursor form of Compound A is an amorphous form of Compound A.

In some embodiments, Solid Form 10 of Compound A can be prepared as described herein in Table 2 as well as in Example 2-10.

In some embodiments, Solid Form 10 of Compound A can be prepared using slow cooling (e.g., wherein the slow cooling comprises the use of a solvent that is CHCl₃).

### 2. Characterization

In some embodiments, Solid Form 10 is characterized by any of the XRPD properties described in Table 2.

A representative XRPD pattern of Solid Form 10 of Compound A is depicted in FIG. 10. In some embodiments, Solid Form 10 of Compound A is characterized by an XRPD pattern substantially similar to one in FIG. 10.

In some embodiments, Solid Form 10 of Compound A has an X-ray powder diffraction pattern comprising one, two, three, four, or five peaks at about 4.96° 2θ±0.2° 2θ, about 5.13° 2θ±0.2° 2θ, about 5.53° 2θ±0.2° 2θ, about 10.22° 2θ±0.2° 2θ, or about 11.15° 2θ±0.2° 2θ. In some embodiments, Solid Form 10 of Compound A is characterized by one or more peaks as described in the table below.

In some embodiments, Solid Form 10 of Compound A, wherein said Solid Form is substantially free of any other solid form of Compound A, such as those described herein and including Solid Forms 1-9, 11 and 12.

| Peak | Position | Height |
|---|---|---|
| No. | at 2θ | (counts) |
| 1 | 4.96 | 1337.21 |
| 2 | 5.13 | 4346.08 |
| 3 | 5.53 | 724.18 |
| 4 | 9.12 | 117.37 |
| 5 | 10.22 | 937.43 |
| 6 | 11.15 | 194.81 |
| 7 | 11.38 | 180.63 |
| 8 | 12.7 | 31.44 |
| 9 | 14.22 | 28.57 |
| 10 | 15.36 | 97.98 |
| 11 | 16.16 | 135.97 |
| 12 | 18.06 | 106.86 |
| 13 | 20.50 | 96.45 |

### Solid Form 11 of Compound A

In some embodiments, a solid form of Compound A is Solid Form 11.

### 1. Methods of Preparation

In some embodiments, Solid Form 11 of Compound A is obtained from a precursor form of Compound A. In some embodiments, a precursor form of Compound A is prepared according to a method comprising one or more steps as described in the examples provided herein. In some embodiments, a precursor form of Compound A is an amorphous form of Compound A.

In some embodiments, Solid Form 11 of Compound A can be prepared as described herein in Table 2 as well as in Example 2-11.

In some embodiments, Solid Form 11 of Compound A can be prepared using sonication induced nucleation (e.g., wherein the sonication comprises the use of a solvent that is EtOAc).

### 2. Characterization

In some embodiments, Solid Form 11 is characterized by any of the XRPD, DSC, and/or TGA properties described in Table 2.

A representative XRPD pattern of Solid Form 11 of Compound A is depicted in FIG. 11A. In some embodiments, Solid Form 11 of Compound A is characterized by an XRPD pattern substantially similar to one in FIG. 11A.

In some embodiments, Solid Form 11 of Compound A has an X-ray powder diffraction pattern comprising one, two, three, four, or five peaks at about 4.38° 2θ±0.2° 2θ, about 5.20° 2θ±0.2° 2θ, about 6.43° 2θ±0.2° 2θ, about 10.45° 2θ±0.2° 2θ, or about 12.61° 2θ±0.2° 2θ. In some embodiments, Solid Form 11 of Compound A is characterized by one or more peaks as described in the table below.

| Peak | Position | Height |
|---|---|---|
| No. | at 2θ | (counts) |
| 1 | 4.38 | 570.77 |
| 2 | 5.20 | 1450.02 |
| 3 | 6.43 | 253.03 |
| 4 | 8.99 | 52.58 |
| 5 | 10.45 | 356.29 |
| 6 | 11.45 | 93.17 |
| 7 | 11.96 | 58.12 |
| 8 | 12.61 | 196.86 |
| 9 | 13.41 | 35.23 |
| 10 | 13.86 | 41.36 |
| 11 | 14.99 | 195.31 |
| 12 | 16.61 | 191.5 |
| 13 | 18.14 | 108.13 |
| 14 | 19.02 | 41.05 |
| 15 | 19.32 | 33.59 |
| 16 | 19.77 | 54.88 |
| 17 | 21.36 | 65.43 |
| 18 | 21.98 | 62.09 |
| 19 | 22.83 | 22.12 |
| 20 | 24.78 | 77.34 |
| 21 | 25.88 | 53.26 |
| 22 | 27.21 | 18.65 |

A representative DSC thermogram of Solid Form 11 of Compound A is depicted in FIG. 11B. In some embodiments, Solid Form 11 of Compound A is characterized by a DSC thermogram substantially similar to FIG. 11B.

In some embodiments, Solid Form 11 of Compound A has DSC endotherm peak at about 134° C., and/or about 168° C.

In some embodiments, Solid Form 11 has a first DSC endotherm peak at about 134° C., and a second DSC endotherm peak at about 168° C.

A representative TGA thermogram of Solid Form 11 of Compound A is depicted in FIG. 11B. In some embodiments, Solid Form 11 of Compound A is characterized by a TGA thermogram substantially similar to FIG. 11B. In some embodiments, Solid Form 11 has a TGA thermogram, wherein said Solid Form 11 has a weight loss of about 1.13 wt. % when heated from about room temperature to about 100° C.

In some embodiments, Solid Form 11 of Compound A is substantially free of any other solid form of Compound A, such as those described herein and including Solid Forms 1-10 and 12.

### Solid Form 12 of Compound A

In some embodiments, a solid form of Compound A is Solid Form 12.

### 1. Methods of Preparation

In some embodiments, Solid Form 12 of Compound A is obtained from a precursor form of Compound A. In some embodiments, a precursor form of Compound A is prepared according to a method comprising one or more steps as described in the examples provided herein. In some embodiments, a precursor form of Compound A is an amorphous form of Compound A.

In some embodiments, Solid Form 12 of Compound A can be prepared as described herein in Table 2 as well as in Example 2-12.

In some embodiments, Solid Form 12 of Compound A can be prepared using a slurry at room temperature. In some embodiments, a slurry comprises use of a solvent system of THF/H₂O. In some embodiments, the THF/H₂O has a water activity (a_{w}) of about 0.2, about 0.4, about 0.6, or about 0.8.

### 2. Characterization

In some embodiments, Solid Form 12 is characterized by any of the XRPD, DSC, and/or TGA properties described in Table 2.

A representative XRPD pattern of Solid Form 12 of Compound A is depicted in FIG. 12A. In some embodiments, Solid Form 12 of Compound A is characterized by an XRPD pattern substantially similar to one in FIG. 12A.

In some embodiments, Solid Form 12 of Compound A has an X-ray powder diffraction pattern comprising one, two, three, four, or five peaks at about 5.23° 2θ±0.2° 2θ, about 15.64° 2θ±0.2° 2θ, about 16.48° 2θ±0.2° 2θ, about 16.67° 2θ±0.2° 2θ, or about 21.34° 2θ±0.2° 2θ. In some embodiments, Solid Form 12 of Compound A is characterized by one or more peaks as described in the table below.

| Peak | Position | Height |
|---|---|---|
| No. | at 2θ | (counts) |
| 1 | 5.23 | 12002.78 |
| 2 | 7.58 | 710.57 |
| 3 | 8.89 | 134.17 |
| 4 | 9.59 | 444.51 |
| 5 | 10.45 | 493.07 |
| 6 | 11.01 | 776.63 |
| 7 | 12.57 | 660.21 |
| 8 | 13.02 | 83.64 |
| 9 | 13.60 | 77.99 |
| 10 | 14.61 | 605.84 |
| 11 | 15.64 | 1094.85 |
| 12 | 16.48 | 2372.80 |
| 13 | 16.67 | 2883.84 |
| 14 | 18.27 | 446.85 |
| 15 | 19.05 | 449.21 |
| 16 | 19.52 | 657.89 |
| 17 | 19.80 | 353.36 |
| 18 | 20.59 | 697.62 |
| 19 | 20.93 | 462.49 |
| 20 | 21.34 | 922.87 |
| 21 | 21.63 | 692.16 |
| 22 | 22.63 | 256.98 |
| 23 | 23.00 | 246.19 |
| 24 | 23.35 | 714.85 |
| 25 | 23.69 | 477.15 |
| 26 | 24.81 | 222.43 |
| 27 | 25.43 | 294.74 |
| 28 | 26.83 | 245.82 |
| 29 | 29.15 | 63.21 |
| 30 | 30.33 | 105.31 |
| 31 | 34.76 | 36.33 |

A representative DSC thermogram of Solid Form 12 of Compound A is depicted in FIG. 12B. In some embodiments, Solid Form 12 of Compound A is characterized by a DSC thermogram substantially similar to FIG. 12B.

In some embodiments, Solid Form 12 has DSC endotherm peak at about 90° C.

A representative TGA thermogram of Solid Form 12 of Compound A is depicted in FIG. 12B. In some embodiments, Solid Form 12 of Compound A is characterized by a TGA thermogram substantially similar to FIG. 12B. In some embodiments, Solid Form 2 has a TGA thermogram, wherein said Solid Form 12 has a weight loss of about 8.66 wt. % when heated from about room temperature to about 150° C.

In some embodiments, Solid Form 12 of Compound A is substantially free of any other solid form of Compound A, such as those described herein and including Solid Forms 1-11.

### V. Uses and Methods of Treatment

Solid forms of Compound A (including any of Solid Forms 1-12 described herein) can be useful in therapy. Accordingly, solid forms of Compound A (including any of Solid Forms 1-12 described herein) can be used for treating or preventing various diseases and conditions in a subject in need thereof as well as used in the manufacture of a medicament for treating or preventing various diseases and conditions in a subject in need thereof. Exemplary methods and uses are described herein.

Some embodiments described herein relate to a method for ameliorating and/or treating a cancer that can include administering an effective amount of a solid form of Compound A described herein (e.g., any of Solid Forms 1-12 of Compound A), or a pharmaceutical composition that includes a solid form of Compound A described herein (e.g., any of Solid Forms 1-12 of Compound A), to a subject having a cancer described herein. Other embodiments described herein relate to the use of an effective amount of a solid form of Compound A described herein (e.g., any of Solid Forms 1-12 of Compound A), or a pharmaceutical composition that includes a solid form of Compound A described herein (e.g., any of Solid Forms 1-12 of Compound A), in the manufacture of a medicament for treating a cancer described herein. Still other embodiments described herein relate to an effective amount of a solid form of Compound A described herein (e.g., any of Solid Forms 1-12 of Compound A), or a pharmaceutical composition that includes a solid form of Compound A described herein (e.g., any of Solid Forms 1-12 of Compound A), for treating a cancer described herein.

Some embodiments described herein is a method for ameliorating or treating a malignant growth or tumor in a subject in need thereof, said method comprising administering to the subject an effective amount of a solid form of Compound A described herein (e.g., any one of Solid Forms 1-12), or any pharmaceutical composition thereof. Other embodiments described herein relate to the use of an effective amount of a solid form of Compound A described herein (e.g., any of Solid Forms 1-12 of Compound A), or a pharmaceutical composition that includes a solid form of Compound A described herein (e.g., any of Solid Forms 1-12 of Compound A), in the manufacture of a medicament for treating a malignant growth or tumor described herein, for example, a malignant growth or tumor from a cancer described herein. Still other embodiments described herein relate to an effective amount of a solid form of Compound A described herein (e.g., any of Solid Forms 1-12 of Compound A), or a pharmaceutical composition that includes a solid form of Compound A described herein (e.g., any of Solid Forms 1-12 of Compound A), for treating a malignant growth or tumor described herein, for example, a malignant growth or tumor from a cancer described herein.

Some embodiments described herein relate to a method for ameliorating or treating a cancer described herein that can include contacting a cancer cell with an effective amount of a solid form of Compound A described herein (e.g., any of Solid Forms 1-12 of Compound A), or a pharmaceutical composition that includes a solid form of Compound A described herein (e.g., any of Solid Forms 1-12 of Compound A) to a subject having a cancer described herein. Other embodiments described herein relate to the use of an effective amount of a solid form of Compound A described herein (e.g., any of Solid Forms 1-12 of Compound A), or a pharmaceutical composition that includes a solid form of Compound A described herein (e.g., any of Solid Forms 1-12 of Compound A) in the manufacture of a medicament for ameliorating or treating a cancer that can include contacting a cancer cell. Still other embodiments described herein relate to an effective amount of a solid form of Compound A described herein (e.g., any of Solid Forms 1-12 of Compound A), or a pharmaceutical composition that includes a solid form of Compound A described herein (e.g., any of Solid Forms 1-12 of Compound A) for ameliorating or treating a cancer that can include contacting a cancer cell.

Some embodiments described herein is a method for ameliorating or treating a malignant growth or tumor in a subject in need thereof, said method comprising contacting the malignant grown or tumor with an effective amount of a solid form of Compound A described herein (e.g., any one of Solid Forms 1-12), or any pharmaceutical composition thereof. Other embodiments described herein relate to the use of an effective amount of a solid form of Compound A described herein (e.g., any of Solid Forms 1-12 of Compound A), or a pharmaceutical composition that includes a solid form of Compound A described herein (e.g., any of Solid Forms 1-12 of Compound A) in the manufacture of a medicament for ameliorating or treating a malignant growth or tumor, for example, a malignant growth or tumor from a cancer described herein. Still other embodiments described herein relate to an effective amount of a solid form of Compound A described herein (e.g., any of Solid Forms 1-12 of Compound A), or a pharmaceutical composition that includes a solid form of Compound A described herein (e.g., any of Solid Forms 1-12 of Compound A) for ameliorating or treating a malignant growth or tumor, for example, a malignant growth or tumor from a cancer described herein.

Some embodiments described herein relate to a method for inhibiting the activity of WEE1 (for example, inhibiting the activity of WEE1 in TP53-mutated cells, inhibiting the activity of WEE1 in TP53 wild-type cells, inhibiting the activity in WEE1 p53-deficient cells and/or decreasing the overexpression of WEE1 in cells) that can include providing an effective amount of a solid form of Compound A described herein (e.g., any of Solid Forms 1-12 of Compound A), or a pharmaceutical composition that includes a solid form of Compound A described herein (e.g., any of Solid Forms 1-12 of Compound A) to a cancer cell from a cancer described herein. Other embodiments described herein relate to the use of an effective amount of a solid form of Compound A described herein (e.g., any of Solid Forms 1-12 of Compound A) or a pharmaceutical composition that includes an effective amount of a solid form of Compound A described herein (e.g., any of Solid Forms 1-12 of Compound A) in the manufacture of a medicament for inhibiting the activity of WEE1 (for example, inhibiting the activity of WEE1 in TP53-mutated cells, inhibiting the activity of WEE1 in TP53 wild-type cells, inhibiting the activity in WEE1 p53-deficient cells and/or decreasing the overexpression of WEE1 in cells). Still other embodiments described herein relate to an effective amount of a solid form of Compound A described herein (e.g., any of Solid Forms 1-12 of Compound A) or a pharmaceutical composition that includes an effective amount of a solid form of Compound A described herein (e.g., any of Solid Forms 1-12 of Compound A) for inhibiting the activity of WEE1 (for example, inhibiting the activity of WEE1 in TP53-mutated cells, inhibiting the activity of WEE1 in TP53 wild-type cells, inhibiting the activity in WEE1 p53-deficient cells and/or decreasing the overexpression of WEE1 in cells). Some embodiments described herein relate to a method for inhibiting the activity of WEE1 (for example, inhibiting the activity of WEE1 in TP53-mutated cells, inhibiting the activity of WEE1 in TP53 wild-type cells, inhibiting the activity in WEE1 p53-deficient cells and/or decreasing the overexpression of WEE1 in cells) that can include providing an effective amount of a solid form of Compound A described herein (e.g., any of Solid Forms 1-12 of Compound A) or a pharmaceutical composition that includes an effective amount of a solid form of Compound A described herein (e.g., any of Solid Forms 1-12 of Compound A) to a cancer cell from a cancer described herein. Other embodiments described herein relate to a method for inhibiting the activity of WEE1 (for example, inhibiting the activity of WEE1 in TP53-mutated cells, inhibiting the activity of WEE1 in TP53 wild-type cells, inhibiting the activity in WEE1 p53-deficient cells and/or decreasing the overexpression of WEE1 in cells) that can include contacting a cancer cell from a cancer described herein with an effective amount of a solid form of Compound A described herein (e.g., any of Solid Forms 1-12 of Compound A) or a pharmaceutical composition that includes an effective amount of a solid form of Compound A described herein (e.g., any of Solid Forms 1-12 of Compound A), and thereby inhibiting the activity of WEE1.

Some embodiments described herein relate to a method for ameliorating or treating a cancer described herein that can include inhibiting the activity of WEE1 (for example, inhibiting the activity of WEE1 in TP53-mutated cells, inhibiting the activity of WEE1 in TP53 wild-type cells, inhibiting the activity in WEE1 p53-deficient cells and/or decreasing the overexpression of WEE1 in cells) using an effective amount of a solid form of Compound A described herein (e.g., any of Solid Forms 1-12 of Compound A), or a pharmaceutical composition that includes a solid form of Compound A described herein (e.g., any of Solid Forms 1-12 of Compound A). Other embodiments described herein relate to the use of an effective amount of a solid form of Compound A described herein (e.g., any of Solid Forms 1-12 of Compound A), or a pharmaceutical composition that includes a solid form of Compound A described herein (e.g., any of Solid Forms 1-12 of Compound A) in the manufacture of a medicament for ameliorating or treating a cancer described herein by inhibiting the activity of WEE1 (for example, inhibiting the activity of WEE1 in TP53-mutated cells, inhibiting the activity of WEE1 in TP53 wild-type cells, inhibiting the activity in WEE1 p53-deficient cells and/or decreasing the overexpression of WEE1 in cells). Still other embodiments described herein relate to an effective amount of a solid form of Compound A described herein (e.g., any of Solid Forms 1-12 of Compound A), or a pharmaceutical composition that includes a solid form of Compound A described herein (e.g., any of Solid Forms 1-12 of Compound A) for ameliorating or treating a cancer described herein by inhibiting the activity of WEE1 (for example, inhibiting the activity of WEE1 in TP53-mutated cells, inhibiting the activity of WEE1 in TP53 wild-type cells, inhibiting the activity in WEE1 p53-deficient cells and/or decreasing the overexpression of WEE1 in cells). Some embodiments described herein relate to a method for ameliorating or treating a cancer described herein that can include contacting a cancer cell with an effective amount of a solid form of Compound A described herein (e.g., any of Solid Forms 1-12 of Compound A), or a pharmaceutical composition that includes a solid form of Compound A described herein (e.g., any of Solid Forms 1-12 of Compound A) wherein the Solid Form of Compound A inhibits the activity of WEE1 (for example, inhibiting the activity of WEE1 in TP53-mutated cells, inhibiting the activity of WEE1 in TP53 wild-type cells, inhibiting the activity in WEE1 p53-deficient cells and/or decreasing the overexpression of WEE1 in cells).

Some embodiments described herein relate to a method for inhibiting replication of a malignant growth or a tumor described herein that can include contacting the growth or the tumor with an effective amount of a solid form of Compound A described herein (e.g., any of Solid Forms 1-12 of Compound A), or a pharmaceutical composition that includes a solid form of Compound A described herein (e.g., any of Solid Forms 1-12 of Compound A). Other embodiments described herein relate to the use of an effective amount of a solid form of Compound A described herein (e.g., any of Solid Forms 1-12 of Compound A), or a pharmaceutical composition that includes a solid form of Compound A described herein (e.g., any of Solid Forms 1-12 of Compound A), in the manufacture of a medicament for inhibiting replication of a malignant growth or a tumor described herein. In some embodiments, the use can include contacting the growth or the tumor with the medicament. Still other embodiments described herein relate to an effective amount of a solid form of Compound A described herein (e.g., any of Solid Forms 1-12 of Compound A), or a pharmaceutical composition that includes a solid form of Compound A described herein (e.g., any of Solid Forms 1-12 of Compound A), for inhibiting replication of a malignant growth or a tumor described herein.

Examples of suitable cancers include, but are not limited to: brain cancers, cervicocerebral cancers, esophageal cancers, thyroid cancers, small cell cancers, non-small cell cancers, breast cancers, lung cancers (for example non-small cell lung cancer and small cell lung cancer), stomach cancers, gallbladder/bile duct cancers, liver cancers, pancreatic cancers, colon cancers, rectal cancers, ovarian cancers, choriocarcinomas, cervical cancers, uterine cancers, vaginal cancers, vulvar cancers, uterus body cancers, uterocervical cancers, renal pelvis/ureter cancers, bladder cancers, prostate cancers, penis cancers, testicular cancers, fetal cancers, Wilms' cancer, skin cancers, malignant melanoma, neuroblastomas, osteosarcomas, Ewing's tumors, soft part sarcomas, acute leukemia, chronic lymphatic leukemias, chronic myelocytic leukemias, polycythemia vera, malignant lymphomas, multiple myeloma, Hodgkin's lymphomas, and non-Hodgkin's lymphomas. In some embodiments, a cancer is a breast cancer, a cervical cancer, an ovarian cancer, a uterine cancer, a vaginal cancer, or a vulvar cancer.

In some embodiments, an ovarian cancer is a platinum sensitive ovarian cancer. In some embodiments, an ovarian cancer is a cyclin E1 driven ovarian cancer. In some embodiments, an ovarian cancer is PARP resistant ovarian cancer.

In some embodiments, a cancer is uterine serous carcinoma.

In some embodiments, a cancer is a solid tumor.

In some embodiments, a cancer is osteosarcoma.

In some embodiments, a cancer is colorectal cancer (e.g., BRAF mutant colorectal cancer).

In some embodiments, a cancer is pancreatic cancer.

As described herein, a cancer can become resistant to one or more anti-cancer agents. In some embodiments, a solid form of Compound A described herein (e.g., any of Solid Forms 1-12 of Compound A), or a pharmaceutical composition that includes an effective amount of a solid form of Compound A described herein (e.g., any of Solid Forms 1-12 of Compound A), can be used to treat and/or ameliorate a cancer that has become resistant to one or more anti-cancer agents (such as one or more WEE1 inhibitors). Examples of anti-cancer agents that a subject may have developed resistance to include, but are not limited to, WEE1 inhibitors (such as AZD1775 or adavosertib). In some embodiments, the cancer that has become resistant to one or more anti-cancer agents can be a cancer described herein.

Several known WEE1 inhibitors can cause one or more undesirable side effects in the subject being treated. Examples of undesirable side effects include, but are not limited to, thrombocytopenia, neutropenia, anemia, diarrhea, vomiting, nausea, abdominal pain, and constipation. In some embodiments, a solid form of Compound A described herein (e.g., any of Solid Forms 1-12 of Compound A) can decrease the number and/or severity of one or more side effects associated with a known WEE1 inhibitor. In some embodiments, a solid form of Compound A described herein (e.g., any of Solid Forms 1-12 of Compound A), can result in a severity of a side effect (such as one of those described herein) that is 25% less than compared to the severity of the same side effect experienced by a subject receiving a known WEE1 inhibitor (such as AZD1775, formally known as MK1775 (CAS No.: 955365-80-7, 2-allyl-1-(6-(2-hydroxypropan-2-yl)pyridin-2-yl)-6-(4-(4-methylpiperazin-1-yl)phenylamino)-1,2-dihydropyrazolo[3,4-d]pyrimidin-3-one)). In some embodiments, a solid form of Compound A described herein (e.g., any of Solid Forms 1-12 of Compound A), results in a number of side effects that is 25% less than compared to the number of side effects experienced by a subject receiving a known WEE1 inhibitor (for example, AZD1775). In some embodiments, a solid form of Compound A described herein (e.g., any of Solid Forms 1-12 of Compound A), results in a severity of a side effect (such as one of those described herein) that is less in the range of about 10% to about 30% compared to the severity of the same side effect experienced by a subject receiving a known WEE1 inhibitor (such as AZD1775) In some embodiments, a solid form of Compound A described herein (e.g., any of Solid Forms 1-12 of Compound A), results in a number of side effects that is in the range of about 10% to about 30% less than compared to the number of side effects experienced by a subject receiving a known WEE1 inhibitor (for example, AZD1775).

The one or more solid forms of Compound A as described herein, or a pharmaceutical composition that includes a solid form of Compound A described herein (e.g., any of Solid Forms 1-12 of Compound A), that can be used to treat, ameliorate and/or inhibit the replication of a cancer, malignant growth, or tumor wherein inhibiting the activity of WEE1 is beneficial is provided in any of the embodiments described herein. For example, in various embodiments, the methods and uses described above in the Uses and Methods of Treatment section of this disclosure are carried out in the described manner (generally involving cancer, malignant growth, and/or tumor) using a solid form of Compound A as described herein, or a pharmaceutical composition that includes a solid form of Compound A as described herein.

As used herein, a “subject” refers to an animal that is the object of treatment, observation or experiment. “Animal” includes cold- and warm-blooded vertebrates and invertebrates such as fish, shellfish, reptiles and, in particular, mammals. “Mammal” includes, without limitation, mice, rats, rabbits, guinea pigs, dogs, cats, sheep, goats, cows, horses, primates, such as monkeys, chimpanzees, and apes, and, in particular, humans. In some embodiments, the subject can be human. In some embodiments, the subject can be a child and/or an infant, for example, a child or infant with a fever. In other embodiments, the subject can be an adult.

As used herein, the terms “treat,” “treating,” “treatment,” “therapeutic,” and “therapy” do not necessarily mean total cure or abolition of the disease or condition. Any alleviation of any undesired signs or symptoms of the disease or condition, to any extent can be considered treatment and/or therapy. Furthermore, treatment may include acts that may worsen the subject's overall feeling of well-being or appearance.

The terms “therapeutically effective amount” and “effective amount” are used to indicate an amount of an active compound, or pharmaceutical agent, that elicits the biological or medicinal response indicated. For example, a therapeutically effective amount of a Solid Form of a compound, salt or composition can be the amount needed to prevent, alleviate or ameliorate symptoms of the disease or condition, or prolong the survival of the subject being treated. This response may occur in a tissue, system, animal or human and includes alleviation of the signs or symptoms of the disease or condition being treated. Determination of an effective amount is well within the capability of those skilled in the art, in view of the disclosure provided herein. The therapeutically effective amount of a solid form of Compound A as described herein, or a pharmaceutical composition that includes a solid form of Compound A described herein (e.g., any of Solid Forms 1-12 of Compound A), that is required as a dose will depend on the route of administration, the type of animal, including human, being treated and the physical characteristics of the specific animal under consideration. The dose can be tailored to achieve a desired effect, but will depend on such factors as weight, diet, concurrent medication and other factors which those skilled in the medical arts will recognize.

For example, an effective amount of a solid form of Compound A described herein (e.g., any of Solid Forms 1-12 of Compound A), or a pharmaceutical composition that includes a solid form of Compound A described herein (e.g., any of Solid Forms 1-12 of Compound A), is the amount that results in: (a) the reduction, alleviation or disappearance of one or more symptoms caused by the cancer, (b) the reduction of tumor size, (c) the elimination of the tumor, and/or (d) long-term disease stabilization (growth arrest) of the tumor. In the treatment of lung cancer (such as non-small cell lung cancer), a therapeutically effective amount is that amount that alleviates or eliminates cough, shortness of breath and/or pain.

As another example, an effective amount, or a therapeutically effective amount of an WEE1 inhibitor is the amount which results in the reduction in WEE1 activity and/or phosphorylation (such as phosphorylation of CDC2). The reduction in WEE1 activity is known to those skilled in the art and can be determined by the analysis of WEE1 intrinsic kinase activity and downstream substrate phosphorylation. As another example, an effective amount, or a therapeutically effective amount of a solid form of Compound A described herein (e.g., any of Solid Forms 1-12 of Compound A), or a pharmaceutical composition that includes a solid form of Compound A described herein (e.g., any of Solid Forms 1-12 of Compound A), is the amount which results in the reduction in WEE1 activity and/or phosphorylation (such as phosphorylation of CDC2). The reduction in WEE1 activity is known to those skilled in the art and can be determined by the analysis of WEE1 intrinsic kinase activity and downstream substrate phosphorylation.

The amount of a solid form of Compound A as described herein, or a pharmaceutical composition that includes a solid form of Compound A described herein (e.g., any of Solid Forms 1-12 of Compound A), required for use in treatment will vary not only with the particular compound or salt selected but also with the route of administration, the nature and/or symptoms of the disease or condition being treated and the age and condition of the subject and will be ultimately at the discretion of the attendant physician or clinician. In cases of administration of a pharmaceutically acceptable salt, dosages may be calculated as the free base. As will be understood by those of skill in the art, in certain situations it may be necessary to administer the compounds disclosed herein in amounts that exceed, or even far exceed, the dosage ranges described herein in order to effectively and aggressively treat particularly aggressive diseases or conditions.

In general, however, a suitable dose will often be in the range of from about 0.05 mg/kg to about 10 mg/kg. For example, a suitable dose may be in the range from about 0.10 mg/kg to about 7.5 mg/kg of body weight per day, such as about 0.15 mg/kg to about 5.0 mg/kg of body weight of the recipient per day, about 0.2 mg/kg to 4.0 mg/kg of body weight of the recipient per day, or any amount in between. The compound may be administered in unit dosage form; for example, containing 1 to 500 mg, 10 to 100 mg, 5 to 50 mg or any amount in between, of active ingredient per unit dosage form.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations.

As will be readily apparent to one skilled in the art, the useful in vivo dosage to be administered and the particular mode of administration will vary depending upon the age, weight, the severity of the affliction, the mammalian species treated, the particular compounds employed and the specific use for which these compounds are employed. The determination of effective dosage levels, that is the dosage levels necessary to achieve the desired result, can be accomplished by one skilled in the art using routine methods, for example, human clinical trials, in vivo studies and in vitro studies. For example, useful dosages of a solid form of Compound A described herein (e.g., any of Solid Forms 1-12 of Compound A), or a pharmaceutical composition that includes a solid form of Compound A described herein (e.g., any of Solid Forms 1-12 of Compound A), can be determined by comparing their in vitro activity and in vivo activity in animal models. Such comparison can be done by comparison against an established drug, such as cisplatin and/or gemcitabine).

Dosage amount and interval may be adjusted individually to provide plasma levels of the active moiety which are sufficient to maintain the modulating effects, or minimal effective concentration (MEC). The MEC will vary for each compound but can be estimated from in vivo and/or in vitro data. Dosages necessary to achieve the MEC will depend on individual characteristics and route of administration. However, HPLC assays or bioassays can be used to determine plasma concentrations. Dosage intervals can also be determined using MEC value. Compositions should be administered using a regimen which maintains plasma levels above the MEC for 10-90% of the time, preferably between 30-90% and most preferably between 50-90%. In cases of local administration or selective uptake, the effective local concentration of the drug may not be related to plasma concentration.

It should be noted that the attending physician would know how to and when to terminate, interrupt or adjust administration due to toxicity or organ dysfunctions. Conversely, the attending physician would also know to adjust treatment to higher levels if the clinical response were not adequate (precluding toxicity). The magnitude of an administrated dose in the management of the disorder of interest will vary with the severity of the disease or condition to be treated and to the route of administration. The severity of the disease or condition may, for example, be evaluated, in part, by standard prognostic evaluation methods. Further, the dose and perhaps dose frequency, will also vary according to the age, body weight and response of the individual subject. A program comparable to that discussed above may be used in veterinary medicine.

Solid forms of Compound A as described herein, or a pharmaceutical composition that includes a solid form of Compound A as described herein, can be evaluated for efficacy and toxicity using known methods. For example, the toxicology of a particular compound, or of a subset of the compounds, sharing certain chemical moieties, may be established by determining in vitro toxicity towards a cell line, such as a mammalian, and preferably human, cell line. The results of such studies are often predictive of toxicity in animals, such as mammals, or more specifically, humans. Alternatively, the toxicity of particular compounds in an animal model, such as mice, rats, rabbits, dogs or monkeys, may be determined using known methods. The efficacy of a particular compound may be established using several recognized methods, such as in vitro methods, animal models, or human clinical trials. When selecting a model to determine efficacy, the skilled artisan can be guided by the state of the art to choose an appropriate model, dose, route of administration and/or regime.

In some embodiments, methods and uses described herein comprises administration of a solid form of Compound A as described herein (e.g., any of Solid Forms 1-12, including as described herein) as monotherapy.

In some embodiments, a solid form of Compound A as described herein (e.g., any of Solid Forms 1-12, including as described herein) can be used as monotherapy for treatment of a cancer (e.g., uterine serous carcinoma, cyclin E1 drive high grade serous ovarian cancer, or a solid tumor), including those described herein.

In some embodiments, methods and uses described herein comprises administration of an effective amount of a solid form of Compound A as described herein (e.g., any of Solid Forms 1-12, including as described herein) in combination with one or more additional therapeutic agents (e.g., for the treatment of cancer). In some embodiments, a one or more additional therapeutic agent is a chemotherapy agent, a DNA damaging agent, or a molecularly targeted agent. In some embodiments, provided herein is a method of treating PARP resistant ovarian cancer comprising administration of an effective amount of a solid form of Compound A as described herein (e.g., any of Solid Forms 1-12, including as described herein) in combination with a PARP inhibitor. In some embodiments, provided herein is a method of treating ovarian cancer comprising administration of an effective amount of a solid form of Compound A as described herein (e.g., any of Solid Forms 1-12, including as described herein) in combination with chemotherapy. In some embodiments, provided herein is a method of treating osteosarcoma comprising administration of an effective amount of a solid form of Compound A as described herein (e.g., any of Solid Forms 1-12, including as described herein) in combination with gemcitabine. In some embodiments, provided herein a method of treating BRAF V600E-mutated colorectal cancer comprising administration of an effective amount of a solid form of Compound A as described herein (e.g., any of Solid Forms 1-12, including as described herein) in combination with encorafenib and cetuximab.

### VI. Pharmaceutical Compositions

Some embodiments described herein relate to a pharmaceutical composition, that can include an effective amount of a solid form of Compound A as described herein (e.g., any of Solid Forms 1-12, including as described herein), and a pharmaceutically acceptable carrier, diluent, excipient, or combination thereof.

The term “pharmaceutical composition” refers to a mixture of one or more compounds, such as compounds, salts and/or salt forms described herein, disclosed herein with other chemical components, such as diluents or carriers. The pharmaceutical composition facilitates administration of the compound, such as a solid form of Compound A described herein (e.g., any of Solid Forms 1-12 of Compound A), to an organism. Pharmaceutical compositions can also be obtained by reacting compounds with inorganic or organic acids such as hydrochloric acid, sulfuric acid, nitric acid, phosphoric acid, methanesulfonic acid, p-toluenesulfonic acid, adipic acid, maleic acid, tartaric acid, mucic acid, and hippuric acid. Pharmaceutical compositions will generally be tailored to the specific intended route of administration.

The term “physiologically acceptable” defines a carrier, diluent or excipient that does not abrogate the biological activity and properties of the compound, such as a compound, a salt and/or a salt form described herein, nor cause appreciable damage or injury to an animal to which delivery of the composition is intended.

As used herein, a “carrier” refers to a compound that facilitates the incorporation of a compound, such as a compound, a salt and/or a salt form described herein, into cells or tissues. For example, without limitation, dimethyl sulfoxide (DMSO) is a commonly utilized carrier that facilitates the uptake of many organic compounds into cells or tissues of a subject.

As used herein, a “diluent” refers to an ingredient in a pharmaceutical composition that lacks appreciable pharmacological activity but may be pharmaceutically necessary or desirable. For example, a diluent may be used to increase the bulk of a potent drug whose mass is too small for manufacture and/or administration. It may also be a liquid for the dissolution of a drug to be administered by injection, ingestion, or inhalation. A common form of diluent in the art is a buffered aqueous solution such as, without limitation, phosphate buffered saline that mimics the pH and isotonicity of human blood.

As used herein, an “excipient” refers to an essentially inert substance that is added to a pharmaceutical composition to provide, without limitation, bulk, consistency, stability, binding ability, lubrication, disintegrating ability etc., to the composition. For example, stabilizers such as anti-oxidants and metal-chelating agents are excipients. In an embodiment, the pharmaceutical composition comprises an anti-oxidant and/or a metal-chelating agent. A “diluent” is a type of excipient.

The pharmaceutical compositions described herein can be administered to a human subject per se, or in pharmaceutical compositions where they are mixed with other active ingredients, as in combination therapy, or carriers, diluents, excipients, or combinations thereof. Proper formulation is dependent upon the route of administration chosen. Techniques for formulation and administration of the compounds, salts, salt forms and/or compositions described herein are known to those skilled in the art.

The pharmaceutical compositions disclosed herein may be manufactured in a manner that is itself known, e.g., by means of conventional mixing, dissolving, granulating, dragee-making, levigating, emulsifying, encapsulating, entrapping or tableting processes. Additionally, the active ingredients are contained in an amount effective to achieve its intended purpose.

Multiple techniques of administering a compound, a salt, a salt form and/or a composition exist in the art including, but not limited to, oral, rectal, pulmonary, topical, aerosol, injection, infusion and parenteral delivery, including intramuscular, subcutaneous, intravenous, intramedullary injections, intrathecal, direct intraventricular, intraperitoneal, intranasal and intraocular injections.

One may also administer a compound, a salt, a salt form and/or a composition in a local rather than systemic manner, for example, via injection or implantation of a compound, a salt, a salt form and/or a composition directly into the affected area, often in a depot or sustained release formulation. Furthermore, one may administer a compound, a salt, a salt form and/or a composition in a targeted drug delivery system, for example, in a liposome coated with a tissue-specific antibody. The liposomes will be targeted to and taken up selectively by the organ. For example, intranasal or pulmonary delivery to target a respiratory disease or condition may be desirable.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Compositions that can include a compound, salt and/or salt form described herein formulated in a compatible pharmaceutical carrier may also be prepared, placed in an appropriate container, and labeled for treatment of an indicated condition.

### VII. Examples

Additional embodiments are disclosed in further detail in the following examples, which are not in any way intended to limit the scope of the claims.

Abbreviations Used

| | |
|---|---|
| BuOH | butanol |
| n-BuOH | N-butanol |
| DCM | dichloromethane |
| DMAc | dimethylacetamide |
| DMF | dimethylformamide |
| DSC | Differential scanning calorimetry |
| Endo | Endothermic peak |
| EtOAc | Ethyl acetate |
| Exo | Exothermic peak |
| HPMC | Hydroxypropyl methylcellulose |
| IPA | Isopropanol |
| IPAc | Isopropyl Acetate |
| IR | Infrared |
| MeOH | methanol |
| MIBK | Methyl isobutyl ketone |
| MTBE | Methyl tert-butyl ether |
| NMR | Nuclear magnetic resonance |
| PLM | Polarized light microscopy |
| n-PrOH | n-propanol |
| RT | Room temperature-approximately 25° C. |
| SE | Slow evaporation |
| TGA | Thermal gravimetric analysis |
| TGIR | Thermogravimetric Analysis coupled to |
| | Fourier Transform Infrared |
| THF | tetrahydrofuran |
| XRPD | X-ray powder diffraction |

Exemplary solid forms described herein can be obtained using an amorphous form of Compound A. In some embodiments, an amorphous form of Compound A is obtained from a crystalline freebase starting material, including freebase Form E as described herein as confirmed by XRPD and as characterized by polarized light microscopy (PLM), thermogravimetric analysis (TGA), differential scanning calorimetry (DSC), and nuclear magnetic resonance (1H-NMR). Based on the available data, the HPLC purity of the material was >99.99%. This starting material can then be used to prepare amorphous freebase using THE as solvent where the largest batch processed was ~10.0 g of crystalline material. The amorphous material can then be used to prepare the exemplary new Solid Forms described herein.

12 exemplary new Solid Forms referred to as Compounds 1 to 12 are described herein.

### Example 1. Amorphous Freebase Form of Compound A

The freebase amorphous form of Compound A was obtained from a precursor, freebase Form E of Compound A. Freebase Form E solid (10.0 g) was dissolved in THF (50.0 mL) at 40° C., rotavated at 40° C. under vacuum, followed by drying at 40° C. A process for obtaining Form E is provided in WO 2022/011391. The process was repeated and the solid of amorphous form of Compound A was subjected to further oven drying under vacuum at 50° C. and swept with dry nitrogen for 3 days.

A representative XRPD pattern of the precursor, freebase Form E of Compound A, is depicted in FIG. 13A. Representative DSC and TGA thermograms of freebase Form E of Compound A are depicted in FIG. 13B. A representative PLM image of freebase Form E of Compound A is depicted in FIG. 13C.

A representative XRPD pattern of an amorphous form of Compound A is depicted in FIG. 15. ¹H NMR is used to determine the residual solvent in the amorphous form of Compound A. A representative ¹H NMR is depicted in FIG. 14.

### Example 2-1. Solid Form 1 of Compound A

Solid Form 1 of Compound A was obtained via slurring amorphous freebase of Compound A in n-heptane/BuOH (8:2, v:v) at 50° C. About 30 mg of amorphous freebase of Compound A was suspended and stirred in n-heptane/BuOH (8:2 v:v) in a 4 mL vial at 50° C.

### Example 2-2. Solid Form 2 of Compound A

Solid Form 2 of Compound A was obtained via slurring amorphous freebase of Compound A in n-heptane/xylene (8:2, v:v) at 50° C. About 30 mg of amorphous freebase of Compound A was suspended and stirred in n-heptane/xylene (8:2, v:v) in a 4 mL vial at 50° C.

### Example 2-3. Solid Form 3 of Compound A

Solid Form 3 of Compound A was obtained via slurring amorphous freebase of Compound A in cyclohexane at 50° C. About 30 mg of amorphous freebase of Compound A was suspended and stirred in cyclohexane in a 4 mL vial at 50° C.

### Example 2-4. Solid Form 4 of Compound A

Solid Form 4 of Compound A was obtained via slurring amorphous freebase of Compound A in n-heptane/DCM (8:2, v:v) at room temperature. About 30 mg of amorphous freebase of Compound A was suspended and stirred in n-heptane/DCM (8:2, v:v) in a 4 mL vial at room temperature.

### Example 2-5. Solid Form 5 of Compound A

Solid Form 5 of Compound A was obtained via slurring amorphous freebase of Compound A in n-heptane/dioxane (8:2, v:v) at room temperature. About 30 mg of amorphous freebase of Compound A was suspended and stirred in n-heptane/dioxane (8:2, v:v) in a 4 mL vial at room temperature.

### Example 2-6. Solid Form 6 of Compound A

Solid Form 6 of Compound A was obtained from amorphous freebase of Compound A via reverse anti-solvent addition. About 30 mg of amorphous freebase of Compound A was dissolved in acetic acid to obtain a saturated solution which was then added MTBE dropwise.

### Example 2-7. Solid Form 7 of Compound A

Solid Form 7 of Compound A was obtained from amorphous freebase of Compound A via reverse anti-solvent addition. About 30 mg of amorphous freebase of Compound A was dissolved in n-BuOH to obtain a saturated solution which was then added n-heptane dropwise.

### Example 2-8. Solid Form 8 of Compound A

Solid Form 8 of Compound A was obtained from amorphous freebase of Compound A via reverse anti-solvent addition. About 30 mg of amorphous freebase of Compound A was dissolved in DMAc to obtain a saturated solution which was then added n-heptane dropwise.

### Example 2-9. Solid Form 9 of Compound A

Solid Form 9 of Compound A was obtained from amorphous freebase of Compound A via reverse anti-solvent addition. About 30 mg of amorphous freebase of Compound A was dissolved in xylene to obtain a saturated solution which was then added n-heptane dropwise.

### Example 2-10. Solid Form 10 of Compound A

Solid Form 10 of Compound A was obtained from amorphous freebase of Compound A via slow cooling. About 30 mg of amorphous freebase of Compound A was dissolved in chloroform (SE) to obtain saturated solutions in 4 mL vials at 55° C. The temperature was kept at 55° C. for 30 mins, decreased to 50° C. over 4 hours, then to 45° C. over 4 hours, then to 35° C. 4 hours, and finally to 25° C. over 4 hours, using a shaker. The solutions were slowly cooled down to room temperature with two cycles.

### Example 2-11. Solid Form 11 of Compound A

Solid Form 11 of Compound A was obtained from amorphous freebase of Compound A via sonication induced nucleation. About 30 mg of amorphous freebase of Compound A was dissolved in EtOAc to obtain a saturated solution under heating. The resulting solution was sonicated for one day.

### Example 2-12. Solid Form 12 of Compound A

Solid Form 12 of Compound A was obtained via slurring amorphous freebase of Compound A in THF/H₂O (a_{w}=0.6) at room temperature. About 30 mg of amorphous freebase of Compound A was suspended and stirred in THF/H₂O (a_{w}=0.6) in a 4 mL vial at room temperature.

Furthermore, although the foregoing has been described in some detail by way of illustrations and examples for purposes of clarity and understanding, it will be understood by those of skill in the art that numerous and various modifications can be made without departing from the spirit of the present disclosure. Therefore, it should be clearly understood that the forms disclosed herein are illustrative only and are not intended to limit the scope of the present disclosure, but rather to also cover all modification and alternatives coming with the true scope and spirit of the disclosure.

What is claimed:

1. Solid Form 1 of Compound A,
wherein said Solid Form 1 of Compound A has an X-ray powder diffraction pattern comprising one, two, three, four, or five peaks at about 4.91° 2θ±0.2° 2θ, about 5.98° 2θ±0.2° 2θ, about 9.77° 2θ±0.2° 2θ, about 10.64° 2θ±0.2° 2θ, or about 17.40° 2θ±0.2° 2θ.

2. The Solid Form of claim 1, wherein said Solid Form 1 has an X-ray powder diffraction pattern substantially similar to the X-ray powder diffraction pattern of FIG. 1A.

3. The Solid Form of claim 1, wherein said Solid Form 1 has a DSC thermogram substantially similar to the DSC thermogram of FIG. 1B.

4. The Solid Form of claim 1, wherein said Solid Form 1 has a TGA thermogram substantially similar to the TGA thermogram of FIG. 1B.

5. The Solid Form of any one of claims 1-4, wherein said Solid Form 1 has DSC endotherm peak at about 126° C., about 149° C., and/or about 168° C.

6. The Solid Form of claim 5, wherein the first DSC endotherm peak at about 126° C., the second DSC endotherm peak at about 149° C., and the third DSC endotherm peak at about 168° C.

7. The Solid Form of any one of claims 1-6, wherein said Solid Form 1 has a weight loss of about 2.78 wt. % when heated from about room temperature to about 150° C.

8. The Solid Form of any one of claims 1-7, wherein said Solid Form 1 is substantially free of any other Solid Form of Compound A.

9. Solid Form 2 of Compound A,
wherein said Solid Form 2 has an X-ray powder diffraction pattern comprising one, two, three, four, or five peaks at about 4.22° 2θ±0.2° 2θ, about 4.99° 2θ±0.2° 2θ, about 5.74° 2θ±0.2° 2θ, about 10.17° 2θ±0.2° 2θ, or about 16.04° 2θ±0.2° 2θ.

10. The Solid Form of claim 9, wherein said Solid Form 2 has an X-ray powder diffraction pattern substantially similar to the X-ray powder diffraction pattern of FIG. 2A.

11. The Solid Form of claim 9, wherein said Solid Form 2 has a DSC thermogram substantially similar to the DSC thermogram of FIG. 2B.

12. The Solid Form of claim 9, wherein said Solid Form 2 has a TGA thermogram substantially similar to the TGA thermogram of FIG. 2B.

13. The Solid Form of any one of claims 9-12, wherein said Solid Form 2 has DSC endotherm peak at about 134° C. and/or about 169° C., and/or has DSC exotherm peak at about 153° C.

14. The Solid Form of claim 13, wherein the first DSC endotherm peak at about 134° C., the second DSC endotherm peak at about 169° C., and the first DSC exotherm peak at about 153° C.

15. The Solid Form of any one of claims 9-14, wherein said Solid Form 2 has a weight loss of about 1.20 wt. % when heated from about room temperature to about 150° C. 16 The Solid Form of any one of claims 9-15, wherein said Solid Form 2 is substantially free of any other Solid Form of Compound A.

17. Solid Form 3 of Compound A,
wherein said Solid Form 3 has an X-ray powder diffraction pattern comprising one, two, three, four, or five peaks at about 3.42° 2θ±0.2° 2θ, about 4.51° 2θ±0.2° 2θ, about 5.87° 2θ±0.2° 2θ, about 10.10° 2θ±0.2° 2θ, or about 16.67° 2θ±0.2° 2θ.

18. The Solid Form of claim 17, wherein said Solid Form 3 has an X-ray powder diffraction pattern substantially similar to the X-ray powder diffraction pattern of FIG. 3A.

19. The Solid Form of claim 17, wherein said Solid Form 3 has a DSC thermogram substantially similar to the DSC thermogram of FIG. 3B.

20. The Solid Form of claim 17, wherein said Solid Form 3 has a TGA thermogram substantially similar to the TGA thermogram of FIG. 3B.

21. The Solid Form of any one of claims 17-20, wherein said Solid Form 3 has DSC endotherm peak at about 119° C., about 140° C. and/or about 167° C., and/or has DSC exotherm peak at about 156° C.

22. The Solid Form of claim 21, wherein the first DSC endotherm peak at about 119° C., the second DSC endotherm peak at about 140° C., the third DSC endotherm peak at about 167° C., and the first DSC exotherm peak at about 156° C.

23. The Solid Form of any one of claims 17-22, wherein said Solid Form 3 has a weight loss of about 2.26 wt. % when heated from about room temperature to about 150° C. 24 The Solid Form of any one of claims 17-23, wherein said Solid Form 3 is substantially free of any other Solid Form of Compound A.

25. Solid Form 4 of Compound A,
wherein said Solid Form 4 has an X-ray powder diffraction pattern comprising one, two, three, four, or five peaks at about 4.16° 2θ±0.2° 2θ, about 5.18° 2θ±0.2° 2θ, about 5.61° 2θ±0.2° 2θ, about 10.39° 2θ±0.2° 2θ, or about 16.65° 2θ±0.2° 2θ.

26. The Solid Form of claim 25, wherein said Solid Form 4 has an X-ray powder diffraction pattern substantially similar to the X-ray powder diffraction pattern of FIG. 4.

27. The Solid Form of claim 25 or 26, wherein said Solid Form 4 is substantially free of any other Solid Form of Compound A.

28. Solid Form 5 of Compound A,
wherein said Solid Form 5 has an X-ray powder diffraction pattern comprising one, two, three, four, or five peaks at about 3.40° 2θ±0.2° 2θ, about 4.48° 2θ±0.2° 2θ, about 5.18° 2θ±0.2° 2θ, about 5.86° 2θ±0.2° 2θ, or about 10.30° 2θ±0.2° 2θ.

29. The Solid Form of claim 28, wherein said Solid Form 5 has an X-ray powder diffraction pattern substantially similar to the X-ray powder diffraction pattern of FIG. 5A.

30. The Solid Form of claim 28, wherein said Solid Form 5 has a DSC thermogram substantially similar to the DSC thermogram of FIG. 5B.

31. The Solid Form of claim 28, wherein said Solid Form 5 has a TGA thermogram substantially similar to the TGA thermogram of FIG. 5B.

32. The Solid Form of any one of claims 28-31, wherein said Solid Form 5 has a weight loss of about 9.08 wt. % when heated from about room temperature to about 150° C.

33. The Solid Form of any one of claims 28-32, wherein said Solid Form 5 is substantially free of any other Solid Form of Compound A.

34. Solid Form 6 of Compound A,
wherein said Solid Form 6 has an X-ray powder diffraction pattern comprising one, two, three, four, or five peaks at about 5.25° 2θ±0.2° 2θ, about 11.26° 2θ±0.2° 2θ, about 12.70° 2θ±0.2° 2θ, about 15.34° 2θ±0.2° 2θ, or about 16.71° 2θ±0.2° 2θ.

35. The Solid Form of claim 34, wherein said Solid Form 6 has an X-ray powder diffraction pattern substantially similar to the X-ray powder diffraction pattern of FIG. 6A.

36. The Solid Form of claim 34, wherein said Solid Form 6 has a DSC thermogram substantially similar to the DSC thermogram of FIG. 6B.

37. The Solid Form of claim 34, wherein said Solid Form 6 has a TGA thermogram substantially similar to the TGA thermogram of FIG. 6B.

38. The Solid Form of any one of claims 34-37, wherein said Solid Form 6 has DSC endotherm peak at about 142° C.

39. The Solid Form of any one of claims 34-38, wherein said Solid Form 6 has a weight loss of about 0.75 wt. % when heated from about room temperature to about 100° C.

40. The Solid Form of any one of claims 34-39, wherein said Solid Form 6 is substantially free of any other Solid Form of Compound A.

41. Solid Form 7 of Compound 7,
wherein said Solid Form 7 has an X-ray powder diffraction pattern comprising one, two, three, four, or five peaks at about 5.16° 2θ±0.2° 2θ, about 9.34° 2θ±0.2° 2θ, about 16.40° 2θ±0.2° 2θ, about 16.52° 2θ±0.2° 2θ, or about 22.44° 2θ±0.2° 2θ.

42. The Solid Form of claim 41, wherein said Solid Form 7 has an X-ray powder diffraction pattern substantially similar to the X-ray powder diffraction pattern of FIG. 7A.

43. The Solid Form of claim 41, wherein said Solid Form 7 has a DSC thermogram substantially similar to the DSC thermogram of FIG. 7B.

44. The Solid Form of claim 41, wherein said Solid Form 7 has a TGA thermogram substantially similar to the TGA thermogram of FIG. 7B.

45. The Solid Form of any one of claims 41-44, wherein said Solid Form 7 has a DSC endotherm peak at about 80° C. and/or about 169° C., and/or has DSC exotherm peak at about 143° C.

46. The Solid Form of claim 45, wherein the first DSC endotherm peak at about 80° C., the second DSC endotherm peak at about 169° C., and the first DSC exotherm peak at about 143° C.

47. The Solid Form of any one of claims 41-46, wherein said Solid Form 7 has a weight loss of about 18.1 wt. % when heated from about room temperature to about 150° C.

48. The Solid Form of any one of claims 41-47, wherein said Solid Form 7 is substantially free of any other Solid Form of Compound A.

49. Solid Form 8 of Compound 8,
wherein said Solid Form 8 has an X-ray powder diffraction pattern comprising one, two, three, four, or five peaks at about 5.02° 2θ±0.2° 2θ, about 11.81° 2θ±0.2° 2θ, about 15.03° 2θ±0.2° 2θ, about 16.41° 2θ±0.2° 2θ, or about 20.09° 2θ±0.2° 2θ.

50. The Solid Form of claim 49, wherein said Solid Form 8 has an X-ray powder diffraction pattern substantially similar to the X-ray powder diffraction pattern of FIG. 8A.

51. The Solid Form of claim 49, wherein said Solid Form 8 has a DSC thermogram substantially similar to the DSC thermogram of FIG. 8B.

52. The Solid Form of claim 49, wherein said Solid Form 8 has a TGA thermogram substantially similar to the TGA thermogram of FIG. 8B.

53. The Solid Form of any one of claims 49-52, wherein said Solid Form 8 has DSC endotherm peak at about 75° C. and/or about 99° C.

54. The Solid Form of claim 53, having a first DSC endotherm peak at about 75° C., and a second DSC endotherm peak at about 99° C.

55. The Solid Form of any one of claims 49-54, wherein said Solid Form 8 has a weight loss of about 6.0 wt. % when heated from about room temperature to about 125° C., and/or about 9.55 wt. % when heated from about 125° C. to about 252° C.

56. The Solid Form of any one of claims 49-55, wherein said Solid Form 8 is substantially free of any other Solid Form of Compound A.

57. Solid Form 9 of Compound A,
wherein said Solid Form 9 has an X-ray powder diffraction pattern one, two, three, four, or five peaks at about 4.20° 2θ±0.2° 2θ, about 4.47° 2θ±0.2° 2θ, about 5.20° 2θ±0.2° 2θ, about 6.43° 2θ±0.2° 2θ, or about 10.46° 2θ±0.2° 2θ.

58. The Solid Form of claim 57, wherein said Solid Form 9 has an X-ray powder diffraction pattern substantially similar to the X-ray powder diffraction pattern of FIG. 9A.

59. The Solid Form of claim 57, wherein said Solid Form 9 has a DSC thermogram substantially similar to the DSC thermogram of FIG. 9B.

60. The Solid Form of claim 57, wherein said Solid Form 9 has a TGA thermogram substantially similar to the TGA thermogram of FIG. 9B.

61. The Solid Form of any one of claims 57-60, wherein said Solid Form 9 has DSC endotherm peak at about 130° C., and/or about 168° C., and/or has DSC exotherm peak at about 143° C.

62. The Solid Form of claim 61, having a first DSC endotherm peak at about 130° C., a second DSC endotherm peak at about 168° C., and a first DSC exotherm peak at about 143° C.

63. The Solid Form of any one of claims 57-62, wherein said Solid Form 9 has a weight loss of about 0.99 wt. % when heated from about room temperature to about 150° C.

64. The Solid Form of any one of claims 57-63, wherein said Solid Form 9 is substantially free of any other Solid Form of Compound A.

65. Solid Form 10 of Compound A,
wherein said Solid Form 10 has an X-ray powder diffraction pattern comprising one, two, three, four, or five peaks at about 4.96° 2θ±0.2° 2θ, about 5.13° 2θ±0.2° 2θ, about 5.53° 2θ±0.2° 2θ, about 10.22° 2θ±0.2° 2θ, or about 11.15° 2θ±0.2° 2θ.

66. The Solid Form of claim 65, wherein said Solid Form 10 has an X-ray powder diffraction pattern substantially similar to the X-ray powder diffraction pattern of FIG. 10.

67. The Solid Form of claim 65 or 66, wherein said Solid Form 10 is substantially free of any other Solid Form of Compound A.

68. Solid Form 11 of Compound A,
wherein said Solid Form 11 has an X-ray powder diffraction pattern comprising one, two, three, four, or five peaks at about 4.38° 2θ±0.2° 2θ, about 5.20° 2θ±0.2° 2θ, about 6.43° 2θ±0.2° 2θ, about 10.45° 2θ±0.2° 2θ, or about 12.61° 2θ±0.2° 2θ.

69. The Solid Form of claim 68, wherein said Solid Form 11 has an X-ray powder diffraction pattern substantially similar to the X-ray powder diffraction pattern of FIG. 11A.

70. The Solid Form of claim 68, wherein said Solid Form 11 has a DSC thermogram substantially similar to the DSC thermogram of FIG. 11B.

71. The Solid Form of claim 68, wherein said Solid Form 11 has a TGA thermogram substantially similar to the TGA thermogram of FIG. 11B.

72. The Solid Form of any one of claims 68-71, wherein said Solid Form 11 has DSC endotherm peak at about 134° C., and/or about 168° C.

73. The Solid Form of claim 72, having a first DSC endotherm peak at about 134° C., and a second DSC endotherm peak at about 168° C.

74. The Solid Form of any one of claims 68-73, wherein said Solid Form 11 has a weight loss of about 1.13 wt. % when heated from about room temperature to about 100° C.

75. The Solid Form of any one of claims 68-74, wherein said Solid Form 11 is substantially free of any other Solid Form of Compound A.

76. Solid Form 12 of Compound A,
wherein said Solid Form 12 has an X-ray powder diffraction pattern comprising one, two, three, four, or five peaks at about 5.23° 2θ±0.2° 2θ, about 15.64° 2θ±0.2° 2θ, about 16.48° 2θ±0.2° 2θ, about 16.67° 2θ±0.2° 2θ, or about 21.34° 2θ±0.2° 2θ.

77. The Solid Form of claim 76, wherein said Solid Form 12 has an X-ray powder diffraction pattern substantially similar to the X-ray powder diffraction pattern of FIG. 12A.

78. The Solid Form of claim 76, wherein said Solid Form 12 has a DSC thermogram substantially similar to the DSC thermogram of FIG. 12B.

79. The Solid Form of claim 76, wherein said Solid Form 12 has a TGA thermogram substantially similar to the TGA thermogram of FIG. 12B.

80. The Solid Form of any one of claims 76-79, wherein said Solid Form 12 has DSC endotherm peak at about 90° C.

81. The Solid Form of any one of claims 76-80, wherein said Solid Form 12 has a weight loss of about 8.66 wt. % when heated from about room temperature to about 150° C.

82. The Solid Form of any one of claims 76-81, wherein said Solid Form 12 is substantially free of any other Solid Form of Compound A.

83. A pharmaceutical composition comprising an effective amount of the Solid Form of any one of claims 1-82, and a pharmaceutically acceptable carrier, diluent, excipient or combination thereof.

84. A method for ameliorating or treating a malignant growth or tumor in a subject in need thereof, said method comprising administering to the subject an effective amount of the Solid Form of any one of claims 1-82, or the pharmaceutical composition of claim 83.

85. A method for ameliorating or treating a malignant growth or tumor in a subject in need thereof, said method comprising contacting the malignant grown or tumor with an effective amount of the Solid Form of any one of claims 1-82, or the pharmaceutical composition of claim 83.

86. The method of claim 84 or 85, wherein the malignant growth or tumor is due to a cancer, wherein said cancer is selected from a breast cancer, a cervical cancer, an ovarian cancer, a uterine cancer, a vaginal cancer, a vulvar cancer, a brain cancer, a cervicocerebral cancer, an esophageal cancer, a thyroid cancer, a small cell cancer, a non-small cell cancer, a lung cancer, a stomach cancer, a gallbladder/bile duct cancer, a liver cancer, a pancreatic cancer, a colon cancer, a rectal cancer, a choriocarcinoma, an uterus body cancer, an uterocervical cancer, a renal pelvis/ureter cancer, a bladder cancer, a prostate cancer, a penis cancer, a testicular cancer, a fetal cancer, a Wilms' cancer, a skin cancer, a malignant melanoma, a neuroblastoma, an osteosarcoma, an Ewing's tumor, a soft part sarcoma, an acute leukemia, a chronic lymphatic leukemia, a chronic myelocytic leukemia, polycythemia vera, a malignant lymphoma, multiple myeloma, a Hodgkin's lymphoma and a non-Hodgkin's lymphoma.

87. The method of claim 86, wherein the cancer is a breast cancer, a cervical cancer, an ovarian cancer, a uterine cancer, a vaginal cancer, or a vulvar cancer.

88. Use of an effective amount of a compound that is the Solid Form of any one of claims 1-82, or the pharmaceutical composition of claim 83, in the manufacture of a medicament for ameliorating or treating a malignant growth or tumor, wherein the malignant growth or tumor is due to a cancer selected from a breast cancer, a cervical cancer, an ovarian cancer, a uterine cancer, a vaginal cancer, a vulvar cancer, a brain cancer, a cervicocerebral cancer, an esophageal cancer, a thyroid cancer, a small cell cancer, a non-small cell cancer, a lung cancer, a stomach cancer, a gallbladder/bile duct cancer, a liver cancer, a pancreatic cancer, a colon cancer, a rectal cancer, a choriocarcinoma, an uterus body cancer, an uterocervical cancer, a renal pelvis/ureter cancer, a bladder cancer, a prostate cancer, a penis cancer, a testicular cancer, a fetal cancer, a Wilms' cancer, a skin cancer, a malignant melanoma, a neuroblastoma, an osteosarcoma, an Ewing's tumor, a soft part sarcoma, an acute leukemia, a chronic lymphatic leukemia, a chronic myelocytic leukemia, polycythemia vera, a malignant lymphoma, multiple myeloma, a Hodgkin's lymphoma and a non-Hodgkin's lymphoma.

89. The use of claim 88, wherein the cancer is a breast cancer, a cervical cancer, an ovarian cancer, a uterine cancer, a vaginal cancer or a vulvar cancer.

90. A compound that is the Solid Form of any one of claims 1-82, or the pharmaceutical composition of claim 83, for use in ameliorating or treating a malignant growth or tumor, wherein the malignant growth or tumor is due to a cancer selected from a breast cancer, a cervical cancer, an ovarian cancer, a uterine cancer, a vaginal cancer, a vulvar cancer, a brain cancer, a cervicocerebral cancer, an esophageal cancer, a thyroid cancer, a small cell cancer, a non-small cell cancer, a lung cancer, a stomach cancer, a gallbladder/bile duct cancer, a liver cancer, a pancreatic cancer, a colon cancer, a rectal cancer, a choriocarcinoma, an uterus body cancer, an uterocervical cancer, a renal pelvis/ureter cancer, a bladder cancer, a prostate cancer, a penis cancer, a testicular cancer, a fetal cancer, a Wilms' cancer, a skin cancer, a malignant melanoma, a neuroblastoma, an osteosarcoma, an Ewing's tumor, a soft part sarcoma, an acute leukemia, a chronic lymphatic leukemia, a chronic myelocytic leukemia, polycythemia vera, a malignant lymphoma, multiple myeloma, a Hodgkin's lymphoma and a non-Hodgkin's lymphoma.

91. The compound of claim 90, wherein the cancer is a breast cancer, a cervical cancer, an ovarian cancer, a uterine cancer, a vaginal cancer, or a vulvar cancer.

92. A method for inhibiting the activity of WEE1 in a subject in need thereof, said method comprising administering to the subject an effective amount of the Solid Form of any one of claims 1-82, or the pharmaceutical composition of claim 83.

93. A method of preparing any of Solid Forms 1-12 of Compound A, wherein said method is substantially as described in Table 2 and/or any one of Examples 2-1 to 2-12.

94. A method of preparing Solid Form 1 of Compound A, said method comprising using a slurry of amorphous Compound A, wherein said slurry comprises the solvents of n-heptane and n-BuOH, and optionally wherein said slurry is heated to about 50° C.

95. The method of claim 94, wherein the ratio of n-heptane and n-BuOH is about 8:2.

96. The method of any one of claims 93-95, wherein Solid Form 1 of Compound A is according to any one of claims 1-8.

97. A method of preparing Solid Form 2 of Compound A, said method comprising anti-solvent addition, reverse anti-solvent addition, solid vapor diffusion, liquid vapor diffusion, a slurry at room temperature, a slurry at 50° C., slow evaporation, fast evaporation, slow cooling, and/or temperature cycling.

98. The method of claim 97, wherein said anti-solvent addition comprises the use of any one of the following solvent systems:

| Combination | Solvent 1 | Solvent 2 |
|---|---|---|
| 1 | Acetone | MTBE |
| 2 | MeCN | MTBE |
| 3 | Pyridine | MTBE |
| 4 | DCM | n-heptane |
| 5 | DCM | MTBE |
| 6 | Dioxane | Toluene |
| 7 | n-PrOH | MTBE |
| 8 | EtOAc | MTBE |
| 9 | IPAc | MTBE |
| 10 | 2-Butanone | MTBE |

99. The method of claim 97, wherein said reverse anti-solvent addition comprises the use of any one of the following solvent systems:

| Combination | Solvent 1 | Solvent 2 |
|---|---|---|
| 1 | DMF | MTBE |
| 2 | Diisopropyl ether | n-heptane |
| 3 | Diisopropyl ether | MTBE |
| 4 | Xylene | MTBE |

100. The method of claim 97, wherein said solvent vapor diffusion comprises the use of diisopropyl ether, methyl isobutyl ketone (MIBK), MeOH, and/or isopropyl acetate (IPAc).

101. The method of claim 97, wherein said liquid vapor diffusion comprises the use of any one of the following solvent systems:

| Combination | Solvent 1 | Solvent 2 |
|---|---|---|
| 1 | DMF | MTBE |
| 2 | n-BuOH | MTBE |
| 3 | EtOH | MTBE |
| 4 | EtOH | Toluene |
| 5 | CHCl₃ | IPAc |
| 6 | CHCl₃ | Toluene |

102. The method of claim 97, wherein said slurry comprises a solvent that is EtOAc and is optionally at room temperature.

103. The method of claim 97, wherein said slurry comprises a solvent system that is MIBK, n-heptane/xylene, n-heptane/2-MeTHF, or n-heptane/MIBK, and optionally wherein said slurry is at 50° C. and/or the solvents are in a ratio of about 8:2.

104. The method of claim 97, wherein said fast evaporation comprises the use of a solvent that is toluene.

105. The method of claim 97, wherein said slow cooling comprises the use of a solvent that is diisopropylether.

106. The method of claim 97, wherein said temperature cycling comprises the use of a solvent that is EtOAc or a combination of n-heptane/xylene, and optionally wherein the combination of n-heptane/xylene is present in a ratio of about 2:1 (n-heptane:xylene).

107. The method of any one of claims 97-106, wherein the Solid Form 2 of Compound A is according to any one of claims 9-16.

108. A method of preparing Solid Form 3 of Compound A, said method comprising using an anti-solvent addition, liquid vapor diffusion, and/or a slurry at about 50° C.

109. The method of claim 108, wherein said anti-solvent addition comprises the use of a solvent combination selected from: n-BuOH/MTBE, dioxane/MTBE, and IPA/MTBE.

110. The method of claim 108, wherein said liquid vapor diffusion comprises the use of a solvent system comprising CHCl₃ and cyclohexane.

111. The method of claim 108, wherein said slurry comprises a solvent that is cyclohexane.

112. The method of any one of claims 108-111, wherein said Solid Form 3 is according to any one of claims 17-24.

113. A method of preparing Solid Form 4 of Compound A, said method comprising using a slurry at about room temperature.

114. The method of claim 113, wherein said slurry comprises a solvent system of n-heptane and CH₂Cl₂, and optionally wherein said solvents are in a ratio of about 8:2 (n-heptane:CH₂Cl₂).

115. The method of claim 113 or 114, wherein Solid Form 4 of Compound A is according to any one of claims 25-27.

116. A method of preparing Solid Form 5 of Compound A, said method comprising using anti-solvent addition, reverse anti-solvent addition, and/or a slurry at about room temperature.

117. The method of claim 116, wherein said anti-solvent addition comprises the use of a solvent system selected from: benzene/MTBE, MeOH/MTBE, and THF/MTBE.

118. The method of claim 116, wherein said reverse anti-solvent addition comprises the use of a solvent system that is pyridine/toluene.

119. The method of claim 116, wherein said slurry comprises a solvent system of n-heptane and dioxane, and optionally wherein said solvents are in a ratio of about 8:2 (n-heptane:dioxane).

120. The method of any one of claims 116-119, wherein said Solid Form 5 is according to any one of claims 28-33.

121. A method of preparing Solid Form 6 of Compound A, said method comprising using reverse anti-solvent addition or liquid vapor diffusion.

122. The method of claim 121, wherein said reverse anti-solvent addition comprises the use of a solvent system that is acetic acid/MTBE or acetic acid/toluene.

123. The method of claim 121, wherein said liquid vapor diffusion comprises the use of a solvent system that is acetic acid/MTBE.

124. The method of any one of claims 121-123, wherein Solid Form 6 is according to any one of claims 34-40.

125. A method of preparing Solid Form 7 of Compound A, said method comprising using reverse anti-solvent addition, liquid vapor diffusion, and/or polymer induced crystallization.

126. The method of claim 125, wherein said reverse anti-solvent addition comprises the use of a solvent system selected from benzene/toluene, n-BuOH/n-heptane, and n-PrOH/n-heptane.

127. The method of claim 125, wherein said liquid vapor diffusion comprises the use of a solvent system that is n-BuOH/n-heptane.

128. The method of claim 125, wherein said polymer induced crystallization comprises the use of a polymer that is hydroxypropyl methylcellulose (HPMC) and/or a solvent system that is MTBE/IPA, and optionally said solvents are in a ratio of about 1:1 (MTBE:IPA).

129. The method of any one of claims 125-128, wherein said Solid Form 7 is according to any one of claims 41-48.

130. A method of preparing Solid Form 8 of Compound A, said method comprising using anti-solvent addition and/or a slurry at room temperature.

131. The method of claim 130, wherein said anti-solvent addition comprises the use of a solvent system that is DMAc/n-heptane.

132. The method of claim 130, wherein said slurry comprises a solvent system that is MTBE/DMF, and optionally in a ratio of about 6:4 (v/v) (MTBE:DMF).

133. The method of any one of claims 130-132, wherein said Solid Form 8 is according to any one of claims 49-56.

134. A method of preparing Solid Form 9 of Compound A, said method comprising using reverse anti-solvent addition.

135. The method of claim 134, wherein said reverse anti-solvent addition comprises a solvent system comprising xylene and n-heptane.

136. The method of claim 134 or 135, wherein said Solid Form 9 is according to any one of claims 57-64.

137. A method of preparing Solid Form 10 of Compound A, said method comprising using slow cooling, and optionally comprising the use of a solvent that is CHCl₃.

138. The method of claim 137, wherein said Solid Form 10 is according to any one of claims 65-67.

139. A method of preparing Solid Form 11 of Compound A, said method comprising using sonication induced nucleation, and optionally comprising the use of a solvent that is EtOAc.

140. The method of claim 139, wherein said Solid Form 11 is according to any one of claims 68-75.

141. A method of preparing Solid Form 12 of Compound A, said method comprising using a slurry at room temperature.

142. The method of claim 141, wherein said slurry comprises a solvent system that is THF/H₂O, and optionally having a water activity (a_{w}) of about 0.2, about 0.4, about 0.6, or about 0.8.

143. The method of claim 141 of 142, wherein said Solid Form 12 is according to any one of claims 76-82.
